(19) United States
(12) Patent Application Publication
(10) Pub. No.: US 2011/0112079 A1
(43) Pub. Date: May 12, 2011

(54) PHOSPHODIESTERASE INHIBITORS

(76) Inventors: Craig J. Thomas, Gaithersburg, MD (US); Menghang Xia, Potomac, MD (US); Amanda P. Skoumbourdis, Langhorne, PA (US); Christopher A. LeClair, Gaithersburg, MD (US); Ruili Huang, Rockville, MD (US); Martin J. Walsh, Olney, MD (US)

(21) Appl. No.: 12/812,291

(22) PCT Filed: Jan. 8, 2009
(86) PCT No.: PCT/US09/00105

§ 371 Date: Sep. 29, 2010

Related U.S. Application Data
(60) Provisional application No. 61/020,079, filed on Jan. 9, 2008.
(60) Provisional application No. 61/080,969, filed on Jul. 15, 2008.
(60) Provisional application No. 61/084,934, filed on Jul. 30, 2008.

(51) Int. Cl. A61K 31/542; C07D 487/04; A61K 31/5025; C12N 5/00; A61P 29/00; A61P 11/06; A61P 17/06; A61P 19/02; A61P 37/06; A61P 3/10; A61P 25/28; A61P 25/16; A61P 25/24; C07D 513/04

(52) U.S. Cl. 514/222.8; 544/236; 514/248; 435/375; 544/10

(57) ABSTRACT

The invention related to compounds for formula I useful for inhibiting phosphodiesterase-4.

This application claims priority under 35 U.S.C. 119(e) to U.S. Provisional Application Ser. No. 61/020,079 filed Jan. 9, 2008, U.S. Provisional Application Ser. No. 61/080,969 filed Jul. 15, 2008, and U.S. Provisional Application Ser. No. 61/084,934 filed Jul. 30, 2008, the contents of which applications are specifically incorporated herein in their entireties.

### GOVERNMENT FUNDING

The invention described herein was developed with support from the National Institutes of Health. The U.S. Government has certain rights in the invention.

### FIELD OF THE INVENTION

The invention is related to compounds useful for inhibiting phosphodiesterases.

### BACKGROUND OF THE INVENTION

Inflammation of the airways is central to the airway dysfunction that characterizes pulmonary diseases such as asthma. Typically, the airway wall is infiltrated by a variety of cells including mast cells, eosinophils and T lymphocytes, which have deviated towards a T(H)₂ phenotype. Together, these cells release a plethora of factors including interleukin (IL)-4, IL-5, granulocyte/macrophage colony-stimulating factor and eotaxin that ultimately cause the histopathology and symptoms of asthma. Glucocorticosteroids are currently the only drugs that effectively impact this inflammation and resolve, to a greater or lesser extent, compromised lung function. However, steroids are nonselective and generally unsuitable for pediatric use. New drugs are clearly required.

One group of therapeutic agents for asthma are inhibitors of cyclic AMP-specific phosphodiesterase (PDE). For example, theophylline is a prototypic PDE inhibitor. PDE is a generic term that refers to at least 11 distinct enzyme families that hydrolyze cAMP and/or cGMP. Phosphodiesterase-4 (PDE4) inhibitors are useful as anti-inflammatory drugs especially in airway diseases. They suppress the release of inflammatory signals, (e.g., cytokines), and inhibit the production of reactive oxygen species. PDE4 inhibitors have utility as non-steroidal disease controllers in inflammatory airway diseases such as asthma, chronic obstructive pulmonary disease (COPD) and rhinitis. PDE4 inhibitors may also act as anti-depression agents and have also recently been proposed for use in antipsychotic medications.

### SUMMARY OF THE INVENTION

The invention is directed to compounds useful for inhibiting phosphodiesterases, for example, phosphodieasterase-4 (PDE-4). PDE-4 inhibitors are useful for the treatment of inflammation, for example, asthma and chronic obstructive pulmonary disorders (COPD, emphysema & bronchitis), as well as for treatment of depression, psychosis and memory problems.

One aspect of the invention is a compound of formula I:

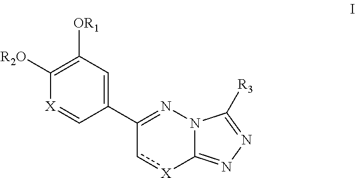

wherein:
   X is CH, CH₂, or heteroatom;
   each R₁ and R₂ is separately alkyl, haloalkyl, cycloalkyl, heterocycloalkyl, or aryl, where the alkyl, cycloalkyl, heterocycloalkyl, or aryl can be covalently linked to the oxygen via a lower alkyl; and
   R₃ is aryl substituted with 1-3 alkyl, alkenyl, alkynyl, cycloalkyl, cycloalkenyl, aryl, heteroaryl, OH, O-alkyl, SH, S-alkyl, NH₂, NH-alkyl, N-dialkyl, NH-acyl, NH-aryl, OCO-alkyl, SCO-alkyl, SOH, SO-alkyl, SO₂H, SO₂-alkyl, SO₂NH₂, SO₂NH-alkyl, SO₂N-dialkyl, CF₃, F, Cl, Br, or I groups.

In some embodiments, the X heteroatom is O, S, N or NH. For example, the compound can have one of the following formulae:

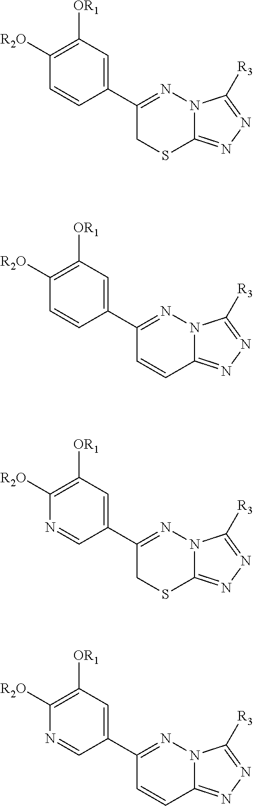

wherein:
   each R₁ and R₂ is separately alkyl, haloalkyl, cycloalkyl, cycloalkylhalo, heterocycloalkyl, or aryl, where the alkyl, cycloalkyl, cycloalkylhalo, heterocycloalkyl, or aryl can be covalently linked to the oxygen via a lower alkyl;
   R₃ is aryl substituted with 1-3 alkyl, alkenyl, alkynyl, cycloalkyl, cycloalkenyl, aryl, heteroaryl, OH, O-alkyl, SH, S-alkyl, —NH₂, NH-alkyl, N-dialkyl, NH-acyl, NH-aryl, OCO-alkyl, SCO-alkyl, SOH, SO-alkyl, SO₂H, SO₂-alkyl, SO₂NH₂, SO₂NH-alkyl, SO₂N-dialkyl, CF₃, F, Cl, Br, or I groups.

In other embodiments, the X can be N or CH in the following ring:

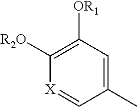

In other embodiments, the X can be S or CH in the following ring:

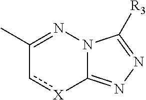

The R₃ moiety in the compounds of the invention can be an aryl, for example, a phenyl or naphthyl group. In some embodiments, the R₃ aryl group is a phenyl group. The R₃ aryl group is often substituted with 1-3 lower alkyl, lower alkoxy or lower alkylhalide groups. Halide atoms such as Br, Cl, F and I atoms can be present on the R₃ aryl group. For example, the R₁ and R₂ haloalkyl groups or cycloalkylhalo groups can be lower alkyl or lower cycloalkyl groups that are substituted with 1-3 halide atoms. In some compounds of the invention, the R₁ and R₂ alkyl groups are lower alkyl groups, for example, R₁ and R₂ can each be methyl or ethyl.

When R₃ is phenyl, for example, the phenyl can have 1-3 alkyl, alkenyl, alkynyl, cycloalkyl, cycloalkenyl, aryl, heteroaryl, OH, O-alkyl, SH, S-alkyl, NH₂, NH-alkyl, N-dialkyl, NH-acyl, NH-aryl, OCO-alkyl, SCO-alkyl, SOH, SO-alkyl, SO₂H, SO₂-alkyl, SO₂NH₂, SO₂NH-alkyl, SO₂N-dialkyl, CF₃, F, Cl, Br, or I groups. However, in some embodiments, the R₃ phenyl group is substituted with 2 such groups. One example of an R₃ group that gives rise to highly potent phosphodiesterase-4 inhibitors is dimethoxyphenyl. Thus, for example, the compounds of the invention can have an R₃ group with the following structure:

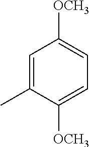

Examples of compounds of the invention include those having any of the following formulae:

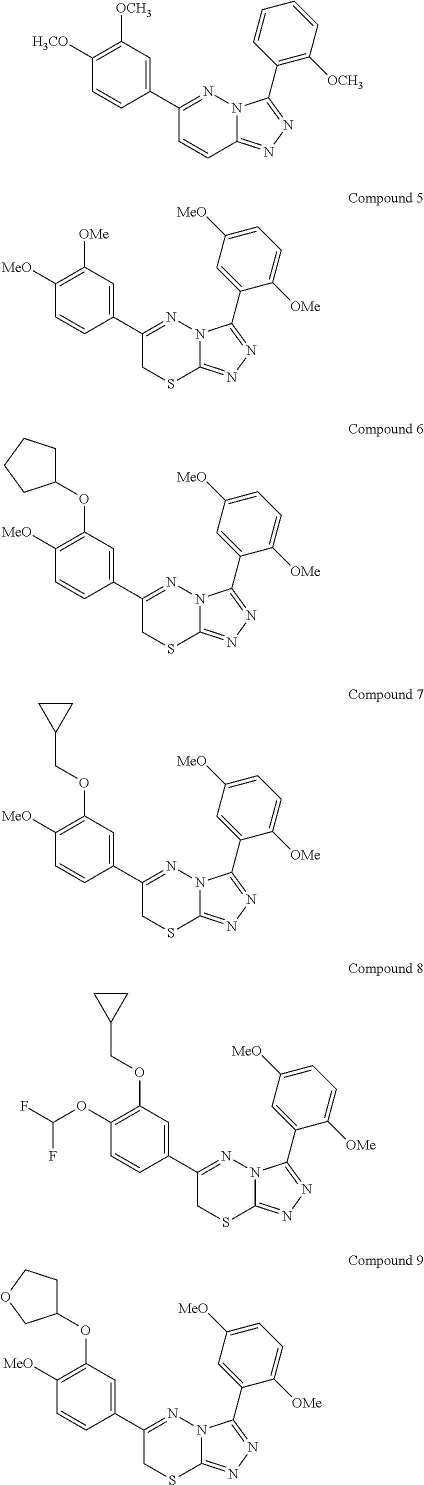

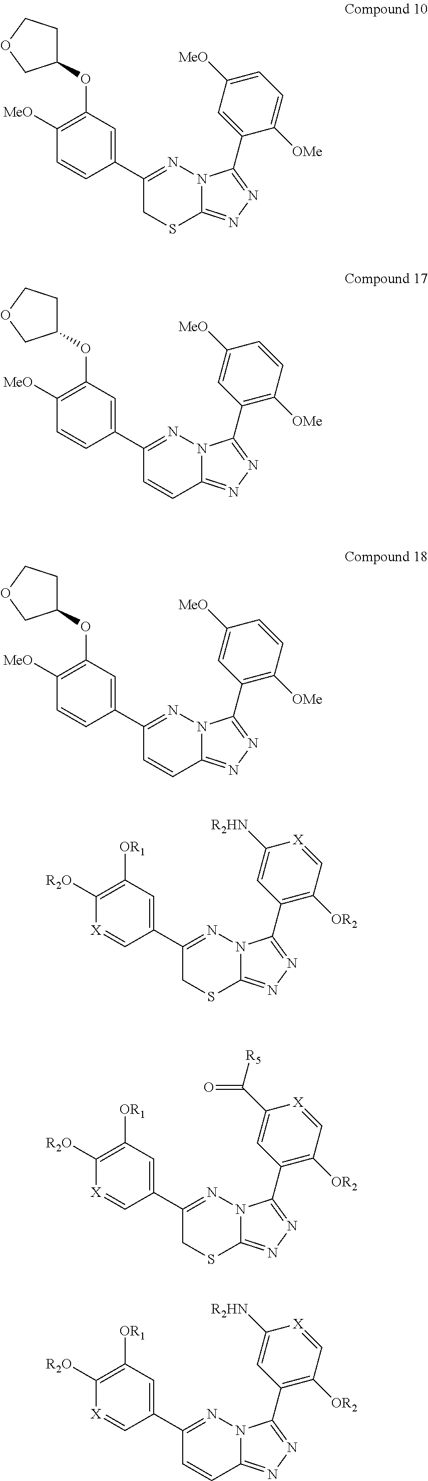

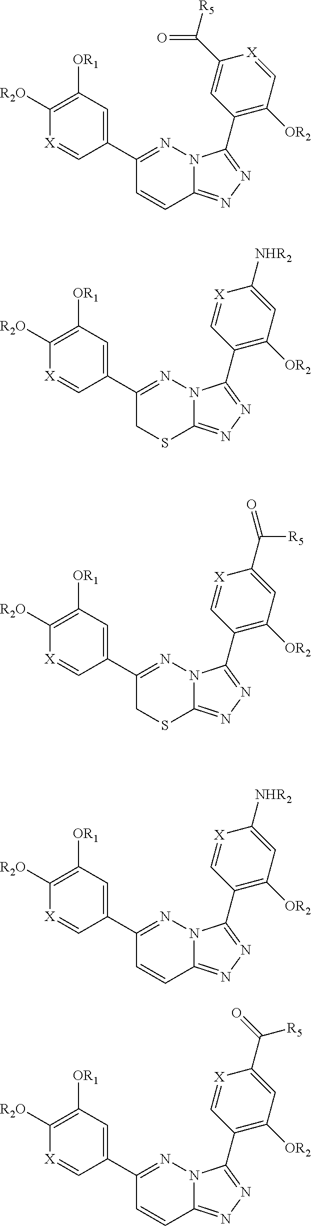

wherein:
   X is CH or heteroatom;
   each R₁ and R₂ is separately alkyl, haloalkyl, cycloalkyl, cycloalkylhalo, heterocycloalkyl, or aryl, where the alkyl, cycloalkyl, cycloalkylhalo, heterocycloalkyl, or aryl can be covalently linked to the oxygen via a lower alkyl; and
   R₅ is amide, ester, alkyl or aryl.

Another aspect of the invention is a composition that includes a carrier and an effective amount of at least one compound of the invention. The carrier employed can be a pharmaceutically acceptable carrier. The effective amount of the compound can be a therapeutically effective amount. One example of a therapeutically effective amount of the present compounds for administration to a mammal is about 0.0001 mg/kg to about 500 mg/kg.

Another aspect of the invention is a method for inhibiting phosphodiesterase-4 in a mammalian cell, comprising administering to the mammal an effective amount of the composition of any of claims 12-14 to thereby inhibit phosphodiesterase-4 in the mammal. Such an effective amount can, for example, be effective for inhibiting at least 30% or at least 50%, or at least 60%, or at least 70% of the phosphodiesterase-4. One example of an effective amount of the present compounds for administration to a mammal is about 0.0001 mg/kg to about 500 mg/kg.

In some embodiments, the mammalian cell in a mammal. For example, the phosphodiesterase-4 can be inhibited within a cell in a mammal to treat any one of the following diseases or disorders: inflammation, acute airway disorders, chronic airway disorders, inflammatory airway disorders, allergen-induced airway disorders, bronchitis, allergic bronchitis, bronchial asthma, emphysema, chronic obstructive pulmonary disease, dermatoses, proliferative dermatoses, inflammatory dermatoses, allergic dermatosis, psoriasis (vulgaris), toxic eczema, allergic contact eczema, atopic eczema, seborrhoeic eczema, Lichen simplex, sunburn, pruritus in the anogenital area, alopecia areata, hypertrophic scars, discoid lupus erythematosus, follicular and widespread pyodermias, endogenous and exogenous acne, acne rosacea, proliferative, inflammatory and allergic skin disorders, rheumatoid arthritis, rheumatoid spondylitis, osteoarthritis, arthritis, AIDS, multiple sclerosis, graft versus host reaction, allograft rejection, shock, septic shock, endotoxin shock, gram-negative sepsis, toxic shock syndrome, adult respiratory distress syndrome, Crohn's disease, ulcerative colitis, inflammatory bowel disease, allergies, allergic rhinitis, sinusitis, chronic rhinitis, chronic sinusitis, allergic conjunctivitis, nasal polyps, cardiac insufficiency, erectile dysfunction, kidney colic, ureter colic in connection with kidney stones, diabetes, diabetes insipidus, cerebral senility, senile dementia (Alzheimer's disease), memory impairment associated with Parkinson's disease or multiinfarct dementia, depression, psychosis, arteriosclerotic dementia or a combination thereof.

The compounds of the invention can be used for the preparation of medicament, for example, to treat any of the diseases, disorders and conditions recited herein.

Another aspect of the invention is a method for inhibiting phosphodiesterase-4 in a mammal, comprising administering to the mammal an effective amount of a compound of the invention or a combination thereof, to thereby inhibit phosphodiesterase-4 in the mammal.

In some embodiments, the phosphodiesterase-4 is inhibited in a mammal to treat any one of the following diseases or disorders: inflammation, acute airway disorders, chronic airway disorders, inflammatory airway disorders, allergen-induced airway disorders, bronchitis, allergic bronchitis, bronchial asthma, emphysema, chronic obstructive pulmonary disease, dermatoses, proliferative dermatoses, inflammatory dermatoses, allergic dermatosis, psoriasis (vulgaris), toxic eczema, allergic contact eczema, atopic eczema, seborrhoeic eczema, Lichen simplex, sunburn, pruritus in the anogenital area, alopecia areata, hypertrophic scars, discoid lupus erythematosus, follicular and widespread pyodermias, endogenous and exogenous acne, acne rosacea, proliferative, inflammatory and allergic skin disorders, rheumatoid arthritis, rheumatoid spondylitis, osteoarthritis, arthritis, AIDS, multiple sclerosis, graft versus host reaction, allograft rejection, shock, septic shock, endotoxin shock, gram-negative sepsis, toxic shock syndrome, adult respiratory distress syndrome, Crohn's disease, ulcerative colitis, inflammatory bowel disease, allergies, allergic rhinitis, sinusitis, chronic rhinitis, chronic sinusitis, allergic conjunctivitis, nasal polyps, cardiac insufficiency, erectile dysfunction, kidney colic, ureter colic in connection with kidney stones, diabetes, diabetes insipidus, cerebral senility, senile dementia (Alzheimer's disease), memory impairment associated with Parkinson's disease or multiinfarct dementia, depression, psychosis, arteriosclerotic dementia or a combination thereof.

### DESCRIPTION OF THE FIGURES

FIG. 1 schematically illustrates cyclic nucleotide regulation of several physiological pathways and its effects thereon. Thus, cGMP is formed via guanylate cyclase (GC) or via nitrous oxide (NO) stimulated guanylate cyclase activation. cAMP is similarly formed by adenylate cyclase, which is activated via G proteins (Gs), which interact with G-protein coupled receptors (GPCRs). cGMP and cAMP regulate several effectors including PICA (protein kinase A), PKG (protein kinase G), GEF (guanine-nucleotide exchange factor) and CNG channels (cyclic-nucleotide gated ion channels). Numerous phosphodiesterases convert cAMP and cGMP to 5′-AMP and 5′-GMP, respectively. Inhibition of such phosphodiesterases therefore prolongs the half-lives of cGMP and cAMP.

FIG. 2 illustrates some procedures that can be used to synthesize the substituted 7H-[1,2,4]triazolo[3,4-b][1,3,4]thiadiazines compounds of the invention. Reagents and conditions used for steps (i) through (vi): (i) cat. H₂SO₄ in methanol (MeOH) incubated at room temperature for 12 hours; (ii) hydrazine in ethanol (EtOH) refluxed for 12 hours; (iii) KOH, EtOH; then CS₂ at room temperature for 12 h; (iv) hydrazine monohydrate, H₂O, refluxed for 3 hours, then concentrated HCl was added; (v) Br₂ in CHCl₃, incubated at room temperature for 5 min., then the mixture was refluxed 30 minutes to 4 hours; and (vi) EtOH added and the mixture was incubated at 105° C. for 4 hours.

FIGS. 3A-D demonstrate that the phosphodiesterase inhibitors of the invention are active intracellularly. Inhibition by compounds 1 (FIG. 3A), 5 (FIG. 3B), 10 (FIG. 3C) and 18 (FIG. 3D) was observed in a cell-based cyclic nucleotide-gated cation channel biosensor assay. The concentrations at which the compounds exhibited 50% activity (i.e., the EC₅₀ values) for compounds 1, 5, 10 and 18 are as follows: EC₅₀ for 1=131.5 nM; EC₅₀ for 5=18.7 nM; EC₅₀ for 10=2.3 nM; ECₛₒ for 18=34.2 nM. The data shown are from four separate experiments.

FIGS. 4A-C further illustrates inhibition of phosphodiesterase-4 intracellularly by compounds of the invention using a protein fragmentation and complementation assay similar to that described in Stefan et al., Proc. Natl. Acad. Sci. USA. 104: 16916-16921 (2007). The luminescence signal is a measure of β₂AR signaling to PKA, which is reduced when phosphodiesterase-4 is inhibited. Stable β₂AR-HEK293 cells were transiently transfected with the PKA reporter Reg-F[1]:Cat-F[2]. FIG. 4A shows how various pretreatments affect the luminescence signal, including the selective β₂AR-antagonist 20 (1 μM), the known PDE inhibitor 1 (100 μM; 30 min) and/or compound 19 (1 μM, 30 min) (mean±s.d. from independent triplicates). The isoproterenol (19) was able to reduce luminescence, indicating dissociation of the Rluc biosensor complex and consequent activation of PKA catalytic activity. Pretreatment with the selective β₂AR inverse agonist IC118551 (20), which can decrease basal β₂AR activity, was able to prevent the effects of 19. FIG. 4B illustrates dose-dependent inhibition by compounds 18 and 10, as well as a related triazolothiadiazine control that possesses no PDE4 inhibition (30 min, mean±s.d. from independent triplicates). The percentage of PKA activation was normalized based upon 20 (1 μM) pretreated cells. FIG. 4C illustrates the real-time kinetics of inhibition by compound 10 (10 μM, four independent samples) (normalized to the control experiment involving pretreatment with 1 μM 20).

FIG. 5A-B shows a schematic model of PDE4B complexed with compound 10 of the invention. The left panel details the entire PDE4B structure (N-terminal domain, a catalytic domain and a C-terminal domain) bound to compound 10. The right panel shows the catalytic domain bound to compound 10 including interactions with conserved glutamine (Q443) isoleucine (I410) and phenylalanine (F446) and the Zn²⁺ (grey) and Mg²⁺ (green) cations.

### DETAILED DESCRIPTION OF THE INVENTION

The invention generally relates to phosphodiesterase inhibitors, for example, phosphodiesterase 4 inhibitors. Such inhibitors are useful for treating and inhibiting a number of diseases and disorders. For example, the phosphodiesterase inhibitors of the invention can be used for treating and inhibiting inflammation, asthma, bronchitis, chronic obstructive pulmonary disease, inflammatory bowel disease, depression, psychosis and memory loss. Thus, the present compounds can relieve the symptoms of inflammation, asthma, bronchitis, chronic obstructive pulmonary disease, inflammatory bowel disease, depression, psychosis and improve memory.

### DEFINITIONS

As used herein a phosphodiesterase inhibitor is a compound or drug that blocks one or more of the five subtypes of the enzyme phosphodiesterase (PDE), therefore preventing the inactivation of the intracellular second messengers, cyclic adenosine monophosphate (cAMP) and cyclic guanosine monophosphate (cGMP), by the respective PDE subtype(s).

As used herein, a phosphodiesterase 4 (PDE4) inhibitor is a compound or drug that specifically inhibits PDE4. In some embodiments, the PDE4 inhibitor inhibits PDE4 at about a 2-fold, or 5-fold or 10-fold lower concentration than the PDE4 inhibitor inhibits PDE1, PDE3, PDE5, PDE7, PDE9, PDE10 and/or PDE11 enzymes.

### Phosphodiesterases

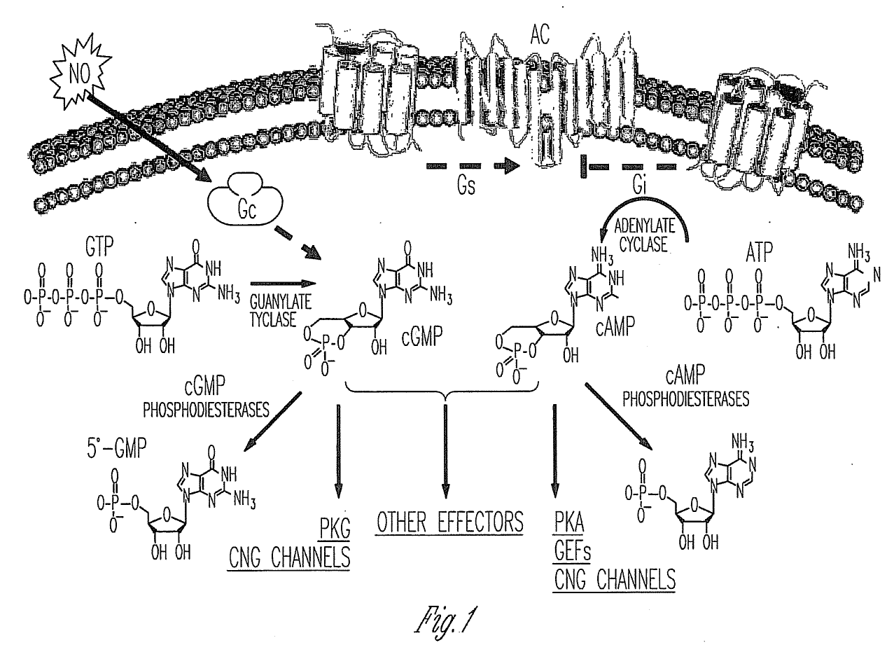

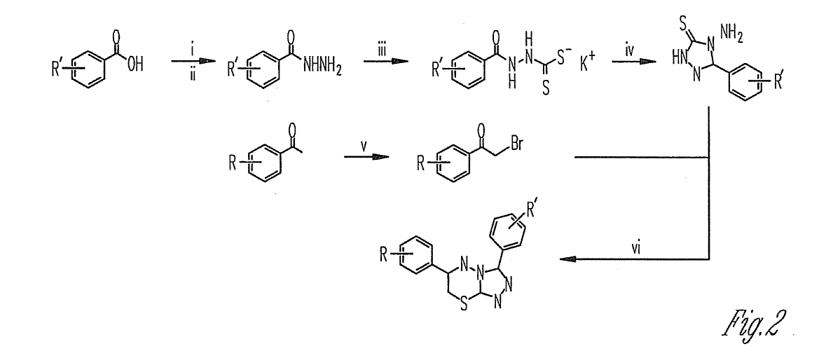

Cyclic 3′, 5′ adenosine monophosphate (cAMP) is a second messenger that mediates the actions of numerous cellular receptors, and is a key element in the regulation of cell signaling and gene transcription. Beavo et al., Nat. Rev. Mol. Cell. Biol. 2002, 3, 710; Johannessen et al., Cellular Signaling 2004, 16, 1211. The control of intracellular cAMP levels is accomplished by a balance of cAMP synthesis by adenylate cyclase, and its degradation (hydrolysis) by a variety of phosphodiesterases (PDEs) (see, FIG. 1). Cyclic guanosine monophosphate (cGMP) is controlled by similar mechanisms.

The presence of these cyclic nucleotides have regulatory effects on protein kinase A (PKA), protein kinase G (PKG), the guanine-nucleotide exchange factors (GEFs), and the cyclic-nucleotide gated (CNG) sodium and calcium channels. The production of cAMP by adenylate cyclase (AC) and the degradation of cAMP by phosphodiesterases (PDEs) are highly regulated. Manipulation of cAMP and cGMP levels in the cell represents a powerful mechanism for controlling cellular physiology. Small molecules modulators of adenylate cyclase, guanylate cyclase, and phosphodiesterases are utilized as both research tools and as clinically used drugs. Menniti et al., Nat Rev Drug Discov. 2006, 5, 660.

The phosphodiesterase (PDE) class of enzymes contains eleven principal isozymes (designated PDE1-PDE11) with twenty-one characterized gene products. Bender et al., Pharmacol. Rev. 2006, 58, 488. The PDE4 family is comprised of 4 primary gene products (PDE4A, PDE4B, PDE4C, PDE4D) and is highly expressed in neutrophils and monocytes, CNS tissue and smooth muscles of the lung. The PDE4 gene family is of particular interest because of its role in inflammation and a variety of other disorders and diseases. McKenna & Muller, In Beavo et al., Eds., CYCLIC NUCLEOTIDE PHOSPHODIESTERASES IN HEALTH AND DISEASE, pp 667, (CRC Press: 2006); Zhang et al., Expert Opin. Ther. Targets 2005, 9, 1283; Huang et al., Curr. Opin. Chem. Biol. 2001, 5, 432; Souness et al., Immunopharmacol. 2000, 47, 127.

PDE4 inhibitors are useful for treating a variety of diseases and disorders. For example, PDE4 inhibitors can be used to treat diseases and disorders such as asthma, chronic obstructive pulmonary disease (COPD), memory problems and inflammatory conditions. McKenna & Muller, In Beavo et al., Eds., CYCLIC NUCLEOTIDE PHOSPHODIESTERASES IN HEALTH AND DISEASE, pp 667, (CRC Press: 2006); Zhang et al., Expert Opin. Ther. Targets 2005, 9, 1283; Dyke, H. J. Expert Opin. Ther. Patents 2007, 17, 1183; Schmidt et al., Br. J. Pharmacol. 2000, 131, 1607. PDE4 also has a role in memory and depressive disorders, as well as inflammatory bowel disease. Tully et al., J. Nat. Rev. Drug Discov. 2003, 2, 267; Keshavarizian et al., Expert Opin. Investig. Drugs 2007, 16, 1489.

Due to the wide-ranging therapeutic interest in PDE4, certain compounds capable of potent and selective PDE4 have been developed, including the PDE4 inhibitors have entered into clinical evaluation including rolipram (1; Kanes et al., Neuroscience, 2007, 144, 239), roflumilast (2; Boswell-Smith & Page, Expert Opin. Investig. Drugs 2006, 15, 1105), cilomilast (3; Kroegel & Foerster, Expert Opin. Investig. Drugs 2007, 16, 109), tofimilast (4; Duplantier et al., J. Med. Chem. 2007, 50, 344). The structures of some of these compounds are compared to a compound of the invention (5) below.

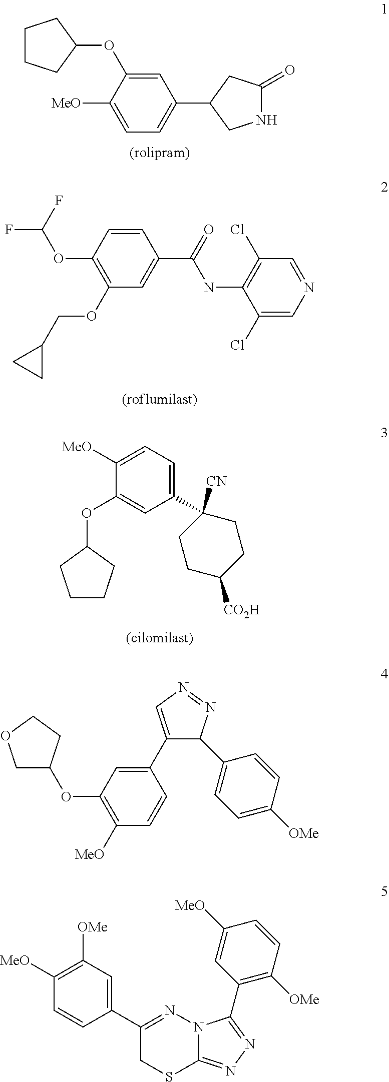

Cilomilast (3) may be approved for use in maintenance of lung function in COPD, but is still under study due to prevalent adverse effects upon the gastrointestinal system (nausea/vomiting and abdominal pain). Zhang et al., Expert Opin. Ther. Targets 2005, 9, 1283. The potentially important clinical benefits of PDE4 inhibition, coupled with the limitations of current PDE4 inhibitors, highlight the need for novel PDE4 inhibitors with fewer side effects.

The invention is therefore directed to a novel class of phosphodiesterase inhibitors.

### Phosphodiesterase Inhibitors

High-throughput screening was used to identify small molecule compounds that modulate biochemical or cellular processes by employing the NIH Molecular Libraries Initiative (MLI), which has made available public sector screening, cheminformatics, and chemistry efforts on a large scale. Austin et al., Science 2004, 306, 1138. Several substituted 3,6-diphenyl-7H-[1,2,4]triazolo[3,4-b][1,3,4]thiadiazine and 6-(3,4-dimethoxyphenyl)-3-(2-methoxyphenyl)-7,8-dihydro-[1,2,4]triazolo[4,3-b]pyridazine compounds have been identified as potent inhibitors of PDE4.

Examples of phosphodiesterase inhibitors of the invention include those of formula I:

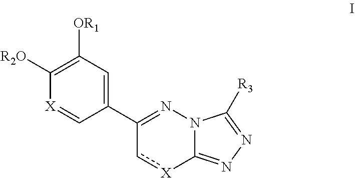

wherein:
   X is CH, CH₂, or heteroatom;
   each R₁ and R₂ is separately alkyl, haloalkyl, cycloalkyl, cycloalkylhalo, heterocycloalkyl, or aryl, where the alkyl, cycloalkyl, cycloalkylhalo, heterocycloalkyl, or aryl can be covalently linked to the oxygen via a lower alkyl; and
   R₃ is aryl substituted with 1-3 alkyl, alkenyl, alkynyl, cycloalkyl, cycloalkenyl, aryl, heteroaryl, OH, O-alkyl, SH, S-alkyl, NH₂, NH-alkyl, N-dialkyl, NH-acyl, NH-aryl, OCO-alkyl, SCO-alkyl, SOH, SO-alkyl, SO₂H, SO₂-alkyl, SO₂NH₂, SO₂NH-alkyl, SO₂N-dialkyl, CF₃, F, Cl, Br, or I groups.

In some embodiments, the X heteroatom is O, S, N or NH.

For example, the compound can have one of the following formulae:

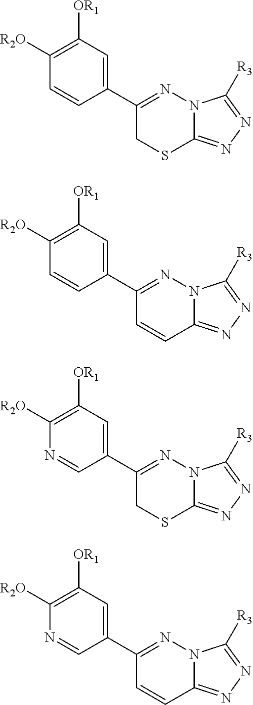

wherein:
   each R₁ and R₂ is separately alkyl, haloalkyl, cycloalkyl, cycloalkylhalo, heterocycloalkyl, or aryl, where the alkyl, cycloalkyl, cycloalkylhalo, heterocycloalkyl, or aryl can be covalently linked to the oxygen via a lower alkyl;
   R₃ is aryl substituted with 1-3 alkyl, alkenyl, alkynyl, cycloalkyl, cycloalkenyl, aryl, heteroaryl, OH, O-alkyl, SH, S-alkyl, NH₂, NH-alkyl, N-dialkyl, NH-acyl, NH-aryl, OCO-alkyl, SCO-alkyl, SOH, SO-alkyl, SO₂H, SO₂-alkyl, SO₂NH₂, SO₂NH-alkyl, SO₂N-dialkyl, CF₃, F, Cl, Br, or I groups.

In other embodiments, the X can be N or CH in the following ring:

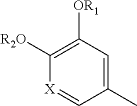

In other embodiments, the X can be S or CH in the following ring:

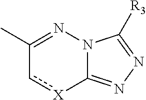

The R₃ moiety in the compounds of the invention can be an aryl, for example, a phenyl or naphthyl group. In some embodiments, the R₃ aryl group is a phenyl group. The R₃ aryl group is substituted with 1-3 alkyl, alkenyl, alkynyl, cycloalkyl, cycloalkenyl, aryl, heteroaryl, OH, O-alkyl, SH, S-alkyl, NH₂, NH-alkyl, N-dialkyl, NH-acyl, NH-aryl, OCO-alkyl, SCO-alkyl, SOH, SO-alkyl, SO₂H, SO₂-alkyl, SO₂NH₂, SO₂NH-alkyl, SO₂N-dialkyl, CF₃, F, Cl, Br, or I groups. Thus, halide atoms such as Br, Cl, F and I atoms can be present on the R₃ aryl group. Alkyl groups present on the R₃ aryl are typically lower alkyl groups, for example, methyl or ethyl. When R₃ is phenyl, for example, the phenyl can have 1-3 lower alkyl, lower alkoxy, lower cycloalkyl or lower alkylhalide groups. However, in some embodiments, the R₃ phenyl group is substituted with 2 lower alkyl, lower alkoxy or lower alkylhalide groups. One example of an R₃ group that gives rise to highly potent phosphodiesterase-4 inhibitors is dimethoxyphenyl. For example, R₃ can be dimethoxyphenyl, where the two methoxy residues are para, meta or ortho to one another. In some embodiments where R₃ is dimethoxyphenyl, the two methoxy residues are para to one another. Thus, for example, the compounds of the invention can have an R₃ group with the following structure:

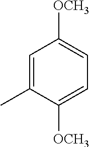

The R₁ and R₂ groups are separately alkyl, haloalkyl, cycloalkyl, cycloalkylhalo, heterocycloalkyl, or aryl, where the alkyl, cycloalkyl, cycloalkylhalo, heterocycloalkyl, or aryl can be covalently linked to the oxygen via a lower alkyl. The R₁ and R₂ alkyl groups can in some cases each be lower alkyl, for example, ethyl or methyl. However, other highly effective compounds have cycloalkyl or heterocycloalkyl moieties in the R₁ and R₂ groups, where the cycloalkyl or heterocycloalkyl moieties can be directly attached to the oxygen or linked to the oxygen by a lowere alkyl group. The R₁ and R₂ haloalkyl groups or cycloalkylhalo groups can lower alkyl or lower cycloalkyl groups that are substituted with 1-3 halide atoms. Halide atoms such as Br, Cl, F and I atoms can be used for the R₁ and R₂ haloalkyl groups or cycloalkylhalo groups.

For example, one or more of the following compounds can be used in the practice of the invention:

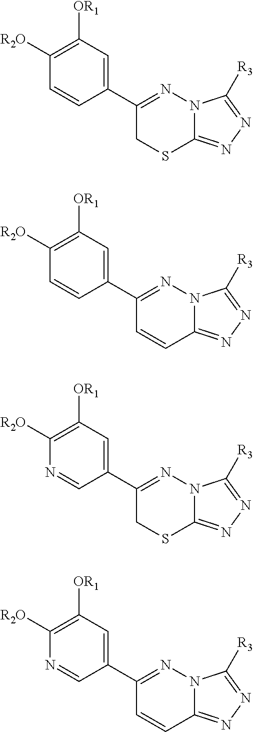

wherein each R₁, R₂ and R₃ is as described above. Examples of compounds that can be used in the practice of the invention include those with the following formulae:

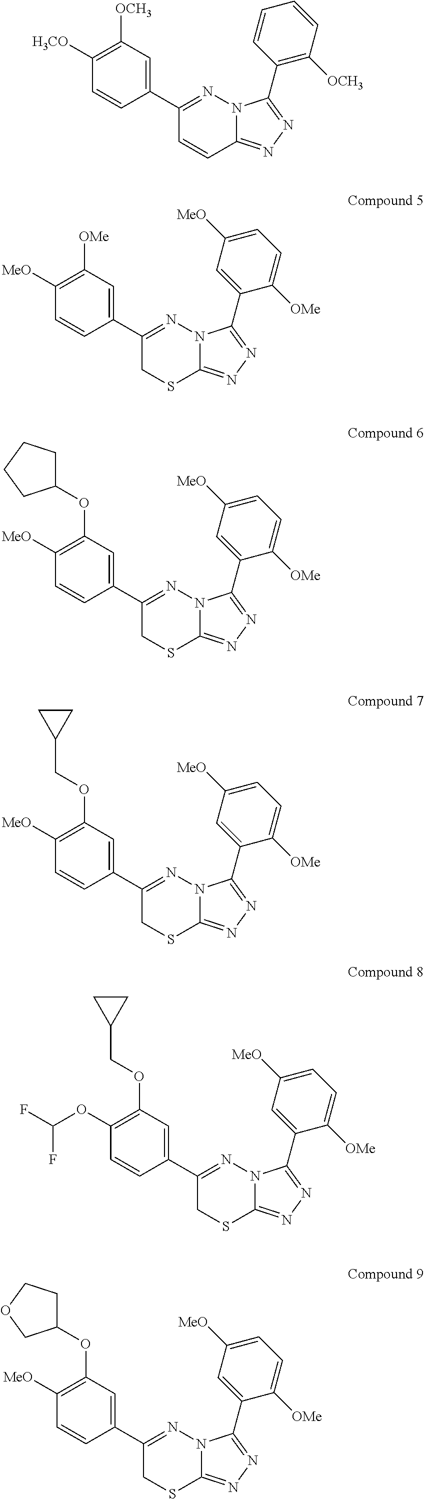

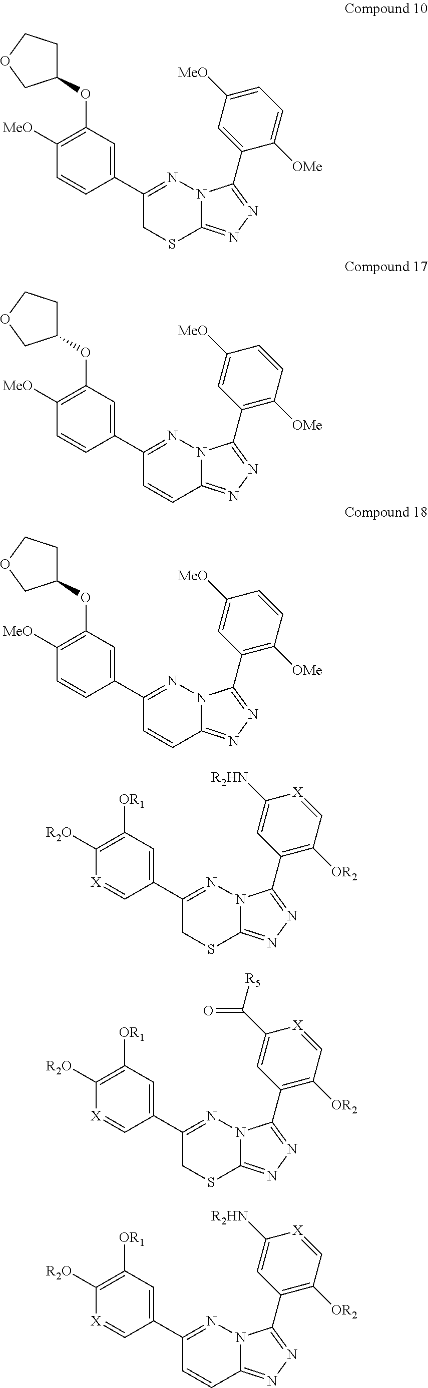

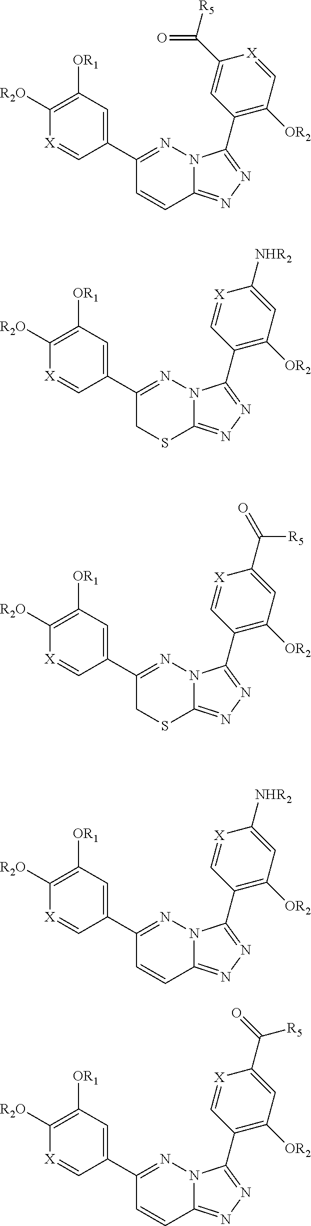

wherein:
   X is CH or heteroatom;
   each R₁ and R₂ is separately alkyl, haloalkyl, cycloalkyl, cycloalkylhalo, heterocycloalkyl, or aryl, where the alkyl, cycloalkyl, cycloalkylhalo, heterocycloalkyl, or aryl can be covalently linked to the oxygen via a lower alkyl; and
   R₅ is amide, ester, alkyl or aryl.

As illustrated herein, these compounds are capable of selective inhibition of PDE4. For example, the compounds of the invention are effective inhibitors of PDE4 at low concentrations, such as about 0.1 nanomolar to 1500 nanomolar concentrations, or at about 1 nanomolar to 1000 nanomolar concentrations, or at about 5 nanomolar to 750 nanomolar concentrations, or at about 10 nanomolar to 500 nanomolar concentrations.

For example, compounds 5 and 18 of the invention exhibit 50% inhibition of various PDE4 isoforms at concentrations as low as about 0.1 nanomolar to about 150 nanomolar, as shown below.

5

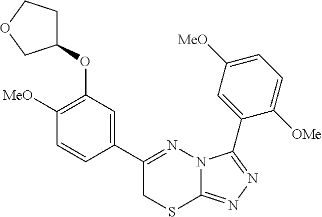

| PDE Type | Compound 5 |
|---|---|
| PDE4A1A | 0.26 nM |
| PDE4B1 | 2.3 nM |
| PDE4B2 | 1.6 nM |
| PDE4C1 | 46 nM |
| PDE4D2 | 1.9 nM |

18

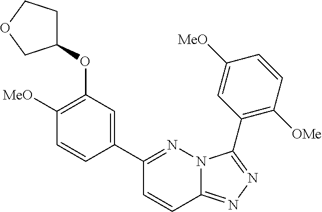

| PDE Type | Compound 18 |
|---|---|
| PDE4A1A | 0.6 nM |
| PDE4B1 | 4.1 nM |
| PDE4B2 | 2.9 nM |
| PDE4C1 | 106 nM |
| PDE4D2 | 2.1 nM |

Thus, desirable compounds of the present compounds can have an extended phenyl ring attached at the 3 position of the 1,2,4-triazole. Desirable compounds can also have a ring fused to the triazole, which can contain nitrogen, sulfur and/or oxygen heteroatoms. In some embodiments it is desirable to have two substituents on the left phenyl group that are in the ortho positions relative to each other, thereby forming a catechol diether moiety. According to the invention, the catechol diether moiety interacts with the conserved glutamine residue, and the use of molecular modeling and available structural information for both isoforms of PDE4 design of novel analogues that favor individual PDE4 isoforms.

In summary, a novel class of PDE4 inhibitors has been identified that are based upon a 3,6-diphenyl-7H-[1,2,4]triazolo[3,4-b][1,3,4]thiadiazine and 6-(3,4-dimethoxyphenyl)-3-(2-methoxyphenyl)-7,8-dihydro-[1,2,4]triazolo[4,3-b]pyridazine core structures. Initial results indicate that these compounds are among the catechol diether class of PDE4 inhibitors. Some of the most potent compounds of the invention have a 3,4-dimethoxy functions on a phenyl moiety located at the 5 position of the 3,6-dihydro-2H-1,3,4-thiadiazine or pyridazine ring but not the phenyl ring attached at the 3 position of the 1,2,4-triazole.

### Therapeutic Uses

According to the invention, the PDE4 inhibitors are useful for treating and/or inhibiting inflammatory, neuropsychiatric and immunologic diseases and disorders. Not only are the present inhibitors small molecules that can selectively inhibit PDE4 isotypes. Moreover, the inhibitors of the invention exhibit some preference for PDE4B over PDE4D. Such selectivity is extremely useful. For example, PDE4B knockout animal models exhibit anxiety (i.e., anxiogenic phenotypes; see Zhang et al., Neuropsychopharmacology 33: 1611-23 (2008). Moreover, mutations in PDE4B-specific binding sites of DISC1 affect its binding to PDE4B and confer phenotypes related to schizophrenia and depression (see, e.g., Murdoch et al., J. Neurosci. 2007, 27, 9513). Down-regulation of PDE4A and PDE4B are correlated with suppression of inflammatory cell function (see, e.g., Manning et al., Br. J. Pharmacol. 1999, 128, 1393). By contrast, PDE4D is thought to play a role in vomiting (emesis) (Zhang et al., Expert Opin. Ther. Targets 2005, 9, 1283).

In view of their PDE-inhibiting properties, the compounds of the invention can be employed in human and veterinary medicine as therapeutics, where they can be used, for example, for the treatment and prophylaxis of the following illnesses: acute and chronic (in particular inflammatory and allergen-induced) airway disorders of varying origin (bronchitis, allergic bronchitis, bronchial asthma, emphysema, COPD); dermatoses (especially of proliferative, inflammatory and allergic type) such as psoriasis (vulgaris), toxic and allergic contact eczema, atopic eczema, seborrhoeic eczema, Lichen simplex, sunburn, pruritus in the anogenital area, alopecia areata, hypertrophic scars, discoid lupus erythematosus, follicular and widespread pyodermias, endogenous and exogenous acne, acne rosacea and other proliferative, inflammatory and allergic skin disorders; disorders which are based on an excessive release of TNF and leukotrienes, for example disorders of the arthritis type (rheumatoid arthritis, rheumatoid spondylitis, osteoarthritis and other arthritic conditions), disorders of the immune system (AIDS, multiple sclerosis), graft versus host reaction, allograft rejections, types of shock (septic shock, endotoxin shock, gram-negative sepsis, toxic shock syndrome and ARDS (adult respiratory distress syndrome)) and also generalized inflammations in the gastrointestinal region (Crohn's disease and ulcerative colitis); disorders which are based on allergic and/or chronic, immunological false reactions in the region of the upper airways (pharynx, nose) and the adjacent regions (paranasal sinuses, eyes), such as allergic rhinitis/sinusitis, chronic rhinitis/sinusitis, allergic conjunctivitis and also nasal polyps; but also disorders of the heart which can be treated by PDE inhibitors, such as cardiac insufficiency, or disorders which can be treated on account of the tissue-relaxant action of the PDE inhibitors, such as, for example, erectile dysfunction or colics of the kidneys and of the ureters in connection with kidney stones. In addition, the compounds of the invention are useful in the treatment of diabetes insipidus and conditions associated with cerebral metabolic inhibition, such as cerebral senility, senile dementia (Alzheimer's disease), memory impairment associated with Parkinson's disease or multiinfarct dementia; and also illnesses of the central nervous system, such as depressions or arteriosclerotic dementia.

The invention further relates to a method for the treatment of mammals, including humans, who are suffering from, or who may soon be suffering from, one of the abovementioned illnesses. The method is characterized in that a therapeutically active and pharmacologically effective and tolerable amount, of one or more of the compounds according to the invention is administered to the mammal, particularly a mammal suffering from or soon may be suffering from, one of the abovementioned illnesses.

The invention further relates to the compounds according to the invention for use in the treatment and/or prophylaxis of illnesses, especially the illnesses mentioned.

The invention also relates to the use of the compounds according to the invention for the production of medicaments which are employed for the treatment and/or prophylaxis of the illnesses mentioned.

The invention furthermore relates to medicaments for the treatment and/or prophylaxis of the illnesses mentioned, which contain one or more of the compounds according to the invention.

### Compound Synthesis

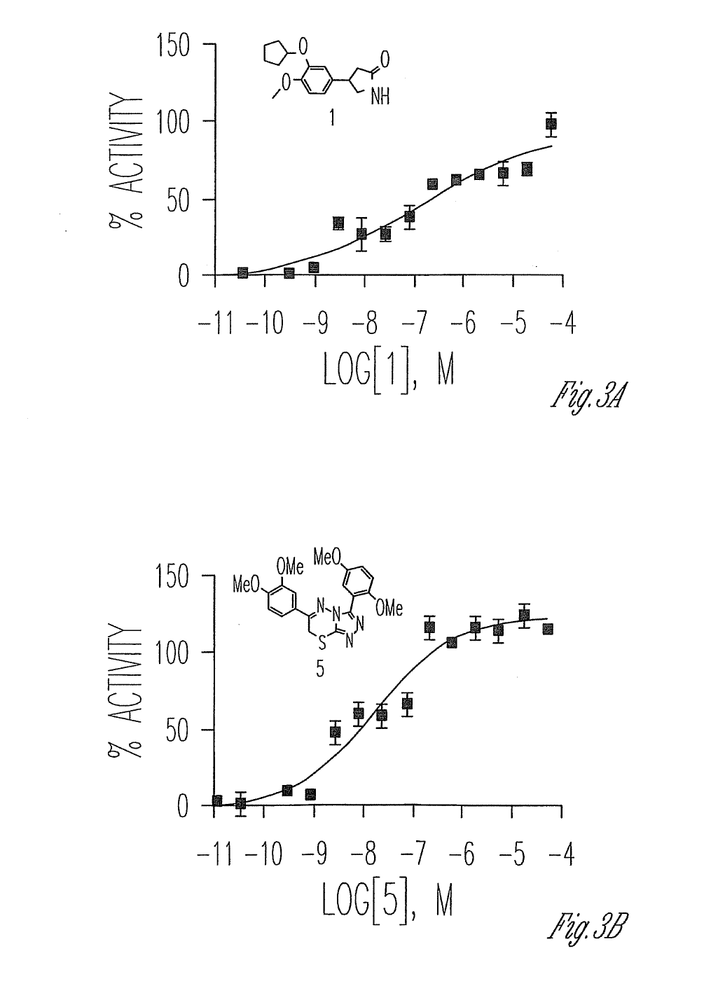

The compounds of the invention can be synthesized using any available procedures available to one of skill in the art. For example, the compounds can be synthesized via procedures described in the literature to construct the heterocyclic framework (FIG. 2). Procedures that may be helpful in the synthesis of the compounds of the invention include those described in Pollak & Ti\check{s}ler, Tetrahedron 1966, 22, 2073-2079; Albright et al., J. Med. Chem. 1981, 24, 592-600; Carling et al., J. Med. Chem. 2005, 48, 7089-7092; Swamy et al., Struct. Chem. 2006, 17, 91; Reid et al., J. Heterocyclic Chem. 1976, 13, 925; Jacob & Nichols, D. E. J. Med. Chem. 1981, 24, 1013; and Moreno et al., Eur. J. Org. Chem. 2002, 13, 2126.

Briefly, substituted benzoic acids were transformed into their analogous methyl esters (by reaction with methanol in acid) and then into substituted benhydrazides (by refluxing with hydrazine in ethanol). In some embodiments, compounds without sulfur in the ring were made and in other instances, compounds with sulfur substituents in the ring were made. To form carbodithioates, the hydrazides were treated with an ethanolic solution of potassium hydroxide to which carbon disulfide was added. The dithioates were heated to about 105° C. to 125° C. (e.g., 113° C.) with hydrazine monohydrate and water, then cooled and acidified to provide the substituted triazole. Good yields were obtained. When necessary, α-bromoketones were produced upon treatment of the corresponding acetophenones with bromine in chloroform. Modest to good yields of the α-bromoketones were obtained. Condensation between the substituted triazole and substituted α-bromoketones was effected by heating in ethanol.

For example, condensation between appropriately substituted 2-bromo-1-phenylethanone (ultimately the phenyl ring at the C6 position of the heterocycle) and appropriately substituted 4-amino-3-phenyl-1H-1,2,4-triazole-5(4H)-thione (ultimately the phenyl ring at the C3 position of the heterocycle) was accomplished in ethanol at elevated temperatures. To incorporate the cyclopentyloxy, cyclopropylmethoxy, 2-difluoromethoxy and O-3-tetrahydrofuranyl moieties unto the 2-bromo-1-phenylethanone precursor, the compound 1-(3-hydroxy-4-methoxyphenyl)ethanone was used as an orthogonally protected starting reagent. For the cyclopentyloxy and cyclopropylmethoxy substituents, nucleophilic displacement of the corresponding alkyl bromides was used to ultimately provide the substitution pattern found, for example, in compounds 6 and 7. Reaction of 1-(3-(cyclopentyloxy)-4-methoxyphenyl)ethanone with dodecane-1-thiol in sodium methoxide/DMF at 100° C. provided demethylation in a mild manner (see, Katoh et al., Synlett 2005, 19, 2919-2922). Treatment of the resulting 1-(3-(cyclopentyloxy)-4-hydroxyphenyl)ethanone with sodium 2-chloro-2,2-difluoroacetate in DMF at 100° C. afforded the incorporation of the 2-difluoromethoxy functionality on the C4 position of the catachol moiety (found in compound 8) (see, Hall et al., Bioorg. Med. Chem. Lett. 2007, 17, 916-920). Mitsonobu conditions were utilized to condense tetrahydrofuran-3-ol (both racemic and R) with 1-(3-hydroxy-4-methoxyphenyl)ethanone to provide compounds 9 and 10.

The synthesis of substituted 7H-[1,2,4]triazolo[3,4-b][1,3,4]thiadiazines was accomplished as shown in FIG. 2.

The synthesis of [1,2,4]triazolo[4,3-b]pyridazines was based upon the precedented works of (Pollak & Ti\check{s}ler, Tetrahedron 1966, 22, 2073-2079; Albright et al., J. Med. Chem. 1981; 24, 592-600; Carling et al., J. Med. Chem. 2005, 48, 7089-7092). The general method is outlined in Scheme 1 below and begins with the coupling of commercially available 2,5-dimethoxybenzoic acid (11) and 3-chloro-6-hydrazinylpyridazine (12) to provide N-(6-chloropyridazin-3-yl)-2,5-dimethoxybenzohydrazide (13) in good yields. Direct treatment of 13 with POCl₃ at elevated temperature afforded the cyclization to form the core [1,2,4]triazolo[4,3-b]pyridazine ring system (compound 14) and provided a compound intermediate for entry into the convergent syntheses of multiple products via end-stage Suzuki-Miyaura couplings. Boronic acids 15 and 16 were synthesized independently utilizing the aforementioned Mitsonobu protocols and displacement of an aryl bromide with boronic acid. Following purification of 15 and 16, standard Suzuki-Miyaura conditions with microwave irradiation produced the appropriately substituted [1,2,4]triazolo[4,3-b]pyridazines 17 and 18 in good yields.

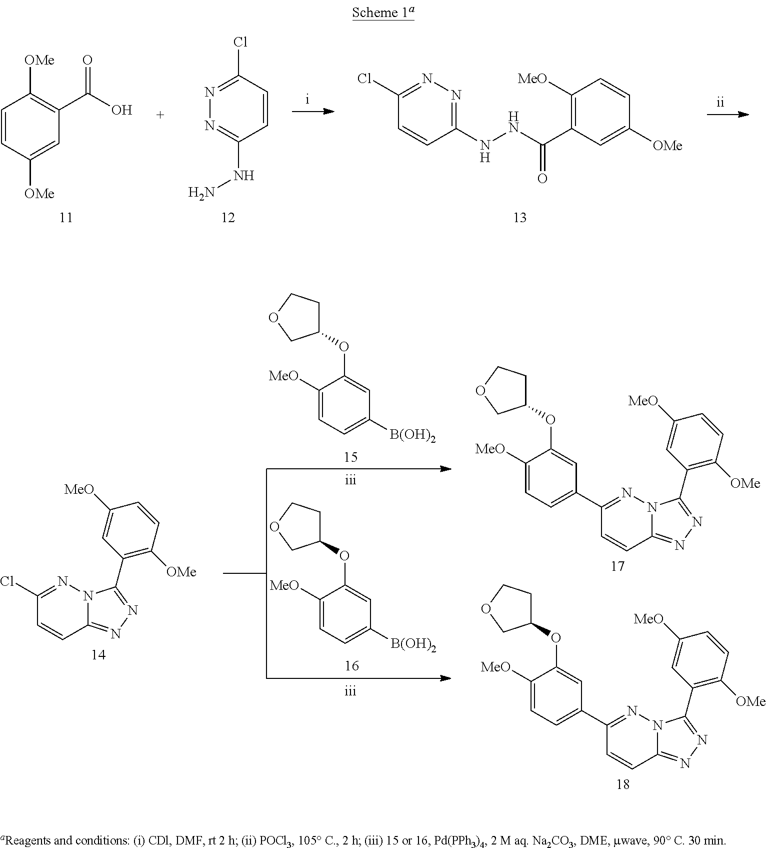

Further details on synthetic procedures are provided in the Examples.

### Therapeutic Administration

The compounds (“therapeutic agents”) of the invention are administered so as to achieve a reduction in at least one symptom associated with a disease or disorder associated with PDE4 activity.

To achieve the desired effect(s), the compound, or a combination of compounds, may be administered as single or divided dosages, for example, of at least about 0.0001 mg/kg to about 500 mg/kg, of at least about 0.001 mg/kg to about 300 mg/kg, of at least about 0.01 mg/kg to about 100 mg/kg, or of at least about 0.1 mg/kg to about 50 mg/kg of body weight, although other dosages may provide beneficial results. The amount administered will vary depending on various factors including, but not limited to, the inactivated viral agent chosen, the disease, the weight, the physical condition, the health, the age of the mammal, or whether prevention or treatment is to be achieved. Such factors can be readily determined by the clinician employing animal models or other test systems that are available in the art.

Administration of the therapeutic agents in accordance with the present invention may be in a single dose, in multiple doses, in a continuous or intermittent manner, depending, for example, upon the recipient's physiological condition, whether the purpose of the administration is therapeutic or prophylactic, and other factors known to skilled practitioners. The administration of certain compounds and therapeutic agents of the invention can be intermittent over a preselected period of time, for example, in a series of spaced doses. Both local and systemic administration is contemplated.

To prepare the composition, compounds are prepared according to the methods described herein, or those available in the art, and purified as necessary or desired. In some embodiments, the compounds can be lyophilized and/or stabilized. The selected compound(s) can then be adjusted to the appropriate concentration, and optionally combined with other agents.

The absolute weight of a given compound included in a unit dose can vary widely. For example, about 0.01 to about 2 g, or about 0.1 to about 500 mg, of at least one compound of the invention, or a plurality of compounds, can be administered. Alternatively, the unit dosage can vary from about 0.0001 g to about 5 g, from about 0.001 g to about 3.5 g, from about 0.01 g to about 2.5 g, from about 0.1 g to about 1 g, from about 0.1 g to about 0.8 g, from about 0.1 g to about 0.4 g, or from about 0.1 g to about 0.2 g.

One or more suitable unit dosage forms comprising the therapeutic agents of the invention can be administered by a variety of routes including oral, parenteral (including subcutaneous, intravenous, intramuscular and intraperitoneal), rectal, dermal, transdermal, intrathoracic, intrapulmonary and intranasal (respiratory) routes. The therapeutic agents may also be formulated for sustained release (for example, using microencapsulation, see WO 94/07529, and U.S. Pat. No. 4,962,091). The formulations may, where appropriate, be conveniently presented in discrete unit dosage forms and may be prepared by any of the methods well known to the pharmaceutical arts. Such methods may include the step of mixing the compounds with liquid carriers, solid matrices, semi-solid carriers, finely divided solid carriers or combinations thereof, and then, if necessary, introducing or shaping the product into the desired delivery system.

When the therapeutic agents are prepared for oral administration, they are generally combined with a pharmaceutically acceptable carrier, diluent or excipient to form a pharmaceutical formulation, or unit dosage form. For oral administration, the compounds may be present as a powder, a granular formulation, a solution, a suspension, an emulsion or in a natural or synthetic polymer or resin for ingestion of the agents from a chewing gum. The compounds may also be presented as a bolus, electuary or paste. When orally administered the therapeutic agents of the invention can also be formulated for sustained release, e.g., the compounds can be coated, micro-encapsulated, or otherwise placed within a sustained delivery device. The total active ingredients in such formulations comprise from 0.1 to 99.9% by weight of the formulation.

By “pharmaceutically acceptable” it is meant a carrier, diluent, excipient, and/or salt that is compatible with the other ingredients of the formulation, and not deleterious to the recipient thereof.

Pharmaceutical formulations containing the therapeutic compounds can be prepared by procedures described herein and formulated using procedures known in the art using well-known and readily available ingredients. For example, the compounds can be formulated with common excipients, diluents, or carriers, and formed into tablets, capsules, solutions, suspensions, powders, aerosols and the like. Examples of excipients, diluents, and carriers that are suitable for such formulations include buffers, as well as fillers and extenders such as starch, cellulose, sugars, mannitol, and silicic derivatives. Binding agents can also be included such as carboxymethyl cellulose, hydroxymethylcellulose, hydroxypropyl methylcellulose and other cellulose derivatives, alginates, gelatin, and polyvinyl-pyrrolidone. Moisturizing agents can be included such as glycerol, disintegrating agents such as calcium carbonate and sodium bicarbonate. Agents for retarding dissolution can also be included such as paraffin. Resorption accelerators such as quaternary ammonium compounds can also be included. Surface active agents such as cetyl alcohol and glycerol monostearate can be included. Adsorptive carriers such as kaolin and bentonite can be added. Lubricants such as talc, calcium and magnesium stearate, and solid polyethyl glycols can also be included. Preservatives may also be added. The compositions of the invention can also contain thickening agents such as cellulose and/or cellulose derivatives. They may also contain gums such as xanthan, guar or carbo gum or gum arabic, or alternatively polyethylene glycols, bentones and montmorillonites, and the like.

For example, tablets or caplets containing the therapeutic agents of the invention can include buffering agents such as calcium carbonate, magnesium oxide and magnesium carbonate. Caplets and tablets can also include inactive ingredients such as cellulose, pre-gelatinized starch, silicon dioxide, hydroxy propyl methyl cellulose, magnesium stearate, microcrystalline cellulose, starch, talc, titanium dioxide, benzoic acid, citric acid, corn starch, mineral oil, polypropylene glycol, sodium phosphate, zinc stearate, and the like. Hard or soft gelatin capsules containing at least one compound of the invention can contain inactive ingredients such as gelatin, microcrystalline cellulose, sodium lauryl sulfate, starch, talc, and titanium dioxide, and the like, as well as liquid vehicles such as polyethylene glycols (PEGS) and vegetable oil. Moreover, enteric-coated caplets or tablets containing one or more of the compounds of the invention are designed to resist disintegration in the stomach and dissolve in the more neutral to alkaline environment of the duodenum.

The therapeutic agents of the invention can also be formulated as elixirs or solutions for convenient oral administration or as solutions appropriate for parenteral administration, for instance by intramuscular, subcutaneous, intraperitoneal or intravenous routes. The pharmaceutical formulations of the therapeutic agents of the invention can also take the form of an aqueous or anhydrous solution or dispersion, or alternatively the form of an emulsion or suspension or salve.

Thus, the therapeutic agents may be formulated for parenteral administration (e.g., by injection, for example, bolus injection or continuous infusion) and may be presented in unit dose form in ampoules, pre-filled syringes, small volume infusion containers or in multi-dose containers. As noted above, preservatives can be added to help maintain the shelve life of the dosage form. The compounds and/or other ingredients may form suspensions, solutions, or emulsions in oily or aqueous vehicles, and may contain formulatory agents such as suspending, stabilizing and/or dispersing agents. Alternatively, the therapeutic agents and other ingredients may be in powder form, obtained by aseptic isolation of sterile solid or by lyophilization from solution, for constitution with a suitable vehicle, e.g., sterile, pyrogen-free water, before use.

These formulations can contain pharmaceutically acceptable carriers, vehicles and adjuvants that are well known in the art. It is possible, for example, to prepare solutions using one or more organic solvent(s) that is/are acceptable from the physiological standpoint, chosen, in addition to water, from solvents such as acetone, ethanol, isopropyl alcohol, glycol ethers such as the products sold under the name “Dowanol,” polyglycols and polyethylene glycols, C₁-C₄ alkyl esters of short-chain acids, ethyl or isopropyl lactate, fatty acid triglycerides such as the products marketed under the name “Miglyol,” isopropyl myristate, animal, mineral and vegetable oils and polysiloxanes.

It is possible to add, if desired, an adjuvant chosen from antioxidants, surfactants, other preservatives, film-forming, keratolytic or comedolytic agents, perfumes, flavorings and colorings. Antioxidants such as t-butylhydroquinone, butylated hydroxyanisole, butylated hydroxytoluene and α-tocopherol and its derivatives can be added.

Also contemplated are combination products that include one or more therapeutic agents of the present invention and one or more anti-microbial agents. For example, a variety of antibiotics can be included in the pharmaceutical compositions of the invention, such as aminoglycosides (e.g., streptomycin, gentamicin, sisomicin, tobramycin and amicacin), ansamycins (e.g. rifamycin), antimycotics (e.g. polyenes and benzofuran derivatives), β-lactams (e.g. penicillins and cephalosporins), chloramphenical (including thiamphenol and azidamphenicol), linosamides (lincomycin, clindamycin), macrolides (erythromycin, oleandomycin, spiramycin), polymyxins, bacitracins, tyrothycin, capreomycin, vancomycin, tetracyclines (including oxytetracycline, minocycline, doxycycline), phosphomycin and fusidic acid.

Additionally, the therapeutic agents are well suited to formulation as sustained release dosage forms and the like. The formulations can be so constituted that they release a compound, for example, in a particular part of the intestinal or respiratory tract, possibly over a period of time. Coatings, envelopes, and protective matrices may be made, for example, from polymeric substances, such as polylactide-glycolates, liposomes, microemulsions, microparticles, nanoparticles, or waxes. These coatings, envelopes, and protective matrices are useful to coat indwelling devices, e.g., stents, catheters, peritoneal dialysis tubing, draining devices and the like.

For topical administration, the compounds may be formulated as is known in the art for direct application to a target area. Forms chiefly conditioned for topical application take the form, for example, of creams, milks, gels, dispersion or microemulsions, lotions thickened to a greater or lesser extent, impregnated pads, ointments or sticks, aerosol formulations (e.g., sprays or foams), soaps, detergents, lotions or cakes of soap. Other conventional forms for this purpose include wound dressings, coated bandages or other polymer coverings, ointments, creams, lotions, pastes, jellies, sprays, and aerosols. Thus, the therapeutic agents of the invention can be delivered via patches or bandages for dermal administration. Alternatively, the therapeutic agents can be formulated to be part of an adhesive polymer, such as polyacrylate or acrylate/vinyl acetate copolymer. For long-term applications it might be desirable to use microporous and/or breathable backing laminates, so hydration or maceration of the skin can be minimized. The backing layer can be any appropriate thickness that will provide the desired protective and support functions. A suitable thickness will generally be from about 10 to about 200 microns.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents. The therapeutic agents can also be delivered via iontophoresis, e.g., as disclosed in U.S. Pat. Nos. 4,140,122; 4,383,529; or 4,051,842. The percent by weight of a therapeutic agent of the invention present in a topical formulation will depend on various factors, but generally will be from 0.01% to 95% of the total weight of the formulation, and typically 0.1-85% by weight.

Drops, such as eye drops or nose drops, may be formulated with one or more of the therapeutic agents in an aqueous or non-aqueous base also comprising one or more dispersing agents, solubilizing agents or suspending agents. Liquid sprays are conveniently delivered from pressurized packs. Drops can be delivered via a simple eye dropper-capped bottle, or via a plastic bottle adapted to deliver liquid contents dropwise, via a specially shaped closure.

The therapeutic agents may further be formulated for topical administration in the mouth or throat. For example, the active ingredients may be formulated as a lozenge further comprising a flavored base, for example, sucrose and acacia or tragacanth; pastilles comprising the composition in an inert base such as gelatin and glycerin or sucrose and acacia; and mouthwashes comprising the composition of the present invention in a suitable liquid carrier.

The pharmaceutical formulations of the present invention may include, as optional ingredients, pharmaceutically acceptable carriers, diluents, solubilizing or emulsifying agents, and salts of the type that are available in the art. Examples of such substances include normal saline solutions such as physiologically buffered saline solutions and water. Specific non-limiting examples of the carriers and/or diluents that are useful in the pharmaceutical formulations of the present invention include water and physiologically acceptable buffered saline solutions such as phosphate buffered saline solutions pH 7.0-8.0.

The therapeutic agents of the invention can also be administered to the respiratory tract. Thus, the present invention also provides aerosol pharmaceutical formulations and dosage forms for use in the methods of the invention. In general, such dosage forms comprise an amount of at least one of the agents of the invention effective to treat or prevent the clinical symptoms of a specific PDE4-related disorder or disease. Any statistically significant attenuation of one or more symptoms of a disorder or disease that has been treated pursuant to the methods of the present invention is considered to be a treatment of such a disorder or disease within the scope of the invention.

Alternatively, for administration by inhalation or insufflation, the composition may take the form of a dry powder, for example, a powder mix of the therapeutic agent and a suitable powder base such as lactose or starch. The powder composition may be presented in unit dosage form in, for example, capsules or cartridges, or, e.g., gelatin or blister packs from which the powder may be administered with the aid of an inhalator, insufflator, or a metered-dose inhaler (see, for example, the pressurized metered dose inhaler (MDI) and the dry powder inhaler disclosed in Newman, S. P. in AEROSOLS AND THE LUNG, Clarke, S. W. and Davia, D. eds., pp. 197-224, Butterworths, London, England, 1984).

Therapeutic agents of the present invention can also be administered in an aqueous solution when administered in an aerosol or inhaled form. Thus, other aerosol pharmaceutical formulations may comprise, for example, a physiologically acceptable buffered saline solution containing between about 0.1 mg/ml and about 100 mg/ml of one or more of the therapeutic agents of the present invention specific for the indication or disease to be treated or prevented. Dry aerosol in the form of finely divided solid inactivated agent that are not dissolved or suspended in a liquid are also useful in the practice of the present invention. Therapeutic agents of the present invention may be formulated as dusting powders and comprise finely divided particles having an average particle size of between about 1 and 5 μm, alternatively between 2 and 3 μm. Finely divided particles may be prepared by pulverization and screen filtration using techniques well known in the art. The particles may be administered by inhaling a predetermined quantity of the finely divided material, which can be in the form of a powder. It will be appreciated that the unit content of active ingredient or ingredients contained in an individual aerosol dose of each dosage form need not in itself constitute an effective amount for treating or preventing the particular infection, indication or disease since the necessary effective amount can be reached by administration of a plurality of dosage units. Moreover, the effective amount may be achieved using less than the dose in the dosage form, either individually, or in a series of administrations.

For administration to the upper (nasal) or lower respiratory tract by inhalation, the therapeutic agents of the invention are conveniently delivered from a nebulizer or a pressurized pack or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Nebulizers include, but are not limited to, those described in U.S. Pat. Nos. 4,624,251; 3,703,173; 3,561,444; and 4,635,627. Aerosol delivery systems of the type disclosed herein are available from numerous commercial sources including Fisons Corporation (Bedford, Mass.), Schering Corp. (Kenilworth, N.J.) and American Phamoseal Co., (Valencia, Calif.). For intra-nasal administration, the therapeutic agent may also be administered via nose drops, a liquid spray, such as via a plastic bottle atomizer or metered-dose inhaler. Typical of atomizers are the Mistometer (Wintrop) and the Medihaler (Riker).

Furthermore, the compounds may also be used in combination with other therapeutic agents, for example, pain relievers, anti-inflammatory agents, antihistamines, bronchodilators and the like, whether for the conditions described or some other condition.

The present invention further pertains to a packaged pharmaceutical composition for controlling diseases or disorders such as a kit or other container. The kit or container holds a therapeutically effective amount of a pharmaceutical composition for controlling a PDE4-related disease or disorder and instructions for using the pharmaceutical composition for control of the disease or disorder. The pharmaceutical composition includes at least one compound of the present invention, in a therapeutically effective amount such that a disease or disorder is controlled.

The invention is further illustrated by the following non-limiting Examples.

### Example 1

### Materials and Methods

This Example illustrates certain methods and materials that can be used in the practice of the invention.

Cyclic nucleotide-gated cation channel assay. The PDE4 cell line (BD Biosciences, Rockville, Md.) assay was conducted as described in reference 18.

Cell culture: Cells were plated at a density of 1000 cells/well in black, clear bottom, tissue culture treated, 1536 well plates (Kalypsys, San Diego, Calif.) in 3 μL assay medium containing DMEM, 50 units/mL penicillin and 50 μg/mL streptomycin, and 2%, 5%, 10%, or 20% fetal calf serum and were incubated 12 hr at 37° C. with 5% CO₂ prior to compound screening. 3 μl/well of 1× membrane potential dye was added and incubated for 1 hr at the room temperature. 23 nL/well of compounds in DMSO solution or the positive control (1) was added with a Pintool station (Kalypsys, San Diego, Calif.).

Fluorescence assay: After 30 min room temperature incubation with compounds, the assay plate was measured in a fluorescence plate reader in the bottom reading mode (Envision, PerkinElmer) with an excitation of 535 (±20) nm and emission of 590 (±20) nm. A flying reagent dispensing (FRD) workstation (Aurora Discovery, San Diego) was used to dispense cells and reagents to 1536-well plates. The compounds were serially diluted in DMSO in 384-well plates first and reformatted into 1536-well plates at 7 μL/well using a Cybi-well dispensing station with a 384-well head (Cybio, Inc. Woburn, Mass.). A Pintool station was used to transfer 23 nL of compounds in DMSO solution to the 1536-well assay plates. The final DMSO concentration in the assay plates was under 0.5%. During compound library screening, all plate manipulations were done on an automated robotic system (Kalypsys, San Diego, Calif.). 1 was used as the positive control and data was normalized to 10 μM 1 response (100% activity). All samples were tested in duplicate.

Protein-fragmentation complementation assays. Reagents and general assay procedures and conditions were performed in a similar manner as described in Stefan et al., Proc. Natl. Acad. Sci. U.S.A. 104: 16916-16921 (2007).

Cell culture: Stable β2AR-HEK293 cells were plated into 96-well white walled microliter plates (Corning) and grown in DMEM (Invitrogen) supplemented with 10% fetal bovine serum. Transient transfections of plasmids harboring the Rluc PCA PKA reporter were performed with FuGENE-6 reagent (Roche). 48 hours following transfection, cells were treated with 19, 20, 1 (Sigma) or other compounds as indicated. The structures of compounds 1, 19 and 20 are shown below.

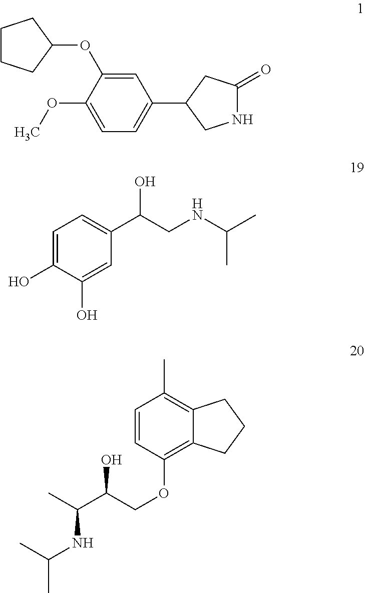

Bioluminescence assay: Immediately after treatment, exchange of medium and addition of 100 μl PBS to the 96-well white walled plates (Corning) the bioluminescence analysis was performed on an LMax™II³⁸⁴ luminometer (Molecular Devices). Rluc activities were monitored for the first 10 seconds after addition of the substrate benzyl-coelenterazine (5 μM, Nanolight).

Molecular Docking. Three-dimensional coordinates of the crystallized structure of phosphodiesterase 4B (PDE-4B) were obtained from the Protein Data Bank (PDB ID: 1XMY)(see, Card et al., Structure 12: 2233-2247 (2004)). AutoDock software version 4.0 was used for all docking simulations (Huai et al., Proc. Nat. Acad. Sci. U.S.A. 101:9624-9629 (2004)). The AutoDock Tool was applied to prepare ligands in docking format and to visualize the results. Gasteiger atomic charges were assigned and the flexibility of the molecule was determined using the AutoDock module AutoTors. All 7 torsion angles were defined so that they could be explored during the docking process. Nonpolar hydrogens, including their partial charges, were merged to parent atoms. The atomic solvation variables were assigned by the AutoDock module Addsol. Atomic interaction energy grids were calculated with the AutoDock module AutoGrid for atom probes corresponding to each atom type in the ligand. The grid box included the entire active site as observed in previous PDE4B inhibitors complexes providing sufficient space for ligand translational and rotational movement. The side chain dihedral angles of a conserved glutamine known to interact with many PDE4B inhibitors were allowed to rotate during the docking process. The Mg²⁺ and Zn²⁺ cations were included in the active site and nearby histidines were protonated accordingly. The Lamarckian genetic algorithm as implemented in AutoDock 4.0 for the docking simulations. In general, the default variables of AutoDock were used. The docked compounds were clustered into groups using an RMS deviation versus X-ray atom positions <1.0 Å. Twenty runs were executed and the most favorable free binding energy conformer was chosen for analysis. Binding constants (Kᵢ) were estimated within the AutoDock scoring function; the most favorable conformations had a Ki in the low nanomolar range.

General synthetic materials and methods. All reactions were performed under a nitrogen atmosphere passed over Drierite® (calcium sulfate) using oven-dried glassware. All commercially available reagents and solvents (anhydrous and non-anhydrous) were purchased from Aldrich (Milwaukee, Wis.), Acros (Pittsburgh, Pa.), Sigma (St. Louis, Mo.), Strem (Newburyport, Mass.), and Fisher Scientific (Fair Lawn, N.J.) and used as obtained. All reactions were stirred via a Teflon-coated stir bar on a magnetic stir-plate. Air and moisture sensitive reagents were transferred via syringe and introduced into reaction vessels through rubber septa. All microwave reactions were carried out in heavy-walled tubes containing a Teflon-coated stir bar and crimped top using an Initiator microwave (Biotage). Reaction progress was monitored by analytical TLC using 250 μm thick 60 Å silica gel plates with fluorescent indicator (Aldrich). Developed plates were visualized by UV light (254 nm) and/or treatment with PMA (phosphomolybdic acid), ninhydrin, or vanillin stain. Purification of certain compounds under acidic conditions used a Waters semi-preparative HPLC equipped with a Phenomenex Luna® C18 reverse phase (5 micron, 30×75 mm) column having a flow rate of 45 mL/min. The mobile phase was a mixture of acetonitrile and H₂O each containing 0.1% trifluoroacetic acid. Purification of certain compounds under basic conditions used a Waters semi-preparative HPLC equipped with a Phenomenex Gemini® C18 reverse phase (5 micron, 30×75 mm) column having a flow rate of 45 mL/min. The mobile phase was a mixture of acetonitrile and H₂O (0.1% NH₄OH). During purification under either acidic or basic conditions, a gradient of 20% to 60% acetonitrile over 8 minutes was used with fraction collection triggered by UV detection (220 nM). Pure fractions were concentrated and dried using Glas-Col N₂ blowdown unit at 40° C.

Melting points were determined with a MeI-Temp® capillary apparatus (Electrothermal). Infrared (IR) spectra were obtained using a Spectrum 100 FT-IR spectrometer (PerkinElmer) and reported in cm⁻¹. ¹H and ¹³C NMR spectra were recorded using an Inova 400 (100) MHz spectrometer (Varian). Chemical shifts are reported in δ (ppm) units using ¹H (residual) and ¹³C signals from CDCl₃ (7.26 and 77.23, respectively) or d₆-DMSO (2.50 and 39.51, respectively) as internal standard. Data are reported as follows: chemical shift, integration, multiplicity (s=singlet, d=doublet, t=triplet, q=quartet, m=multiplet, br=broad), coupling constant. Samples were analyzed for purity on an Agilent 1200 series LC/MS equipped with a Zorbax™ Eclipse XDB-C18 reverse phase (5 micron, 4.6×150 mm) column having a flow rate of 1.1 mL/min. The mobile phase was a mixture of acetonitrile and H₂O each containing 0.05% trifluoroacetic acid. A gradient of 5% to 100% acetonitrile over 8 minutes was used during analytical analysis. Purity of final compounds was determined to be >95%, using a 5 μL injection with quantification by AUC at 220 and 254 nM. High-resolution mass spectra (HRMS) were measured on a time-of-flight (TOF) mass spectrometer (Agilent). All yields refer to chromatographically and spectroscopically pure compounds.

Formation of Substituted Benzoates: General Procedure A: To a solution of benzoic acid (1.0 eq) in methanol (1.0M) was added sulfuric acid (catalytic). The solution was stirred at room temperature for 12 h, at which time the solvent was removed by rotary evaporation. The crude reaction mixture was partitioned between ethyl acetate and water. The aqueous layer was extracted twice with ethyl acetate and the organic extracts were combined, washed with water and brine, dried over Na₂SO₄, and concentrated by rotary evaporation. The crude product was purified by column chromatography to give the substituted benzoates in >80% yield.

Formation of Substituted Aryldithiocarbazates: General Procedure B: To a solution of methyl benzoate (1.0 eq) in ethanol (0.55M) was added hydrazine (4.0 eq). The solution was heated to reflux with stirring until TLC showed full consumption of starting materials (12 h), then cooled. The solvent was removed by rotary evaporation and the crude reaction mixture was partitioned between ethyl acetate and water. The aqueous layer was extracted twice with ethyl acetate and the organic extracts were combined, washed with water and brine, dried over Na₂SO₄, and concentrated by rotary evaporation. The crude hydrazide (1.0 eq) was taken up in ethanol (0.5M). Potassium hydroxide (1.5 eq) was added, and stirred to dissolve. To this solution, carbon disulfide (1.5 eq) was added in a drop-wise fashion. Within a period of 1-10 min, the potassium salt precipitated from solution, and was allowed to stir as a suspension for 12 h. The suspension was filtered and dried to give the potassium aryldithiocarbazates as pale yellow powders in >85%.

Formation of Substituted triazoles: General Procedure C: To a mixture of aryldithiocarbazate (1.0 eq) in water (10.0M) was added hydrazine monohydrate (2.0 eq). The mixture was heated to 113° C. to induce cyclization to the triazole with formation of hydrogen sulfide gas (reaction mixture turned greenish brown). After 0.75 h, the reaction mixture was cooled and ice chips were added. Acidification with conc. hydrochloric acid precipitated a white solid. The product was filtered and washed with 2×20 mL portions of cold water to give the triazoles. If necessary, recrystallization from 95% ethanol garnered analytically pure products. Final yields ranged from 75-90%.

Formation of Substituted 2-bromoacetophenones: General Procedure D: To a solution of substituted acetophenone in chloroform (0.35M) was added bromine (1.2 eq). The solution was stirred at room temperature for 0.5 h, then heated to reflux for another 0.5-2 h until TLC showed full consumption of starting materials. The reaction mixture was concentrated by rotary evaporation and the crude product was purified by column chromatography. Final yields ranged from 50-95%.

Formation of Substituted 3,6-diphenyl-7H-[1,2,4]-triazolo[3,4-b][1,3,4]thiadiazines: General Procedure E: To a mixture of triazole (1.0 eq) and substituted 2-bromoacetophenone (1.0 eq) was added ethanol (0.1M). The reaction mixture was sealed in a crimp-top high pressure vessel and stirred at 105° C. for 4 h. The crude reaction mixture was partitioned between methylene chloride and water. The aqueous layer was removed and the organic layer was washed with a mixture of water and brine, then concentrated by rotary evaporation. The crude product was purified by semi-preparative HPLC.

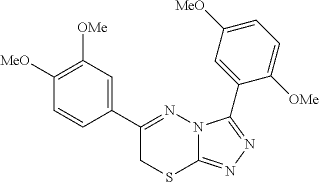

3-(2,5-dimethoxyphenyl)-6-(3,4-dimethoxyphenyl)-7H-[1,2,4]triazolo[3,4-b]-[1,3,4]thiadiazine (5). yellow oil. ¹H NMR (CDCl₃, 400 MHz) δ 7.43 (d, 1H, J=2.0 Hz), 7.40 (dd, 1H, J=2.0, 8.4 Hz), 7.23 (d, 1H, J=3.2 Hz), 7.06 (dd, 1H, J=3.2, 9.2 Hz), 6.93 (dd, 2H, J=3.2, 12.0 Hz), 4.04 (s, 3H), 3.93 (s, 3H), 3.84 (s, 3H), 3.79 (s, 3H), 3.69 (s, 3H). ¹³C NMR (CDCl₃, 100 MHz) δ 153.3, 152.4, 152.2, 152.1, 151.6, 149.3, 141.4, 126.0, 121.1, 117.7, 116.3, 115.9, 112.6, 110.5, 109.2, 56.4, 56.0, 55.9, 55.8, 23.2. LC/MS: RT (min)=5.06; (MH⁺) 413.1. HRMS: (CI+, m/z), calcd for C₂₀H₂₁N₄O₄S (MH⁺), 413.1205; found, 413.1289.

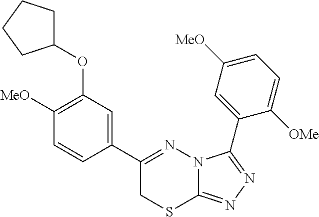

6-(3-(cyclopentyloxy)-4-methoxyphenyl)-3-(2,5-dimethoxyphenyl)-7H-[1,2,4]triazolo[3,4-b][1,3,4]thiadiazine (6). pale yellow oil. ¹H NMR (CDCl₃, 400 MHz) δ 7.44 (d, 1H, J=2.0 Hz), 7.31 (dd, 1H, J=2.2, 8.4 Hz), 7.22 (d, 1H, J=3.1 Hz), 7.05 (dd, 1H, J=3.1, 9.2 Hz), 6.94 (d, 1H, J=9.2 Hz), 6.89 (d, 1H, J=8.2 Hz), 4.68-4.72 (m, 1H), 3.97 (s, 2H), 3.90 (s, 3H), 3.81 (s, 3H), 3.70 (s, 3H) 1.79-1.89 (m, 6H), 1.57-1.61 (m, 2H). ¹³C NMR (CDCl₃, 100 MHz) δ 153.6, 153.3, 152.7, 152.2, 148.1, 141.6, 125.5, 120.9, 118.0, 116.4, 115.2, 112.7, 112.4, 110.9, 80.7, 56.3, 56.1, 55.9, 32.7, 24.1, 22.9. LC/MS: RT (min)=5.95; (MH⁺) 467.1. HRMS: (CI+, m/z), calcd for C₂₄H₂₇N₄O₄S (MH⁺), 467.1675; found, 467.1757.

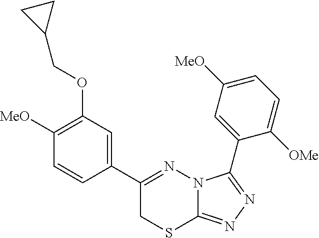

6-(3-(cyclopropylmethoxy)-4-methoxyphenyl)-3-(2,5-dimethoxyphenyl)-7H-[1.2.4]triazolo[3,4-b][1.3.4]thiadiazine (7): pale yellow oil; ¹H NMR (CDCl₃, 400 MHz) 7.46 (d, J=1.96 Hz, 1H), 7.35 (dd, J=2.15, 8.24 Hz, 1H), 7.22 (d, J=3.13 Hz, 1H), 7.07-7.04 (m, 1H), 6.96-6.90 (m, 2H) 3.97 (s, 2H), 3.94 (s, 3H), 3.83 (d, J=6.65 Hz, 2H), 3.81 (s, 3H), 3.70 (s, 3H), 1.32-1.26 (m, 1H), 0.66-0.61 (m, 2H), 0.35-0.31 (m, 2H); ¹³C NMR (CDCl₃, 150 MHz) δ 153.4, 153.1, 152.6, 152.2, 151.3, 148.8, 141.5, 125.7, 121.2, 118.0, 116.4, 115.3, 112.7, 111.4, 110.9, 74.1, 56.3, 56.0, 55.9, 23.1, 10.1, 3.47; LC-MS: RT (min)=5.63; [M+H]⁺ 453.1; HRMS calcd for C₂₃H₂₅N₄O₄S (M+H) 453.1518, found 453.1595.

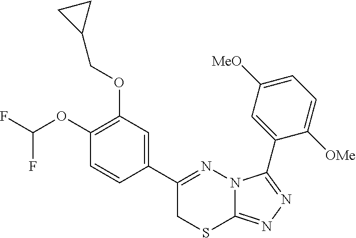

6-(3-(cyclopropylmethoxy)-4-(difluoromethoxy)phenyl)-3-(2,5-dimethoxyphenyl)-7H-[1,2,4]triazolo[3,4-b][1,3,4]thiadiazine (8). yellow oil. ¹H NMR (CDCl₃, 400 MHz) δ 7.46 (d, 1H, J=2.0 Hz), 7.33 (dd, 1H, J=2.4, 8.6 Hz), 7.24 (d, 1H, J=8.2 Hz), 7.20 (d, 1H, J=3.1 Hz), 7.06 (dd, 1H, J=3.1, 9.0 Hz), 6.94 (d, 1H, J=9.0 Hz), 6.70 (t, 1H, J=74.7 Hz), 4.00 (s, 2H), 3.87 (d, 2H, J=7.0 Hz, 2H), 3.81 (s, 3H), 3.70 (s, 3H), 1.22-1.30 (m, 1H), 0.62-0.68 (m, 2H), 0.31-0.36 (m, 2H). ¹³C NMR (CDCl₃, 100 MHz) δ 153.7, 152.6, 152.4, 151.8, 151.1, 143.6, 141.8, 131.9, 122.6, 120.7, 118.4, 116.7, 115.9, 115.3, 113.1, 113.0, 74.4, 56.6, 56.2, 23.7, 10.2, 3.5. LC/MS: RT (min)=6.10; (MH⁺) 489.1. HRMS: (CI+, m/z), calcd for C₂₃H₂₃F₂N₄O₄S (MH⁺), 489.1330; found, 489.1400.

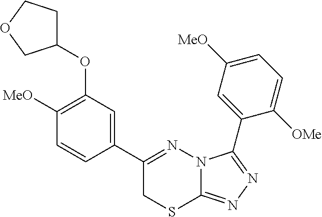

3-(2,5-dimethoxyphenyl)-6-(4-methoxy-3-(tetrahydrofuran-3-yloxy)phenyl)-7H-[1,2,4]triazolo[3,4-b][1,3,4]thiadiazine (9). yellow oil. ¹H NMR (CDCl₃, 400 MHz) δ 7.41 (d, 1H, J=2.0 Hz), 7.38 (dd, 1H, J=2.4, 8.6 Hz), 7.22 (d, 1H, J=3.1 Hz), 7.06 (dd, 1H, J=3.1, 9.0 Hz) 6.94 (dd, 2H, J=8.8, 11.2 Hz), 4.90 (m, 1H), 3.86-4.04 (m, 4H), 3.98 (s, 2H), 3.92 (s, 3H), 3.81 (s, 3H), 3.70 (s, 3H), 2.11-2.16 (m, 2H). ¹³C NMR (CDCl₃, 100 MHz) δ 154.0, 153.6, 152.8, 152.4, 151.4, 147.6, 141.9, 125.7, 122.2, 118.1, 116.9, 115.3, 113.1, 113.0, 111.4, 79.1, 73.0, 67.4, 56.6, 56.3, 56.1, 33.2, 23.1. LC/MS: RT (min)=5.05; (MH⁺) 469.1. HRMS: (CI+, m/z), calcd for C₂₃H₂₅N₄O₅S (MH⁺), 469.1467; found, 469.1544.

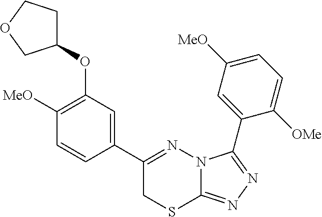

(R)-3-(2,5-dimethoxyphenyl)-6-(4-methoxy-3-(tetrahydrofuran-3-yloxy)phenyl)-7H-[1,2,4]-triazolo[3,4-b][1,3,4]thiadiazine (10). yellow oil. ¹H NMR (CDCl₃, 400 MHz) δ 7.41 (d, 1H, J=2.0 Hz), 7.38 (dd, 1H, J=2.4, 8.6 Hz), 7.22 (d, 1H, J=3.1 Hz), 7.06 (dd, 1H, J=3.1, 9.0 Hz) 6.94 (dd, 2H, J=8.8, 11.2 Hz), 4.90 (m, 1H), 3.86-4.04 (m, 4H), 3.98 (s, 2H), 3.92 (s, 3H), 3.81 (s, 3H), 3.70 (s, 3H), 2.11-2.16 (m, 2H). ¹³C NMR (CDCl₃, 100 MHz) δ 154.0, 153.6, 152.8, 152.4, 151.4, 147.6, 141.9, 125.7, 122.2, 118.1, 116.9, 115.3, 113.1, 113.0, 111.4, 79.1, 73.0, 67.4, 56.6, 56.3, 56.1, 33.2, 23.1. LC/MS: RT (min)=5.05; (MH⁺) 469.1. HRMS: (CI+, m/z), calcd for C₂₃H₂₅N₄O₅S (MH⁺), 469.1540; found, 469.1543.

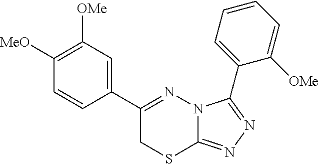

6-(3,4-dimethoxyphenyl)-3-(2-methoxyphenyl)-7H-1,2,41-triazolo[3,4-b][1,3,4]thiadiazine (21). cream solid. Mp 155-156° C. ¹H NMR (d₆-DMSO, 400 MHz) δ 7.55-7.61 (m, 2H), 7.50 (dd, 1H, J=2.0, 8.4 Hz), 7.41 (d, 1H, J=2.0 Hz), 7.24 (d, 1H, J=8.0 Hz), 7.07-7.15 (m, 2H), 4.22 (s, 2H), 3.80 (s, 3H), 3.77 (s, 3H), 3.73 (s, 3H). ¹³C NMR (d₆-DMSO, 100 MHz) δ 158.3, 156.3, 152.9, 150.5, 149.5, 142.9, 6, 132.2, 125.9, 122.4, 121.0, 114.3, 112.6, 112.2, 110.6, 56.5, 56.4, 56.2, 23.5. HRMS: (CI+, m/z), calcd for C₁₉H₁₉N₄O₃S (MH⁺), 383.1100; found, 383.1181.

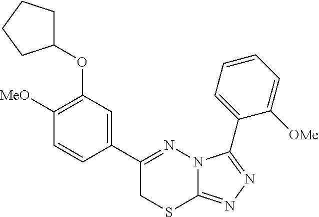

6-(3-(cyclopentyloxy)-4-methoxyphenyl)-3-(2-methoxyphenyl)-7H-[1,2,4]triazolo[3,4-b][1,3,4]thiadiazine (22). yellow oil. ¹H NMR (CDCl₃, 400 MHz) δ 7.63 (dd, 1H, J=1.6, 7.4 Hz), 7.47-7.52 (m, 1H), 7.43 (d, 1H, J=2.0 Hz), 7.30 (dd, 1H, J=2.2, 8.4 Hz), 7.06-7.10 (m, 1H), 7.01 (d, 1H, J=8.2 Hz), 6.89 (d, 1H, J=8.6 Hz), 4.65-4.70 (m, 1H), 3.97 (s, 2H), 3.89 (s, 3H), 3.76 (s, 3H), 1.80-1.88 (m, 6H), 1.57-1.61 (m, 2H). ¹³C NMR (CDCl₃, 100 MHz) δ 158.0, 153.5, 152.4, 151.5, 148.2, 141.3, 132.1, 131.8, 125.7, 120.9, 120.5, 115.1, 112.5, 111.2, 110.9, 80.7, 56.1, 55.7, 32.7, 24.1, 23.0. LC/MS: RT (min)=5.92; (MH⁺) 437.1. HRMS: (CI+, m/z), calcd for C₂₃H₂₅N₄O₃S (MH⁺), 437.1569; found, 437.1649.

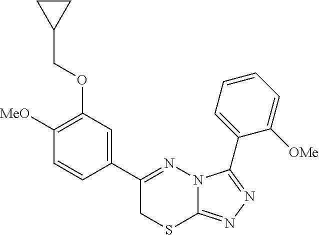

6-(3-(cyclopropylmethoxy)-4-methoxyphenyl)-3-(2-methoxyphenyl)-7H-[1,2,4]triazolo[3,4-b][1,3,4]thiadiazine (23). pale yellow oil. ¹H NMR (CDCl₃, 400 MHz) δ 7.62 (dd, 1H, J=2.0, 7.4 Hz), 7.48-7.52 (m, 1H), 7.41 (d, 1H, J=2.0 Hz), 7.34 (dd, 1H, J=2.2, 8.1 Hz), 7.08 (td, 1H, J=1.0, 7.5 Hz), 7.00 (d, 1H, J=8.2 Hz), 6.91 (d, 1H, J=8.6 Hz), 3.96 (s, 2H), 3.93 (s, 3H), 3.81 (d, 2H, J=7.0 Hz), 3.76 (s, 3H), 1.25-1.29 (m, 1H), 0.60-0.65 (m, 2H), 0.29-0.33 (m, 2H). ¹³C NMR (CDCl₃, 100 MHz) δ 154.4, 149.5, 149.0, 147.9, 145.3, 137.8, 128.6, 128.3, 122.3, 117.7, 117.0, 111.5, 107.9, 107.7, 107.4, 101.2, 70.6, 52.6, 52.2, 19.6, 6.6. LC/MS: RT (min)=5.60; (MH⁺) δ23.1. HRMS: (CI+, m/z), calcd for C₂₂H₂₃N₄O₃S (MH⁺), 423.1413; found, 423.1488.

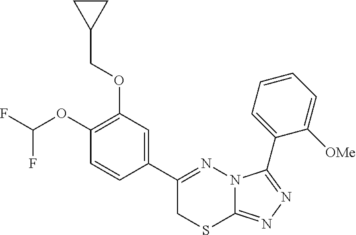

6-(3-(cyclopropylmethoxy)-4-(difluoromethoxy)phenyl)-3-(2-methoxyphenyl)-7H-[1,2,4]triazolo[3,4-b][1,3,4]thiadiazine (24). white solid. Mp 163° C. ¹H NMR (CDCl₃, 400 MHz) δ 7.61 (dd, 1H, J=1.8, 7.6 Hz), 7.48-7.53 (m, 1H), 7.44 (d, 1H, J=2.4 Hz), 7.31-7.34 (m, 1H), 7.22-7.25 (m, 1H), 7.09 (td, 1H, J=1.0, 7.5 Hz), 7.01 (d, 1H, J=8.2 Hz), 6.69 (t, 1H, J=74.7 Hz), 3.97 (s, 2H), 3.85 (d, 2H, J=7.0 Hz), 3.76 (s, 3H), 1.21-1.27 (m, 1H), 0.61-0.67 (m, 2H), 0.31-0.35 (m, 2H). ¹³C NMR (CDCl₃, 100 MHz) δ 157.8, 151.9, 151.5, 150.7, 143.1, 140.9, 132.0, 131.8, 131.7, 122.3, 120.5, 120.3, 115.6, 115.4, 113.0, 112.7, 111.2, 74.1, 55.7, 23.4, 9.9, 3.2. LC/MS: RT (min)=6.07; (MH⁺) δ59.1. HRMS: (CI+, m/z), calcd for C₂₂H₂₁F₂N₄O₃S (MH⁺), 459.1224; found, 459.1304.

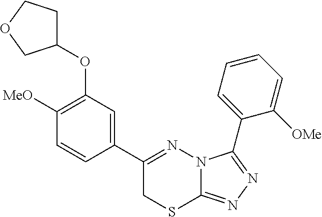

6-(4-methoxy-3-(tetrahydrofuran-3-yloxy)phenyl)-3-(2-methoxyphenyl)-7H-[1,2,4]triazolo[3,4-b][1,3,4]thiadiazine (25). yellow oil. ¹H NMR (CDCl₃, 400 MHz) δ 7.63 (dd, 1H, J=1.6, 7.4 Hz), 7.52-7.57 (m, 1H), 7.38-7.42 (m, 2H), 7.10 (td, 1H, J=1.0, 7.5 Hz), 7.04 (d, 1H, J=7.8 Hz), 6.94 (d, 1H, J=8.2 Hz), 4.87 (tt, 1H, J=2.5, 5.1 Hz), 3.87-4.04 (m, 4H), 4.02 (s, 2H), 3.92 (s, 3H), 3.78 (s, 3H), 2.10-2.16 (m, 2H). ¹³C NMR (CDCl₃, 100 MHz) δ 158.3, 154.2, 153.6, 151.0, 147.6, 142.2, 133.2, 132.0, 125.5, 122.4, 120.8, 113.8, 113.1, 111.6, 111.5, 79.1, 73.0, 67.4, 56.3, 56.0, 33.1, 23.0. LC/MS: RT (min)=4.99; (MH⁺) 439.1. HRMS: (CI+, m/z), calcd for C₂₂H₂₃N₄O₄S (MH⁺), 439.1362; found, 439.1439.

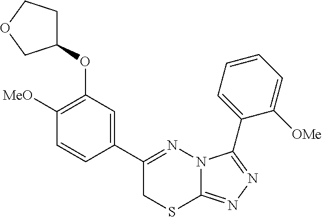

(R)-6-(4-methoxy-3-(tetrahydrofuran-3-yloxy)phenyl)-3-(2-methoxyphenyl)-7H-[1,2,4]-triazolo[3,4-b][1,3,4]thiadiazine (26). yellow oil. ¹H NMR (CDCl₃, 400 MHz) δ 7.62 (dd, 1H, J=1.6, 7.4 Hz), 7.50-7.55 (m, 1H), 7.37-7.40 (m, 2H), 7.09 (td, 1H, J=1.0, 7.5 Hz), 7.03 (d, 1H, J=7.8 Hz), 6.92-6.95 (m, 1H), 4.87 (tt, 1H, J=2.5, 5.1 Hz), 3.86-4.04 (m, 4H), 3.99 (s, 2H), 3.92 (s, 3H), 3.76 (s, 3H), 2.09-2.15 (m, 2H). ¹³C NMR (CDCl₃, 100 MHz) δ 158.2, 154.0, 152.9, 151.4, 147.6, 141.9, 132.8, 132.0, 125.7, 122.2, 120.8, 114.6, 113.1, 111.6, 111.5, 79.1, 73.0, 67.4, 56.3, 56.0, 33.1, 23.1. LC/MS: RT (min)=4.99; (MH⁺) 439.1.

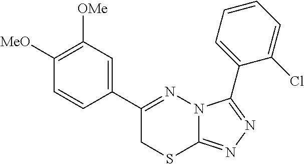

3-(2-chlorophenyl)-6-(3,4-dimethoxyphenyl)-7H-[1,2,4]-triazolo[3,4-b][1,3,4]thiadiazine (27). off-white needles. Mp 225-226° C. ¹H NMR. (CDCl₃, 400 MHz) δ 7.71 (dd, 1H, J=2.0, 7.4 Hz), 7.41-7.54 (m, 4H), 7.34 (dd, 1H, J=2.2, 8.4 Hz), 6.91 (d, 1H, J=8.6 Hz), 3.98 (s, 2H), 3.94 (s, 3H), 3.86 (s, 3H). ¹³C NMR (CDCl₃, 100 MHz) δ 152.9, 152.5, 149.4, 141.6, 132.6, 131.6, 131.5, 129.8, 127.1, 126.8, 125.9, 125.8, 121.2, 110.5, 109.5, 56.0, 55.8, 23.5. LC/MS: RT (min)=5.29; (MH⁺), HRMS: (CI+, m/z), calcd for C₁₈H₁₆ClN₄O₂S (MH), 387.0604; found, 387.0675.

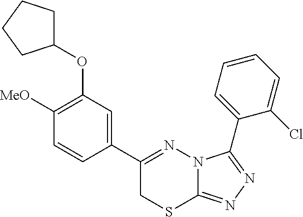

3-(2-chlorophenyl)-6-(3-(cyclopentyloxy)-4-methoxyphenyl)-7H-[1,2,4]triazolo[3,4-b][1,3,4]thiadiazine (28). pale yellow solid. Mp 168° C. ¹H NMR (CDCl₃, 400 MHz) δ 7.69 (dd, 1H, J=2.0, 7.4 Hz), 7.39-7.52 (m, 4H), 7.29 (dd, 1H, J=2.2, 8.4 Hz), 6.88 (d, 1H, J=6.8 Hz), 4.66-4.72 (m, 1H), 3.97 (s, 2H), 3.90 (s, 3H), 1.81-1.89 (m, 6H), 1.59-1.61 (m, 2H). ¹³C NMR (CDCl₃, 100 MHz) δ 153.7, 153.1, 151.9, 148.4, 141.7, 134.6, 132.8, 131.7, 129.9, 127.0, 126.2, 125.8, 121.1, 112.7, 111.1, 80.8, 56.3, 32.8, 24.3, 23.5. LC/MS: RT (min)=6.24; (MH⁺) 441.1. HRMS: (CI+, m/z), calcd for C₂₂H₂₂ClN₄O₂S (MH⁺) 441.1074; found, 425.1455.

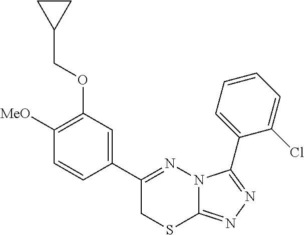

3-(2-chlorophenyl)-6-(3-(cyclopropylmethoxy)-4-methoxyphenyl)-7H-[1,2,4]triazolo[3,4-b][1,3,4]thiadiazine (29). glossy cream needles. Mp 173° C. ¹H NMR (CDCl₃, 400 MHz) δ 7.69 (dd, 1H, J=1.8, 7.2 Hz), 7.40-7.52 (m, 4H), 7.33 (dd, 1H, J=2.2, 8.1 Hz), 6.90 (d, 1H, J=8.6 Hz), 3.97 (s, 2H), 3.93 (s, 3H), 3.83 (d, 2H, J=7.0 Hz), 1.25-1.30 (m, 1H), 0.60-0.65 (m, 2H), 0.30-0.34 (m, 2H). ¹³C NMR (CDCl₃, 100 MHz) δ 149.5, 149.4, 145.2, 130.8, 129.1, 128.1, 126.3, 123.2, 122.3, 122.1, 117.7, 107.9, 107.3, 70.4, 52.5, 27.0, 19.9, 6.5. LC/MS: RT (min)=5.88; (MH⁺) 427.1. HRMS: (CI+, m/z), calcd for C₂₁H₂₀ClN₄O₂S (MH⁺), 427.0917; found, 427.0989.

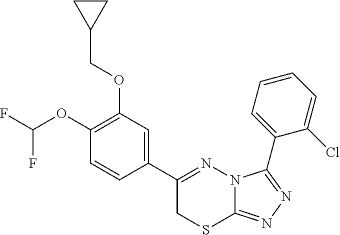

3-(2-chlorophenyl)-6-(3-(cyclopropylmethoxy)-4-(difluoromethoxy)phenyl)-7H-[1,2,4]-triazolo[3,4-b][1,3,4]thiadiazine (30). white needles. Mp 193° C. ¹H NMR (CDCl₃, 400 MHz) δ 7.68-7.72 (m, 1H), 7.41-7.54 (m, 4H), 7.32 (dd, 1H, J=2.0, 8.2 Hz), 7.24 (d, 1H, J=8.2 Hz), 6.70 (t, 1H, J=75.1 Hz), 3.99 (s, 2H), 3.87 (d, 2H, J=7.0 Hz), 1.21-1.32 (m, 1H), 0.62-0.68 (m, 2H), 0.31-0.36 (m, 2H). ¹³C NMR (CDCl₃, 100 MHz) δ 152.7, 152.2, 151.0, 141.8, 134.5, 132.9, 132.0, 131.8, 130.1, 127.2, 126.0, 122.6, 120.7, 118.5, 115.9, 113.1, 74.3, 24.0, 10.2, 3.6. LC/MS: RT (min)=6.32; (MH) δ63.0. HRMS: (CI+, m/z), calcd for C₂₁H₁₈ClF₂N₄O₂S (MH⁺), 463.0729; found, 463.0798.

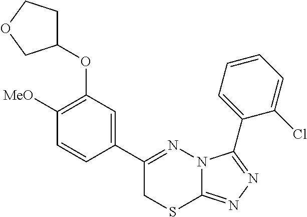

3-(2-chlorophenyl)-6-(4-methoxy-3-(tetrahydrofuran-3-yloxy)phenyl)-7H-[1,2,4]-triazolo[3,4-b][1,3,4]thiadiazine (31). colorless needles. Mp 212° C. (dec.). ¹H NMR (CDCl₃, 400 MHz) δ 7.70 (dd, 1H, J=2.0, 7.4 Hz), 7.47-7.54 (m, 2H), 7.44 (dd, 1H, J=1.8, 7.2 Hz), 7.41 (d, 1H, J=2.0 Hz), 7.35 (dd, 1H, J=2.2, 8.4 Hz), 6.92 (d, 1H, J=8.6 Hz), 4.86-4.90 (m, 1H), 3.87-4.05 (m, 4H), 3.98 (s, 2H), 3.92 (s, 3H), 2.13-2.18 (m, 2H). LC/MS: RT (min)=5.24; (MH⁺) 443.1. HRMS: (CI+, m/z), calcd for C₂₁H₂₀ClN₄O₃S (MH⁺), 443.0866; found, 443.0955.

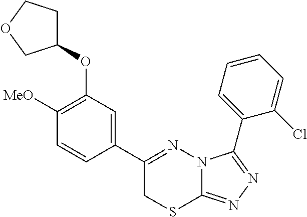

(R)-3-(2-chlorophenyl)-6-(4-methoxy-3-(tetrahydrofuran-3-yloxy)phenyl)-7H-[1,2,4]-triazolo[3,4-b][1,3,4]thiadiazine (32). off-white powder. Mp 218° C. (dec.). ¹H NMR (CDCl₃, 400 MHz) δ 7.70 (dd, 1H, J=1.6, 7.4 Hz), 7.47-7.54 (m, 2H), 7.44 (dd, 111, J=1.6, 7.4 Hz), 7.41 (d, 1H, J=2.0 Hz), 7.35 (dd, 1H, J=2.2, 8.4 Hz), 6.92 (d, 1H, J=8.6 Hz), 4.86-4.90 (m, 1H), 3.87-4.04 (m, 4H), 3.97 (s, 2H), 3.91 (s, 3H), 2.12-2.18 (m, 2H). ¹³C NMR (CDCl₃, 100 MHz) δ 153.8, 152.8, 152.1, 147.7, 141.8, 134.6, 132.9, 132.0, 130.0, 127.1, 126.2, 125.9, 122.0, 112.9, 111.4, 79.0, 73.0, 67.4, 56.3, 33.2, 23.6. LC/MS: RT (min)=5.25; (MH) δ43.1. HRMS: (CI+, m/z), calcd for C₂₁H₂₀ClN₄O₃S (MH⁺), 443.0866; found, 443.0942.

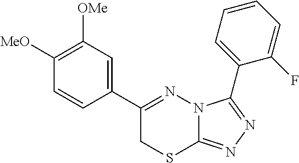

6-(3,4-dimethoxyphenyl)-3-(2-fluorophenyl)-7H-[1,2,4]-triazolo[3,4-b][1,3,4]thiadiazine (33). cream solid. Mp 222° C. (dec.). ¹H NMR (CDCl₃, 400 MHz) δ 7.80 (td, 1H, J=1.8, 7.3 Hz), 7.48-7.54 (m, 1H), 7.45 (d, 1H, J=2.0 Hz), 7.34 (dd, 1H, J=2.2, 8.4), 7.29 (td, 1H, J=1.0, 7.5 Hz), 7.16-7.21 (m, 1H), 6.91 (d, 1H, J=8.2 Hz), 3.98 (s, 2H), 3.93 (s, 3H), 3.86 (s, 3H). ¹³C NMR (CDCl₃, 100 MHz) δ 152.9, 152.5, 149.3, 132.5, 132.4, 131.6, 125.8, 124.3, 124.2, 121.2, 116.0, 115.8, 114.5, 110.5, 109.3, 56.0, 55.8, 23.3. LC/MS: RT (min)=5.15; (MH⁺) 371.1. HRMS: (CI+, m/z), calcd for C₁₈H₁₆FN₄O₂S (MH⁺) 371.0900; found, 371.0979.

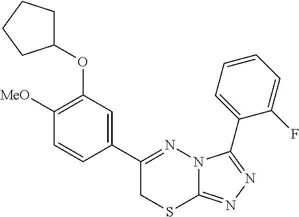

6-(3-(cyclopentyloxy)-4-methoxyphenyl)-3-(2-fluorophenyl)-7H-[1,2,4]-triazolo[3,4-b][1,3,4]thiadiazine (34). yellow oil. ¹H NMR (CDCl₃, 400 MHz) δ 7.79-7.82 (m, 1H), 7.63-7.69 (m, 3H), 7.32 (d, 1H, J=2.0 Hz), 7.24 (dd, 1H, J=2.2, 8.4 Hz), 6.84 (d, 1H, J=8.6 Hz), 4.58-4.63 (m, 1H), 3.94 (s, 2H), 3.86 (s, 3H), 1.75-1.80 (m, 6H), 1.52-1.58 (m, 2H). ¹³C NMR (CDCl₃, 100 MHz) δ 153.5, 153.3, 148.0, 141.2, 132.7, 131.5, 130.7, 130.6, 130.3, 126.8, 126.7, 126.6, 125.3, 122.1, 120.9, 112.4, 110.8, 80.6, 56.0, 32.5, 23.9, 23.2. LC/MS: RT (min)=6.12; (MH⁺) 425.1. HRMS: (CI+, m/z), calcd for C₂₂H₂₂FN₄O₂S (MH⁺), 425.1369; found, 425.1455.

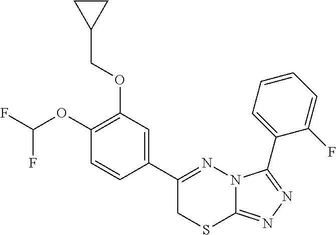

6-(3-(cyclopropylmethoxy)-4-(difluoromethoxy)phenyl)-3-(2-fluorophenyl)-7H-[1,2,4]-triazolo[3,4-b][1,3,4]thiadiazine (35). white powder. Mp 183° C. ¹H NMR (CDCl₃, 400 MHz) δ 7.81 (td, 1H, J=1.8, 7.3 Hz), 7.51-7.57 (m, 1H), 7.50 (d, 1H, J=2.0 Hz), 7.30-7.36 (m, 2H), 7.26 (app. d, 1H, J=8.0 Hz), 7.18-7.23 (m, 1H), 6.70 (t, 1H, J=74.7 Hz), 4.01 (s, 2H), 3.88 (d, 2H, J=7.0 Hz), 1.24-1.33 (m, 1H), 0.63-0.68 (m, 2H), 0.33-0.37 (m, 2H). ¹³C NMR (CDCl₃, 100 MHz) δ 161.6, 159.1, 152.7, 150.3, 142.2, 132.9, 132.8, 131.0, 131.8, 122.7, 120.7, 116.3, 116.1, 115.9, 113.3, 113.0, 74.3, 23.8, 10.2, 3.6. LC/MS: RT (min)=6.21; (MH⁺) 447.1. HRMS: (CI+, m/z), calcd for C₂₁H₁₈F₃N₄O₂S (MH⁺), 447.1024; found, 447.1103.

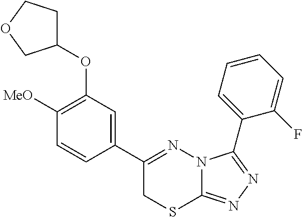

3-(2-fluorophenyl)-6-(4-methoxy-3-(tetrahydrofuran-3-yloxy)phenyl)-7H-[1,2,4]triazolo[3,4-b][1,3,4]thiadiazine (36). white solid. Mp 198-199° C. ¹H NMR (CDCl₃, 400 MHz) δ 7.78 (td, 1H, J=1.8, 7.3 Hz), 7.49-7.55 (m, 1H), 7.43 (d, 1H, J=2.0 Hz, 1H), 7.37 (dd, 1H, J=2.2, 8.4 Hz), 7.31 (td, 1H, J=1.2, 7.6 Hz), 7.19 (ddd, 1H, J=1.0, 8.6, 10.0 Hz), 6.92 (d, 1H, J=8.6 Hz), 4.88-4.92 (m, 1H), 3.85-4.02 (m, 4H), 4.00 (s, 2H), 3.90 (s, 3H), 2.11-2.17 (m, 2H). ¹³C NMR (CDCl₃, 100 MHz) δ 161.6, 159.1, 153.8, 152.9, 150.0, 147.6, 142.2, 132.7, 132.6, 131.9, 129.9, 124.6, 124.5, 122.1, 116.3, 116.1, 114.9, 114.8, 113.1, 111.5, 79.0, 73.0, 67.4, 56.3, 33.1, 23.4. LC/MS: RT (min)=5.12; (MH) δ27.1. HRMS: (CI+, m/z), calcd for C₂₁H₂₀FN₄O₃S (MH⁺), 427.1162; found, 427.1245.

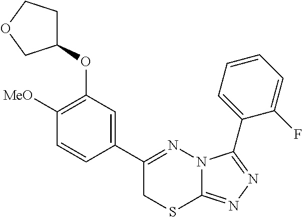

(R)-3-(2-fluorophenyl)-6-(4-methoxy-3-(tetrahydrofuran-3-yloxy)phenyl)-7H-[1,2,4]triazolo[3,4-b][1,3,4]thiadiazine (37). off-white solid. Mp 196-197° C. ¹H NMR (CDCl₃, 400 MHz) δ 7.81 (td, 1H, J=1.8, 7.3 Hz), 7.51-7.57 (m, 1H), 7.44 (d, 1H, J=2.0 Hz), 7.37 (dd, 1H, J=2.2, 8.4 Hz), 7.31 (td, 1H, J=1.2, 7.6 Hz), 7.20 (ddd, 1H, J=1.2, 8.4, 10.0 Hz), 6.93 (d, 1H, J=8.6 Hz), 4.89-4.93 (m, 1H), 3.87-4.04 (m, 4H), 3.99 (s, 2H), 3.91 (s, 3H), 2.13-2.19 (m, 2H). ¹³C NMR (CDCl₃, 100 MHz) δ 161.6, 159.1, 153.8, 152.9, 150.1, 147.6, 142.3, 132.8, 132.7, 132.0, 125.9, 124.7, 124.6, 122.1, 116.3, 116.1, 114.9, 114.8, 112.9, 111.4, 79.02, 73.0, 67.4, 56.3, 33.1, 23.4. LC/MS: RT (min)=5.13; (MH⁺) 427.1. HRMS: (CI+, m/z), calcd for C₂₁H₂₀FN₄O₃S (MH⁺), 427.1162; found, 427.1240.

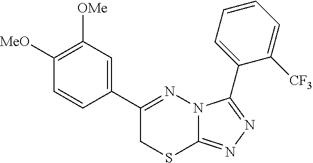

6-(3,4-dimethoxyphenyl)-3-(2-(trifluoromethyl)phenyl)-7H-[1,2,4]-triazolo[3,4-b][1,3,4]thiadiazine (38). white solid. Mp 204-205° C. ¹H NMR (CDCl₃, 400 MHz) δ 7.79-7.82 (m, 1H), 7.65-7.68 (m, 3H), 7.33 (d, 1H, J=2.4 Hz), 7.28 (dd, 1H, J=2.2, 8.4 Hz), 6.87 (d, 1H, J=8.2 Hz), 3.96 (s, 2H), 3.91 (s, 3H), 3.79 (s, 3H). ¹³C NMR (CDCl₃, 100 MHz) δ 153.3, 152.6, 151.4, 149.3, 141.3, 132.8, 131.5, 130.7, 130.2, 126.9, 126.8, 125.7, 122.2, 121.2, 110.5, 109.4, 56.0, 55.8, 23.4. LC/MS: RT (min)=5.43; (MH⁺) δ21.1. HRMS: (CI+, m/z), calcd for C₁₉H₁₆F₃N₄O₂S (MH⁺) 421.0868; found, 421.0948.

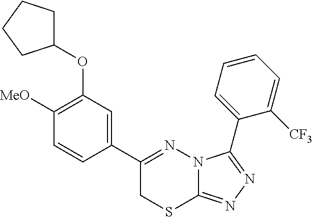

6-(3-(cyclopentyloxy)-4-methoxyphenyl)-3-(2-(trifluoromethyl)phenyl)-7H-[1,2,4]-triazolo[3,4-b][1,3,4]thiadiazine (39). yellow oil. NMR (CDCl₃, 400 MHz) δ7.80 (dd, 1H, J=2.7, 7.0 Hz), 7.64-7.68 (m, 3H), 7.32 (d, 1H, J=2.4 Hz), 7.23 (dd, 1H, J=2.2, 8.4 Hz), 6.84 (d, 1H, J=8.6 Hz), 4.59-4.64 (m, 1H), 3.94 (s, 2H), 3.86 (s, 3H), 1.75-1.80 (m, 6H), 1.53-1.59 (m, 2H). ¹³C NMR (CDCl₃, 100 MHz) δ 153.5, 153.2, 151.3, 148.0, 141.1, 132.7, 131.5, 130.5, 126.8, 126.7, 126.6, 125.4, 120.8, 112.4, 110.8, 80.6, 56.0, 32.5, 23.9, 23.2. LC/MS: RT (min)=6.30; (MH⁺) 475.1. HRMS: (CI+, m/z), calcd for C₂₃H₂₂F₃N₄O₂S (MH), 475.1337; found, 475.1416.

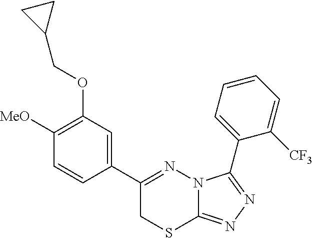

6-(3-(cyclopropylmethoxy)-4-methoxyphenyl)-3-(2-(trifluoromethyl)phenyl)-7H-[1,2,4]-triazolo[3,4-b][1,3,4]thiadiazine (40). white solid. Mp 166° C. (dec.). ¹H NMR (CDCl₃, 400 MHz) δ 7.81-7.84 (m, 1H), 7.66-7.71 (m, 3H), 7.35 (d, 1H, J=2.4 Hz), 7.29 (dd, 1H, J=2.4, 8.6 Hz), 6.88 (d, 1H, J=8.2 Hz), 3.96 (s, 2H), 3.92 (s, 3H), 3.78 (d, 2H, J=7.0 Hz), 1.21-1.29 (m, 1H), 0.58-0.64 (m, 2H), 0.27-0.31 (m, 2H). LC/MS: RT (min)=5.97; (MH⁺) 461.1.

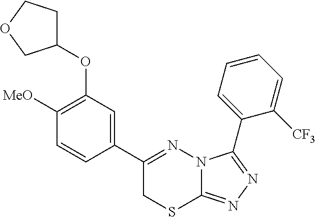

6-(4-methoxy-3-(tetrahydrofuran-3-yloxy)phenyl)-3-(2-(trifluoromethyl)phenyl)-7H-[1,2,4]-triazolo[3,4-b][1,3,4]thiadiazine (41). white solid. Mp 186° C. (dec.). ¹H NMR (CDCl₃, 400 MHz) δ 7.82-7.85 (m, 1H), 7.66-7.73 (m, 3H), 7.33 (d, 1H, J=2.0 Hz), 7.29-7.31 (m, 1H), 6.90 (d, 1H, J=8.2 Hz), 4.82 (tt, 1H, J=2.4, 5.4 Hz), 3.84-4.05 (m, 4H), 3.97 (s, 2H), 3.90 (s, 3H), 2.03-2.12 (m, 2H). LC/MS: RT (min)=5.38; (MH⁺) 477.1. HRMS: (CI+, m/z), calcd for C₂₂H₂₀F₃N₄O₃S (MH⁺), 477.1130; found, 477.1205.

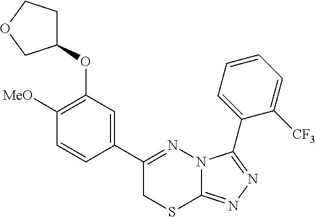

(R)-6-(4-methoxy-3-(tetrahydrofuran-3-yloxy)phenyl)-3-(2-(trifluoromethyl)phenyl)-7H-[1,2,4]triazolo[3,4-b][1,3,4]thiadiazine (42). off-white needles. Mp 199-200° C. ¹H NMR (CDCl₃, 400 MHz) δ 7.82-7.85 (m, 1H), 7.65-7.73 (m, 3H), 7.27-7.33 (m, 2H), 6.92 (d, 1H, J=8.2 Hz), 4.79-4.84 (m, 1H), 3.84-3.99 (m, 4H), 3.96 (s, 2H), 3.90 (s, 3H), 2.04-2.11 (m, 2H). LC/MS: RT (min)=5.38; (MH⁺) 477.1. HRMS: (CI+, m/z), calcd for C₂₂H₂₀F₃N₄O₃S (MH⁺), 477.1130; found, 477.1203.

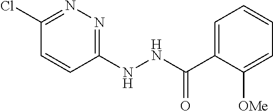

### N′-(6-chloropyridazin-3-yl)-2-methoxybenzohydrazide (43)

Method A: To a stirred solution of o-anisic acid (2.07 g, 13.59 mmol, 1.0 eq) in DMF (54 mL, 0.25M) under N₂ at room temperature was added 1,1′-carbonyldiimidazole (2.43 g, 14.95 mmol, 1.1 eq). After stirring for 30 min, 3-chloro-6-hydrazinopyridazine (1.97 g, 13.59 mmol, 1.0 eq) was added and the solution was stirred at room temperature for an additional 1 h. The reaction mixture was poured into H₂O and the resultant precipitate was filtered, washed with H₂O then hexane, and dried under reduced pressure to provide hydrazide 1 (2.08 g, 55%) as a white solid.

Method B: To a stirred solution of 3-chloro-6-hydrazinylpyridazine (1.03 g, 7.14 mmol, 1.0 eq) in Et₂O (29 mL, 0.25M) under N₂ at room temperature was added triethylamine (1.0 mL, 0.72 g, 7.14 mmol, 1.0 eq) followed by 2-methoxybenzoyl chloride (1.1 mL, 1.22 g, 7.14 mmol, 1.0 eq) dropwise slowly. After stirring at room temperature for 1 h, the precipitate was filtered, washed with H₂O then hexane, and dried under reduced pressure to provide hydrazide 43 (1.99 g, quant.) as a white solid. R_{f}=0.49 (CH₂Cl₂/MeOH 95:5). Mp 211° C. (dec.). IR (neat, diamond/ZnSe) 3313, 3204, 3113, 3070, 3026, 1659, 1637, 1592, 1523, 1484, 1470, 1460, 1431, 1292, 1243, 1182, 1166, 1148, 1109, 1078, 1040, 1008, 951, 906, 851, 832, 798, 786, 753, 693, 667 cm⁻¹. ¹H NMR (400 MHz, d₆-DMSO) δ 10.18 (d, 1H, J=1.3 Hz, NH), 9.41 (d, 1H, J=1.3 Hz, NH), 7.70 (dd, 1H, J=1.8, 7.6 Hz, aryl), 7.59 (d, 1H, J=9.3 Hz, aryl), 7.52 (ddd, 1H, J=1.8, 7.5, and 8.2 Hz, aryl), 7.18 (d, 1H, J=8.3 Hz, aryl), 7.08 (d, 1H, J=9.4 Hz, aryl), 7.07 (dt, 1H, J=0.6, 7.5 Hz, aryl), 3.92 (s, 3H, Me). ¹³C NMR (100 MHz, d₆-DMSO) δ 165.3, 160.2, 157.0, 147.4, 132.6, 130.1, 129.5, 121.9, 120.5, 116.2, 112.0, 55.9. LC/MS: RT (min)=4.02; (MH⁺) 279.1. HRMS: (CI+, m/z), calcd for C₁₂H₁₂ClN₄O₂ (MH⁺), 279.0649; found, 279.0648.

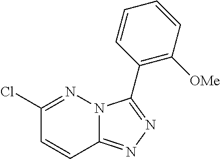

### 6-chloro-3-(2-methoxyphenyl)-[1,2,4]-triazolo[4,3-b]pyridazine (44)

Method A: To a stirred suspension of N′-(4-chlorophenyl)-2-methoxybenzohydrazide (43) (1.02 g, 3.65 mmol, 1.0 eq) in o-xylene under N₂ at room temperature was added triethylamine hydrochloride (251 mg, 1.83 mmol, 0.5 eq). After refluxing for 16 h, the reaction mixture was cooled to room temperature and concentrated under reduced pressure to give a residue. The crude material was diluted with CH₂Cl₂, washed with brine (2×), dried over MgSO₄, and filtered. Removal of the solvent under reduced pressure gave a crude solid which was recrystallized from Et₂O to give [1,2,4]triazolo[4,3-b]pyridazine 44 (115 mg, 12%) as a white solid.

Method B: A solution of N′-(4-chlorophenyl)-2-methoxybenzohydrazide (1) (524 mg, 1.88 mmol, 1.0 eq) in phosphorus oxychloride (9.4 mL, 0.2M) under N₂ was heated at 105° C. for 2 h. The reaction mixture was cooled to room temperature and concentrated under reduced pressure to give a residue. The crude material was diluted with CH₂Cl₂ and sat. aq. NaHCO₃ was added dropwise until pH 8 was obtained. The biphasic solution was separated and the aqueous layer was extracted with CH₂Cl₂ (1×). The organic layers were combined, washed with brine (1×), dried over MgSO₄, and filtered. Removal of the solvent under reduced pressure gave an oil which was recrystallized from Et₂O to give [1,2,4]triazolo[4,3-b]pyridazine 44 (458 mg, 94%) as a white solid. R_{f}=0.60 (CH₂Cl₂/MeOH 95:5). Mp 140-141° C. IR (neat, diamond/ZnSe) 3081, 3048, 3019, 2934, 2836, 1609, 1585, 1532, 1519, 1480, 1461, 1444, 1431, 1383, 1351, 1327, 1277, 1257, 1181, 1159, 1149, 1124, 1101, 1050, 1037, 1028, 984, 938, 827, 800, 779, 741, 710, 666 cm⁻¹. ¹H NMR (400 MHz, d₆-DMSO) δ 8.52 (d, 1H, J=9.7 Hz, aryl), 7.62 (ddd, 1H, J=1.8, 7.5, and 8.5 Hz, aryl), 7.54 (dd, 1H, J=1.7, 7.5 Hz, aryl), 7.54 (d, 1H, J=9.6 Hz, aryl), 7.28 (d, 1H, J=8.0 Hz, aryl), 7.16 (dt, 1H, J=0.9, 7.5 Hz, aryl), 3.77 (s, 3H, Me). ¹³C NMR (100 MHz, d₆-DMSO) δ 158.0, 148.9, 146.6, 143.1, 132.6, 131.7, 127.0, 122.9, 120.6, 114.3, 112.3, 55.8. LC/MS: RT (min)=4.58; (MH⁺) 261.0. HRMS: (CI+, m/z), calcd for C₁₂H₁₀ClN₄O (MH⁺), 261.0543; found, 261.0551.

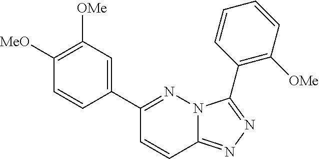

6-(3,4-dimethoxyphenyl)-3-(2-methoxyphenyl)-[1,2,4]triazolo[4,3-b]pyridazine (45). To a suspension of 6-chloro-3-(2-methoxyphenyl)-[1,2,4]triazolo[4,3-b]pyridazine (44) (50 mg, 0.19 mmol, 1.0 eq) in DME (1.9 mL, 0.1M) in a microwave tube was added 3,4-dimethoxyphenylboronic acid (105 mg, 0.57 mmol, 3.0 eq), Pd(PPh₃)₄ (11 mg, 9.57 μmol, 5 mol %), and 2.0M aq. Na₂CO₃ soln. (0.19 mL, 0.38 mmol, 2.0 eq). The solution was sparged with Ar for 5 min and then heated at 150° C. in a microwave for 30 min. After cooling to room temperature, the reaction mixture was diluted with EtOAc and filtered through a silica gel plug. The filtrate was washed with brine (1×), dried over MgSO₄, and filtered. Removal of the solvent under reduced pressure gave a residue, which was purified by semi-preparative HPLC to give [1,2,4]triazolo[4,3-b]pyridazine 45 (31 mg, 44%) as a white solid. R_{f}=0.46 (CH₂Cl₂/MeOH 95:5). Mp 95-97° C. IR (neat, diamond/ZnSe) 3100, 2941, 2847, 1740, 1610, 1597, 1585, 1514, 1493, 1465, 1440, 1417, 1358, 1340, 1284, 1257, 1225, 1194, 1176, 1155, 1130, 1097, 1065, 1017, 997, 895, 880, 814, 774, 760, 714 cm¹. ¹H NMR (400 MHz, d₆-DMSO) δ 8.46 (d, 1H, J=9.8 Hz, aryl), 8.03 (d, 1H, J=9.8 Hz, aryl), 7.60-7.65 (m, 3H, aryl), 7.55 (d, 1H, J=2.1 Hz, aryl), 7.31 (dd, 1H, J=0.9, 9.0 Hz, aryl), 7.17 (dt, 1H, J=0.9, 7.5 Hz, aryl), 7.11 (d, 1H, J=8.6 Hz, aryl), 3.82 (s, 3H, Me), 3.82 (s, 3H, Me), 3.81 (s, 3H, Me). ¹³C NMR (100 MHz, d₆-DMSO) δ 157.9, 152.6, 151.3, 149.1, 147.1, 143.5, 132.3, 131.8, 126.4, 124.7, 120.6, 120.5, 120.1, 115.0, 111.9 (2C), 109.9, 55.8, 55.7, 55.5. LC/MS: RT (min)=5.03; (MH⁺) 363.2. HRMS: (CI+, m/z), calcd for C₂₀H₁₉N₄O₃ (MH), 363.1457; found, 363.1463.

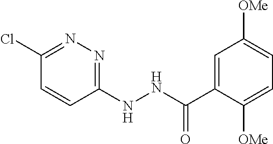

### N′-(6-chloropyridazin-3-yl)-2,5-dimethoxybenzohydrazide (13)

Method A: To a stirred solution of 2,5-dimethoxybenzoic acid (11) (2.01 g, 11.02 mmol, 1.0 eq) in DMF (44.0 mL, 0.25M) under N₂ at room temperature was added 1,1′-carbonyldiimidazole (1.97 g, 14.95 mmol, 1.1 eq). After stirring for 30 min, 3-chloro-6-hydrazinopyridazine (12) (1.97 g, 12.12 mmol, 1.1 eq) was added and the solution was stirred at room temperature for an additional 1 h. The reaction mixture was poured into H₂O and the resultant precipitate was filtered, washed with H₂O then hexane, and dried under reduced pressure to provide hydrazide 13 (1.78 g, 52%) as a white solid.

Method B: To a stirred solution of 2,5-dimethoxybenzoic acid (11) (2.10 g, 11.51 mmol, 1.0 eq) in Et₂O (46.0 mL, 0.25M) under N₂ at 0° C. was added DMF (45 μL, 42 mg, 0.58 mmol, 5 mol %) followed by oxalyl chloride (5.0 mL, 7.30 g, 57.50 mmol, 5.0 eq) slowly dropwise then warmed to room temperature and stirred for 1 h. The solution was concentrated under reduced pressure to give a viscous oil which was added slowly dropwise to a stirred solution of 3-chloro-6-hydrazinylpyridazine (12) (1.66 g, 11.51 mmol, 1.0 eq) and triethylamine (1.60 mL, 1.17 g, 11.51 mmol, 1.0 eq) in Et₂O (46.0 mL, 0.25M) under N₂ at rt. After stirring at room temperature for 1 h, the precipitate was filtered, washed with H₂O then hexane, and dried under reduced pressure to provide hydrazide 13 (3.40 g, 96%) as a white solid. R_{f}=0.41 (CH₂Cl₂/MeOH 95:5); 0.58 (EtOAc). Mp 189° C. (dec.). IR (neat, diamond/ZnSe) 3309, 3210, 3185, 3069, 3039, 3002, 2966, 2943, 2837, 1665, 1641, 1595, 1579, 1526, 1492, 1453, 1408, 1313, 1283, 1261, 1215, 1176, 1160, 1135, 1081, 1064, 1040, 1020, 958, 931, 891, 875, 839, 805, 782, 765, 733, 712 cm⁻¹. ¹H NMR (400 MHz, d₆-DMSO) δ 10.21 (s, 1H, NH), 9.43 (s, 1H, NH), 7.58 (d, 1H, J=9.0 Hz, aryl), 7.26 (d, 1H, J=2.7 Hz, aryl), 7.07-7.14 (m, 3H, aryl), 3.88 (s, 3H, Me), 3.75 (s, 3H, Me). ¹³C NMR (100 MHz, d₆-DMSO) δ 164.7, 160.0, 153.0, 151.1, 147.4, 129.5, 122.2, 117.9, 116.3, 114.9, 113.5, 56.4, 55.6. LC/MS: RT (min)=4.20; (MH⁺) δ09.1. HRMS: (CI+, m/z), calcd for C₁₃H₁₄ClN₄O₃ (MH⁺), 309.0754; found, 309.0754.

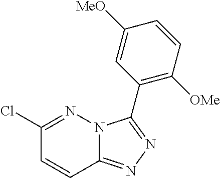

6-chloro-3-(2,5-dimethoxyphenyl)-[1,2,4]-triazolo[4,3-b]pyridazine (14). A solution of N′-(6-chloropyridazin-3-yl)-2,5-dimethoxybenzohydrazide (13) (569 mg, 1.84 mmol, 1.0 eq) in phosphorus oxychloride (9.2 mL, 0.2M) under N₂ was heated at 105° C. for 2 h. The reaction mixture was cooled to room temperature and concentrated under reduced pressure to give a residue. The crude material was diluted with CH₂Cl₂ and sat. aq. NaHCO₃ was added dropwise until pH 8 was obtained. The biphasic solution was separated and the aqueous layer was extracted with CH₂Cl₂ (1×). The organic layers were combined, washed with brine (1×), dried over MgSO₄, and filtered. Removal of the solvent under reduced pressure gave an oil, which was purified by column chromatography on silica gel using CH₂Cl₂/MeOH (95:5) as the eluent to give [1,2,4]triazolo[4,3-b]pyridazine 14 (457 mg, 85%) as a white solid. R_{f}=0.43 (CH₂Cl₂/MeOH 95:5); 0.33 (EtOAc). Mp 111-112° C. IR (neat, diamond/ZnSe) 3093, 2943, 2845, 1757, 1628, 1591, 1524, 1489, 1471, 1438, 1343, 1291, 1275, 1187, 1130, 1069, 1050, 1025, 971, 876, 862, 810, 782, 759, 735, 719, 709 cm⁻¹. ¹H NMR (400 MHz, d₆-DMSO) δ 8.52 (d, 1H, J=9.4 Hz, aryl), 7.53 (d, 1H, J=9.8 Hz, aryl), 7.17-7.23 (m, 211, aryl), 7.12 (d, 1H, J=2.4 Hz, aryl), 3.77 (s, 3H, Me), 3.72 (s, 3H, Me). ¹³C NMR (100 MHz, d₆-DMSO) δ 153.0, 152.1, 148.9, 146.5, 143.1, 127.0, 122.8, 117.6, 116.8, 115.0, 113.7, 56.3, 55.7. LC/MS: RT (min)=4.69; (MH⁺) 291.0. HRMS: (CI+, m/z), calcd for C₁₃H₁₂ClN₄O₂ (MH⁺), 291.0649; found, 291.0649.

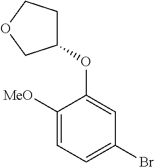

(S)-(+)-3-(5-bromo-2-methoxyphenoxy)tetrahydrofuran (46). To a stirred solution of 5-bromo-2-methoxyphenol (2.51 g, 12.36 mmol, 1.0 eq) in THF (124 mL, 0.1M) under N₂ at room temperature was sequentially added (R)-(−)-tetrahydrofuran-3-ol (1.19 mL, 1.30 g, 14.83 mmol, 1.2 eq), PPh₃ (5.19 g, 19.78 mmol, 1.6 eq), and DEAD (40 wt % in toluene) (9.0 mL, 8.61 g, 19.78 mmol, 1.6 eq). After stirring at room temperature for 16 h, the reaction mixture was concentrated under reduced pressure and purified by column chromatography on silica gel using hexanes/EtOAc (3:1) as the eluent to give (S)-(2-methoxyphenoxy)THF 46 (2.59 g, 78%) as a white solid. R_{f}=0.36 (hexane/EtOAc 3:1); R_{f}=0.56 (hexane/EtOAc 1:1). Mp 66-68° C. [α]_{D}²³ 9.8 (c 2.44, MeOH). IR (neat, diamond/ZnSe) 3017, 2977, 2949, 2915, 2855, 1587, 1498, 1467, 1436, 1399, 1349, 1322, 1251, 1218, 1182, 1132, 1094, 1065, 1021, 991, 968, 911, 892, 844, 796 cm⁻¹. ¹H NMR (400 MHz, d₆-DMSO) δ 7.07-7.09 (m, 2H, aryl), 6.93 (d, 1H, J=8.2 Hz, aryl), 5.02 (m, 1H), 3.71-3.86 (m, 4H), 3.74 (s, 3H, Me), 2.13-2.22 (m, 1H), 1.91-1.99 (m, 1H). ¹³C NMR (100 MHz, d₆-DMSO) δ 149.1, 147.5, 123.7, 117.3, 113.9, 111.6, 78.3, 72.1, 66.4, 55.7, 32.3. LC/MS: RT (min)=5.51; (MH⁺) 273.0. HRMS: (CI+, m/z), calcd for C₁₁H₁₄BrO₃ (MH⁺), 273.0126; found, 273.0127.

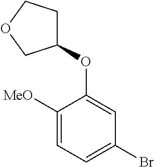

(R)-(−)-3-(5-bromo-2-methoxyphenoxy)tetrahydrofuran (47). To a stirred solution of 5-bromo-2-methoxyphenol (2.50 g, 12.33 mmol, 1.0 eq) in THF (123 mL, 0.1M) under N₂ at room temperature was sequentially added (S)-(+)-tetrahydrofuran-3-ol (1.19 mL, 1.30 g, 14.80 mmol, 1.2 eq), PPh₃ (5.18 g, 19.73 mmol, 1.6 eq), and DEAD (40 wt % in toluene) (9.0 mL, 8.59 g, 19.73 mmol, 1.6 eq). After stirring at room temperature for 16 h, the reaction mixture was concentrated under reduced pressure and purified by column chromatography on silica gel using hexanes/EtOAc (3:1) as the eluent to give (R)-(2-methoxyphenoxy)THF 47 (2.61 g, 78%) as a white solid. R_{f}=0.36 (hexane/EtOAc 3:1); R_{f}=0.56 (hexane/EtOAc 1:1). Mp 66-68° C. [α]_{D}²³ −10.3 (c 2.42, MeOH). IR (neat, diamond/ZnSe) 3017, 2977, 2949, 2915, 2855, 1587, 1498, 1468, 1435, 1399, 1349, 1323, 1252, 1218, 1182, 1132, 1095, 1065, 1021, 992, 968, 912, 892, 844, 796 cm⁻¹. ¹H NMR (400 MHz, d₆-DMSO) δ 7.07-7.09 (m, 2H, aryl), 6.93 (d, 1H, J=8.2 Hz, aryl), 5.02 (m, 1H), 3.69-3.86 (m, 4H), 3.74 (s, 3H, Me), 2.13-2.22 (m, 1H), 1.91-1.99 (m, 1H). ¹³C NMR (100 MHz, d₆-DMSO) δ 149.1, 147.5, 123.7, 117.3, 113.9, 111.6, 78.3, 72.1, 66.4, 55.7, 32.3. LC/MS: RT (min)=5.51; (MH⁺) 273.0. HRMS: (CI+, m/z), calcd for C₁₁H₁₄BrO₃ (MH⁺), 273.0126; found, 273.0127.

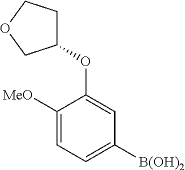

(S)-(+)-4-methoxy-3-(tetrahydrofuran-3-yloxy)phenylboronic acid (15). To a stirred of (S)-(+)-3-(5-bromo-2-methoxyphenoxy)tetrahydrofuran (46) (405 mg, 1.48 mmol, 1.0 eq) in THF (7.4 mL, 0.2M) under N₂ at −78° C. was added n-butyllithium (1.6M in hexane) (1.0 mL, 1.63 mmol, 1.1 eq) dropwise. After stirring at −78° C. for 1 h, trimethylborate (0.25 mL, 231 mg, 2.23 mmol, 1.5 eq) was added dropwise to the solution which was stirred an additional 1 h at −78° C. then warmed to rt. After stirring at room temperature for 16 h, the reaction mixture was quenched with sat. aq. NH₄Cl and concentrated under reduced pressure. The residue was adjusted to pH 3 by addition of aq. 10% HCl soln. and extracted with CH₂Cl₂ (3×). The combined organic layers were diluted with brine and the biphasic solution was stirred at room temperature for 20 min. Subsequently, the organic layer was separated, dried over MgSO₄, and filtered. Removal of the solvent under reduced pressure gave a pasty, yellowish-white solid, which was purified by column chromatography on silica gel using CH₂Cl₂/MeOH (95:5) as the eluent to give the (S)-phenylboronic acid 15 (315 mg, 89%) as a white solid. R_{f}=0.40 (CH₂Cl₂/MeOH 95:5). Mp 198-200° C. [α]_{D}²³ 8.0 (c 1.18, MeOH). IR (neat, diamond/ZnSe) 3360, 2954, 2941, 2866, 2837, 1595, 1517, 1412, 1348, 1319, 1252, 1213, 1179, 1136, 1110, 1077, 1019, 970, 909, 878, 814, 774, 743, 714, 674 cm¹. ¹H NMR (400 MHz, d₆-DMSO) δ 7.47 (dd, 1H, J=1.2, 7.8 Hz, aryl), 7.35 (d, 1H, J=1.2 Hz, aryl), 7.00 (d, 1H, J=8.2 Hz, aryl), 5.01 (m, 1H), 3.73-3.90 (m, 4H), 3.78 (s, 3H, Me), 2.12-2.21 (m, 1H), 1.99-2.05 (m, 1H). ¹³C NMR (100 MHz, d₆-DMSO) δ 151.2, 145.7, 127.5, 120.0, 111.8, 78.1, 72.4, 66.4, 55.4, 32.6. LC/MS: RT (min)=3.53; (MH⁺) 239.1. HRMS: (CI+, m/z), calcd for C₁₁H₁₆BO₅ (MH⁺), 239.1091; found, 239.1092.

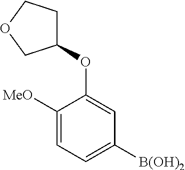

(R)-(−)-4-methoxy-3-(tetrahydrofuran-3-yloxy)phenylboronic acid (16). To a stirred of (R)-(−)-3-(5-bromo-2-methoxyphenoxy)tetrahydrofuran (47) (405 mg, 1.48 mmol, 1.0 eq) in THF (7.4 mL, 0.2M) under N₂ at −78° C. was added n-butyllithium (1.6M in hexane) (1.0 mL, 1.63 mmol, 1.1 eq) dropwise. After stirring at −78° C. for 1 h, trimethylborate (0.25 mL, 231 mg, 2.22 mmol, 1.5 eq) was added dropwise to the solution which was stirred an additional 1 h at −78° C. then warmed to rt. After stirring at room temperature for 16 h, the reaction mixture was quenched with sat. aq. NH₄Cl and concentrated under reduced pressure. The residue was adjusted to pH 3 by addition of aq. 10% HCl soln. and extracted with CH₂Cl₂ (3×). The combined organic layers were diluted with brine and the biphasic solution was stirred at room temperature for 20 min. Subsequently, the organic layer was separated, dried over MgSO₄, and filtered. Removal of the solvent under reduced pressure gave a pasty, yellowish-white solid, which was purified by column chromatography on silica gel using CH₂Cl₂/MeOH (95:5) as the eluent to give the (R)-phenylboronic acid 16 (309 mg, 87%) as a white solid. R_{f}=0.40 (CH₂Cl₂/MeOH 95:5). Mp 198-200° C. [α]_{D}²³ −8.6 (c 1.16, MeOH). IR (neat, diamond/ZnSe) 3358, 2954, 2941, 2865, 2838, 1595, 1517, 1413, 1348, 1319, 1251, 1213, 1179, 1136, 1110, 1077, 1019, 970, 909, 878, 814, 774, 743, 714, 674 cm⁻¹. ¹H NMR (400 MHz, d₆-DMSO) δ 7.47 (dd, 1H, J=1.2, 7.8 Hz, aryl), 7.35 (d, 1H, J=1.2 Hz, aryl), 7.00 (d, 1H, J=8.2 Hz, aryl), 5.01 (m, 1H), 3.73-3.90 (m, 4H), 3.78 (s, 3H, Me), 2.11-2.21 (m, 1H), 1.99-2.05 (m, 1H). ¹³C NMR (100 MHz, d₆-DMSO) δ 151.1, 145.7, 127.4, 120.0, 111.8, 78.1, 72.4, 66.4, 55.4, 32.6. LC/MS: RT (min)=3.54; (MH⁺) 239.1. HRMS: (CI+, m/z), calcd for C₁₁H₁₆BO₅ (MH⁺), 239.1091; found, 239.1091.

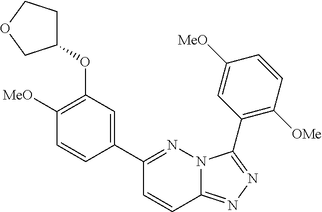

(S)-(+)-3-(2,5-dimethoxyphenyl)-6-(4-methoxy-3-(tetrahydrofuran-3-yloxy)phenyl)-[1,2,4]-triazolo[4,3-b]pyridazine (17). To a suspension of 6-chloro-3-(2,5-dimethoxyphenyl)-[1,2,4]triazolo[4,3-b]pyridazine (14) (115 mg, 0.39 mmol, 1.0 eq) in DME (3.9 mL, 0.1M) in a microwave tube was added (S)-(+)-4-methoxy-3-(tetrahydrofuran-3-yloxy)phenylboronic acid (15) (282 mg, 1.18 mmol, 3.0 eq), Pd(PPh₃)₄ (23 mg, 20.00 mmol, 5 mol %), and 2.0M aq. Na₂CO₃ soln. (0.39 mL, 0.79 mmol, 2.0 eq). The solution was sparged with Ar for 5 min and then heated at 90° C. in a microwave for 30 min. After cooling to room temperature, the reaction mixture was diluted with EtOAc and filtered through a silica gel plug. The filtrate was washed with brine (1×), dried over MgSO₄, and filtered. Removal of the solvent under reduced pressure gave a residue, which was purified by semi-preparative HPLC to give [1,2,4]triazolo[4,3-b]pyridazine 17 (63 mg, 35%) as a white solid. R_{f}=0.40 (CH₂Cl₂/MeOH 95:5); 0.06 (EtOAc). Mp 120-121° C. [α]_{D}²³ 17.3 (c 1.04, CH₂Cl₂). IR (neat, diamond/ZnSe) 3083, 2939, 2838, 1601, 1586, 1514, 1485, 1465, 1427, 1383, 1356, 1329, 1303, 1276, 1256, 1217, 1179, 1153, 1112, 1070, 1042, 1019, 1001, 976, 902, 870, 804, 779, 758, 745, 706, 678, 659 cm⁻¹. ¹H NMR (400 MHz, d₆-DMSO) δ 8.45 (d, 1H, J=9.8 Hz, aryl), 8.01 (d, 1H, J=9.8 Hz, aryl), 7.66 (dd, 1H, J=2.2, 8.4 Hz, aryl), 7.53 (d, 1H, J=2.0 Hz, aryl), 7.18-7.25 (m, 3H, aryl), 7.15 (d, 1H, J=8.6 Hz, aryl), 5.02 (m, 1H), 3.75-3.88 (m, 4H), 3.83 (s, 3H, Me), 3.78 (s, 3H, Me), 3.74 (s, 3H, Me), 2.11-2.20 (m, 1H), 1.95-2.01 (m, 1H). ¹³C NMR (100 MHz, d₆-DMSO) δ 152.9, 152.2, 152.0 (2C), 146.7 (2C), 143.5, 126.4, 124.6, 121.1, 119.8, 117.1, 116.9, 115.7, 113.2, 112.9, 112.4, 78.2, 72.2, 66.4, 56.2, 55.7, 55.6, 32.4. LC/MS: RT (min)=4.99; (MH⁺) 449.1. HRMS: (CI+, m/z), calcd for C₂₄H₂₅N₄O₅ (MH⁺), 449.1825; found, 449.1829.

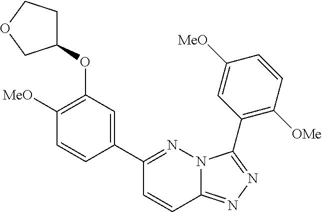

(R)-(−)-3-(2,5-dimethoxyphenyl)-6-(4-methoxy-3-(tetrahydrofuran-3-yloxy)phenyl)-[1,2,4]-triazolo[4,3-b]pyridazine (18). To a suspension of 6-chloro-3-(2,5-dimethoxyphenyl)-[1,2,4]triazolo[4,3-b]pyridazine (14) (529 mg, 1.82 mmol, 1.0 eq) in THF (9.1 mL, 0.2M) in a microwave tube was added (R)-(−)-4-methoxy-3-(tetrahydrofuran-3-yloxy)phenylboronic acid (16) (1.30 g, 5.46 mmol, 3.0 eq), Pd(OAc)₂ (20 mg, 91.0 mmol, 5 mol %), and KF (317 mg, 5.46 mmol, 3.0 eq). The solution was sparged with Ar for 5 min and then heated at 90° C. in a microwave for 45 min. After cooling to room temperature, the reaction mixture was filtered through an SPE column which was then flushed with MeOH. The combined filtrate was concentrated under reduced pressure to give a residue, which was purified by semi-preparative HPLC to give [1,2,4]triazolo[4,3-b]pyridazine 18 (692 mg, 85%) as a white solid. R_{f}=0.40 (CH₂Cl₂/MeOH 95:5); 0.06 (EtOAc). Mp 120-121° C. [α]_{D}²³ −19.2 (c 1.04, CH₂Cl₂). IR (neat, diamond/ZnSe) 3082, 2935, 2838, 1599, 1584, 1515, 1486, 1468, 1428, 1386, 1355, 1331, 1304, 1274, 1256, 1217, 1183, 1148, 1140, 1114, 1090, 1072, 1040, 1018, 1000, 979, 909, 864, 834, 804, 784, 761, 750, 733, 704, 678, 660 cm⁻¹. ¹H NMR (400 MHz, d₆-DMSO) δ 8.45 (d, 1H, J=9.8 Hz, aryl), 8.01 (d, 1H, J=9.8 Hz, aryl), 7.66 (dd, 1H, J=2.2, 8.4 Hz, aryl), 7.53 (d, 1H, J=2.0 Hz, aryl), 7.18-7.25 (m, 3H, aryl), 7.14 (d, 1H, J=8.6 Hz, aryl), 5.02 (m, 1H), 3.75-3.88 (m, 4H), 3.83 (s, 3H, Me), 3.78 (s, 3H, Me), 3.74 (s, 3H, Me), 2.11-2.20 (m, 1H), 1.95-2.01 (m, 1H). ¹³C NMR (100 MHz, d₆-DMSO) δ 152.9, 152.2, 152.0 (2C), 146.7 (2C), 143.5, 126.4, 124.7, 121.1, 119.8, 117.1, 116.9, 115.7, 113.2, 112.9, 112.4, 78.2, 72.2, 66.4, 56.2, 55.7, 55.6, 32.4. LC/MS: RT (min)=4.99; (MH⁺) 449.1. HRMS: (CI+, m/z), calcd for C₂₄H₂₅N₄O₅ (MH⁺), 449.1825; found, 449.1823.

3-(4-methoxyphenyl)-6-(3,4-dimethoxyphenyl)-7H-[1,2,4]-triazolo-[3,4b]-[1,3,4]-thiadiazine (51): ¹H NMR (CDCl₃, 400 MHz) δ 8.09 (dd, 2.4, 6.8 Hz, 2H), 7.57 (d, 2.4 Hz, 1H), 7.42 (dd, 2.0, 8.4 Hz, 1H), 6.99 (dd, 2.0, 6.8 Hz, 2H), 6.96 (d, 8.4 Hz), 3.97 (s, 2H), 3.96 (s, 3H), 3.93 (s, 3H), 3.87 (s, 3H); LC-MS: RT (min)=6.60; [M+H]⁺ 383.1; HRMS calcd for C₁₉H₁₉N₄O₃S (M+H) 383.1100, found 383.1176.

3-(2,3-dimethoxyphenyl)-6-(3,4-dimethoxyphenyl)-7H-[1,2,4]-triazolo-[3,4b]-[1,3,4]-thiadiazine (52): ¹H NMR (CDCl₃, 400 MHz) δ 7.42 (d, 2.0 Hz, 1H), 7.33 (dd, 2.0, 8.4 Hz, 1H), 7.21 (dd, 1.6, 7.6 Hz, 1H), 7.16 (t, 7.6 Hz), 7.08 (dd, 1.6, 7.6 Hz, 1H), 6.89 (d, 8.4 Hz, 1H), 3.97 (s, 2H), 3.92 (s, 3H), 3.89 (s, 3H), 3.84 (s, 3H), 3.75 (s, 3H); LC-MS: RT (min)=6.35; [M+H]⁺413.1; HRMS calcd for C₂₀H₂₁N₄O₄S (M+H) 413.1205, found 413.1281.

3-(2,4-dimethoxyphenyl)-6-(3,4-dimethoxyphenyl)-7H-[1,2,4]-triazolo-[3,4b]-[1,3,4]-thiadiazine (53): ¹H NMR (CDCl₃, 400 MHz) δ 7.59 (d, 8.8 Hz, 1H), 7.43-7.41 (m, 2H), 6.93 (d, 8.8 Hz, 1H), 6.60 (dd, 2.4, 8.8 Hz, 1H), 6.54 (d, 2.4 Hz), 4.06 (s, 2H), 3.93 (s, 3H), 3.87 (s, 3H), 3.86 (s, 3H), 3.75 (s, 3H); LC-MS: RT (min)=6.18; [M+H]⁺ 413.1; HRMS calcd for C₂₀H₂₁N₄O₄S (M+H) 413.1205, found 413.1288.

3-(3,4-dimethoxyphenyl)-6-(3,4-dimethoxyphenyl)-7H-[1,2,4]-triazolo-[3,4b]-[1,3,4]-thiadiazine (54): ¹H NMR (CDCl₃, 400 MHz) δ 7.76-7.73 (m, 2H), 7.56 (d, 2.4 Hz, 1H), 7.44 (dd, 2.0, 8.4 Hz, 1H), 6.98-6.94 (m, 2H), 3.98 (s, 2H), 3.97 (s, 3H), 3.95 (s, 3H), 3.94 (s, 3H), 3.93 (s, 3H); LC-MS: RT (min)=6.22; [M+H]⁺ 413.1; HRMS calcd for C₂₀H₂₁N₄O₄S (M+H) 413.1205, found 413.1279.

3-(3,5-dimethoxyphenyl)-6-(3,4-dimethoxyphenyl)-7H-[1,2,4]-triazolo-[3,4b]-[1,3,4]-thiadiazine (55): ¹H NMR (CDCl₃, 400 MHz) δ 7.65 (d, 1.6 Hz, 1H), 7.41 (d, 2.0 Hz, 1H), 7.39 (d, 2.4 Hz, 2H), 6.95 (d, 8.8 Hz, 1H), 6.58 (t, 2.4 Hz, 1H), 3.98 (s, 2H), 3.97 (s, 3H), 3.95 (s, 3H), 3.83 (s, 6H); LC-MS: RT (min)=6.95; [M+H]⁺ 413.1; HRMS calcd for C₂₀H₂₁N₄O₄S (M+413.1205, found 413.1278.

3-(2-methylphenyl)-6-(3,4-dimethoxyphenyl)-7H-[1,2,4]-triazolo-[3,4b]-[1,3,4]-thiadiazine (56): ¹H NMR (CDCl₃, 400 MHz) δ 7.99 (s, 1H), 7.92 (d, 7.6 Hz, 1H), 7.60 (d, 2.0 Hz, 1H), 7.42-7.35 (m, 2H), 7.30 (d, 7.6 Hz, 1H), 6.96 (d, 8.8 Hz, 1H), 3.98 (s, 2H), 3.97 (s, 3H), 3.93 (s, 3H), 2.42 (s, 3H); LC-MS: RT (min)=7.04; [M+H]⁺ 367.1; HRMS calcd for C₁₉H₁₉N₄O₂S (M+H) 367.1150, found 367.1224.

3-(2-ethoxyphenyl)-6-(3,4-dimethoxyphenyl)-7H-[1,2,4]-triazolo-[3,4b]-[1,3,4]-thiadiazine (57): ¹H NMR (CDCl₃, 400 MHz) δ 7.65 (d, 6.8 Hz, 1H), 7.52 (t, 8.4 Hz, 1H), 7.41 (d, 8.8 Hz, 1H), 7.07 (t, 7.2 Hz, 1H), 6.99 (d, 8.4 Hz, 1H), 6.92 (d, 8.0 Hz, 1H), 4.04 (s, 2H), 3.97 (q, 6.8 Hz, 2H), 3.92 (s, 3H), 3.81 (s, 3H), 1.18 (t, 6.8 Hz, 3H); LC-MS: RT (min)=6.55; [M+H]⁺ 397.1; HRMS calcd for C₂₀H₂₁N₄O₃S (M+H) 397.1256, found 397.1337.

3-(2-hydroxyphenyl)-6-(3,4-dimethoxyphenyl)-7H-[1,2,4]-triazolo-[3,4b]-[1,3,4]-thiadiazine (58): ¹H NMR (CDCl₃, 400 MHz) δ 8.34 (dd, 1.6 Hz, 1H), 7.63 (d, 1.6 Hz, 1H), 7.44 (dd, 2.4, 8.4 Hz, 1H), 7.36 (ddd, 1.6, 7.2, 12 Hz, 1H), 7.13 (dd, 1.2, 8.4 Hz, 1H), 6.92 (ddd, 1.2, 8.4, 15.2 Hz, 1H), 4.00 (s, 2H), 3.98 (s, 3H), 3.97 (s, 3H); LC-MS: RT (min)=7.63; [M+H]⁺ 369.0; HRMS calcd for C₁₈H₁₇N₄O₃S (M+H) 369.0943, found 369.1018.

3-(2-methoxyphenyl)-6-(2,5-dimethoxyphenyl)-7H-[1,2,4]-triazolo-[3,4b]-[1,3,4]-thiadiazine (59): ¹H NMR (CDCl₃, 400 MHz) δ 7.60 (d, 7.6 Hz, 1H), 7.49 (app. t, 8.8 Hz, 1H), 7.07-6.98 (m, 4H), 6.91 (app. d, 10.4 Hz, 1H), 4.00 (s, 2H), 3.85 (s, 3H), 3.77 (s, 3H), 3.72 (s, 3H); LC-MS: RT (min)=6.79; [M+H]⁺ 383.1; HRMS calcd for C₁₉H₁₉N₄O₃S (M+H) 383.1100, found 383.1176.

3-(3-methoxyphenyl)-6-(3-methoxyphenyl)-7H-[1,2,4]-triazolo-[3,4b]-[1,3,4]-thiadiazine (60): ¹H NMR (CDCl₃, 400 MHz) δ 7.85 (d, 8.8 Hz, 2H), 7.67 (d, 7.2 Hz, 2H), 7.36 (t, 8.0 Hz, 1H), 7.01-6.96 (m, 3H), 3.94 (s, 2H), 3.85 (s, 3H), 3.83 (s, 3H); LC-MS: RT (min)=7.21; [M+H]⁺ 353.1; HRMS calcd for C₁₈H₁₇N₄O₂S (M+H) 353.0994, found 353.1068.

3-(4-methoxyphenyl)-6-(4-methoxyphenyl)-7H-[1,2,4]-triazolo-[3,4b]-[1,3,4]-thiadiazine (61): ¹H NMR (CDCl₃, 400 MHz) δ 8.01 (d, 8.8 Hz, 2H), 7.84 (d, 8.4 Hz, 2H), 6.98 (d, 8.4 Hz, 4H), 3.97 (s, 2H), 3.86 (s, 3H), 3.84 (s, 3H); LC-MS: RT (min)=7.02; [M+H]⁺ 353.1; HRMS calcd for C₁₈H₁₇N₄O₂S (M+H) 353.0994, found 353.1075.

3-(2,3-dimethoxyphenyl)-6-(2,5-dimethoxyphenyl)-7H-[1,2,4]-triazolo-[3,4b]-[1,3,4]-thiadiazine (62): ¹H NMR (CDCl₃, 400 MHz) δ 7.20 (dd, 1.6, 7.6 Hz, 1H), 7.14 (t, 8.0 Hz, 1H), 7.10 (d, 3.2 Hz, 1H), 7.06 (dd, 1.6, 8.0 Hz, 1H), 6.99 (dd, 3.2, 9.2 Hz, 1H), 6.88 (d, 9.2 Hz, 1H), 3.98 (s, 2.0), 3.87 (s, 3H), 3.85 (s, 3H), 3.74 (s, 3H), 3.79 (s, 3H); LC-MS: RT (min)=6.93; [M+H]⁺ 413.1; HRMS calcd for C₂₀H₂₁N₄O₄S (M+H) 413.1205, found 413.1288.

3-(2,4-dimethoxyphenyl)-6-(2,5-dimethoxyphenyl)-7H-[1,2,4]-triazolo-[3,4b]-[1,3,4]-thiadiazine (63): ¹H NMR (CDCl₃, 400 MHz) δ 7.52 (d, 8.4 Hz, 1H), 7.04 (d, 3.2 Hz, 1H), 6.98 (dd, 3.2, 9.2 Hz, 1H), 6.90 (d, 8.8 Hz, 1H), 6.57 (2.4, 8.4 Hz, 1H), 6.51 (d, 2.4 Hz, 1H), 3.93 (s, 2H), 3.85 (s, 3H), 3.83 (s, 3H), 3.75 (s, 6H); LC-MS: RT (min)=6.70; [M+H]⁺ 413.0; HRMS calcd for C₂₀H₂₁N₄O₄S (M+H) 413.1205, found 413.1285.

3-(2,5-dimethoxyphenyl)-6-(4-methoxyphenyl)-7H-[1,2,4]-triazolo-[3,4b]-[1,3,4]-thiadiazine (64): ¹H NMR (CDCl₃, 400 MHz) δ 7.74 (d, 8.8 Hz, 2H), 7.16 (d, 2.8 Hz, 1H), 7.01 (dd, 3.2, 9.2 Hz, 1H), 6.90 (dd, 3.6, 9.2 Hz, 3H), 3.94 (s, 2H), 3.81 (s, 3H), 3.76 (s, 3H), 3.63 (s, 3H); LC-MS: RT (min)=6.75; [M+H]⁺ 383.1; HRMS calcd for C₁₉H₁₉N₄O₃S (M+H) 383.1100, found 383.1174.

3-(3,4-dimethoxyphenyl)-6-(2,4-dimethoxyphenyl)-7H-[1,2,4]-triazolo-[3,4b]-[1,3,4]-thiadiazine (65): ¹H NMR (CDCl₃, 400 MHz) δ 7.75 (d, 1.6 Hz, 1H), 7.69 (dd, 1.6, 8.4 Hz, 1H), 7.61 (d, 8.4 Hz, 1H), 6.88 (d, 8.8 Hz, 1H), 6.54 (ddd, 2.0, 8.4, 18 Hz), 3.94 (s, 2H), 3.88 (s, 9H), 3.84 (s, 3H)═; LC-MS: RT (min)=6.78; [M+H]⁺ 413.1; HRMS calcd for C₂₀H₂₁N₄O₄S (M+H) 413.1205, found 413.1208.

3-(3,5-dimethoxyphenyl)-6-(4-methoxyphenyl)-7H-[1,2,4]-triazolo-[3,4b]-[1,3,4]-thiadiazine (66): ¹H NMR (CDCl₃, 400 MHz) δ 7.87 (d, 8.8 Hz, 2H), 7.32 (d, 2.4 Hz, 2H), 6.98 (d, 8.8 Hz, 2H), 6.56 (app. t, 2.4 Hz, 1H), 3.94 (s, 2H), 3.86 (s, 3H), 3.81 (s, 6H); LC-MS: RT (min)=7.36; [M+H]⁺ 383.1; HRMS calcd for C₁₉H₁₉N₄O₃S (M+H) 383.1100, found 383.1181.

3-(2-methylphenyl)-6-(4-methoxyphenyl)-7H-[1,2,4]-triazolo-[1,3,4]-thiadiazine (67): ¹H NMR (CDCl₃, 400 MHz) δ 7.95 (s, 1H), 7.88-7.84 (m, 3H), 7.37 (t, 7.6 Hz, 1H), 7.27 (app. t, 7.6 Hz, 1H), 7.01-6.97 (m, 2H), 3.95 (s, 2H), 3.87 (s, 3H), 2.41 (s, 3H); LC-MS: RT (min)=7.56; [M+H]⁺ 337.1; HRMS calcd for C₁₈H₁₇N₄OS (M+H) 337.1045, found 337.1119.

3-(2-ethoxyphenyl)-6-(4-methoxyphenyl)-7H-[1,2,4]-triazolo-[3,4b]-[1,3,4]-thiadiazine (68): ¹H NMR (CDCl₃, 400 MHz) δ 7.77 (d, 8.8 Hz, 2H), 7.65 (dd, 1.2, 7.6 Hz, 1H), 7.50 (app. t, 7.2 Hz, 1H), 7.09 (t, 7.6 Hz, 1H), 6.96 (dd, 3.6, 8.8 Hz, 3H), 3.96 (q, 6.8 Hz, 2H), 3.95, (s, 2H), 3.85 (s, 3H), 1.12 (t, 6.8, 3H); LC-MS: RT (min)=7.06; [M+H]⁺ 367.1; HRMS calcd for C₁₉H₁₉N₄O₂S (M+H) 367.4368, found 367.1226.

3-(2-hydroxyphenyl)-6-(4-methoxyphenyl)-7H-[1,2,4]-triazolo-[3,4b]-[1,3,4]-thiadiazine (69): ¹H NMR (CDCl₃, 400 MHz) δ 8.33 (d, 8.0 Hz, 1H), 7.91 (d, 8.8 Hz, 2H), 7.35 (t, 7.2 Hz, 1H), 7.12 (d, 8.0 Hz, 1H), 7.05 (d, 8.8 Hz, 2H), 6.95 (t, 7.6 Hz, 1H), 3.97 (s, 2H), 3.90 (s, 3H); LC-MS: RT (min)=8.12; [M+H]⁺ 339.0; HRMS calcd for C₁₇H₁₅N₄O₂S (M+H) 339.0837, found 339.0918.

### Example 2

### Preliminary Results for Inhibitors of PDE4

This Example provides preliminary results illustrating that compounds of the invention with several methoxy substitutions on the adjunct 3- and 6-phenyl rings are good inhibitors of PDE4.

The ability of compounds 71A-K, 72A-K, 73A-K, 74A-K, 75A-K, 76A-K and 77A-K (shown below) to inhibit purified human PDE4A1A (BPS Bioscience, CA) was assessed using IMAP technology (Molecular Devices, CA). Briefly, two microliters of PDE4A1A (0.05 ng/μl PDE4A1A, 10 mM Tris pH 7.2, 0.1% BSA, 10 mM MgCl₂, 1 mM DTT, and 0.05% NaN₃, final concentration) was dispensed into wells of 1536-well black/solid bottom assay plates (Greiner Bio-One North. America, NC) using a Flying Reagent Dispenser (Aurora Discovery, CA). The plates were centrifuged at 1000 rpm for 30 seconds and then 23 nanoliters of test compound was transferred to the assay plate using a Kalypsys pin tool. After incubation at room temperature for 5 min, 2 μL/well of cAMP (100 nM, final concentration) was dispensed for a final assay volume of 4 μL/well. The plates were centrifuged at 1000 rpm for 30 seconds, incubated for 40 minutes at room temperature, and then 4 microliters of IMAP binding reagent were added to the wells. After 1 to 4 hr incubation at room temperature, the fluorescence polarization (FP) signal (Ex=485 nm, Em=530 nm) was measured on Viewlux plate reader (Perkin Elmer, Mass.).

The concentrations at which 50% inhibition of the PDE4 (IC₅₀ values) were observed for these compounds are shown in Table 1 below.

TABLE 1

| 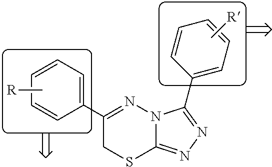 | 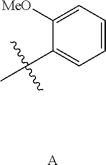 | 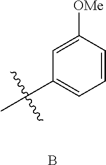 | 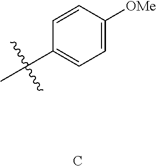 | 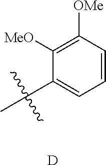 |
|---|---|---|---|---|
| 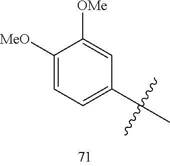 | 0.040* | 0.126 | 0.0889 | 0.112 |
| 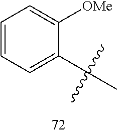 | 20 | 13.4 | 14.1 | >20 |
| 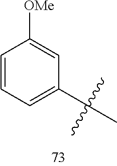 | 12 | 12.6 | 8.4 | >20 |
| 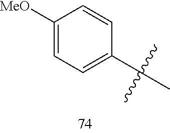 | >20 | 12.6 | 8.4 | >20 |
| 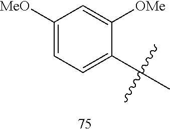 | 15.0 | 14.1 | 13.4 | >20 |
| 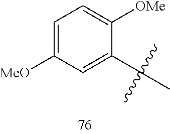 | 8.9 | 12.6 | 10.1 | 11.9 |
| 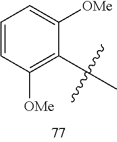 | >20 | >20 | 19.7 | >20 |

| 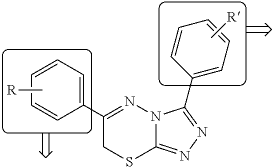 | 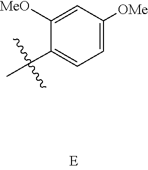 | 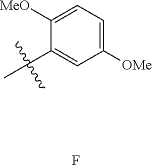 | 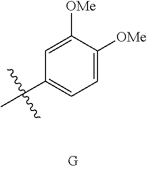 |
|---|---|---|---|
| 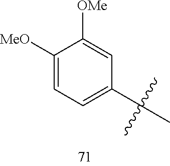 | 0.079 | 0.010 | 0.177 |
| 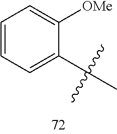 | 19.7 | 19.6 | 8.9 |
| 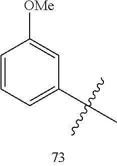 | 13.4 | 8.0 | 9.0 |
| 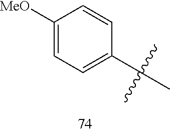 | >20 | 1.7 | 7.5 |
| 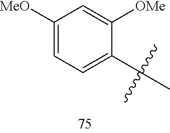 | >20 | 7.5 | 6.7 |
| 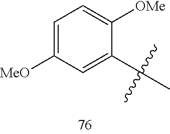 | 5.3 | 11.9 | 6.9 |
| 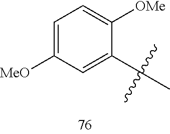 | >20 | >20 | 14.1 |

| 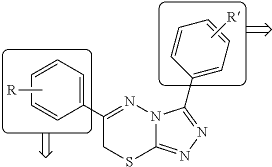 | 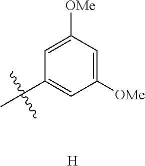 | 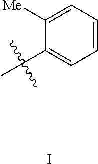 | 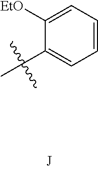 | 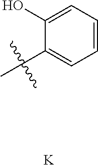 |
|---|---|---|---|---|
| 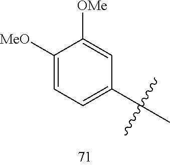 | 0.050 | 0.141 | 0.050 | 0.159 |
| 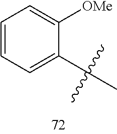 | 13.4 | 14.1 | >20 | >20 |
| 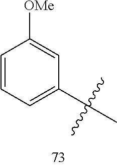 | 13.4 | 3.2 | 11.2 | 14.1 |
| 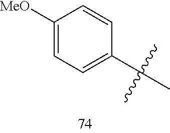 | 9.5 | 12.5 | 4.5 | 8.4 |
| 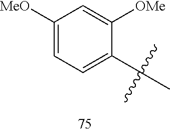 | >20 | >20 | >20 | >20 |
| 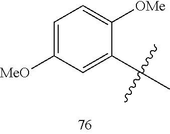 | 12.7 | 6.3 | 13.4 | >20 |
| 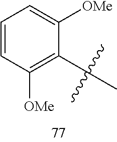 | 19.6 | 14.1 | >20 | >20 |

*IC50 values for the indicated compounds versus PDE4A. SEM values were calculated for compounds 71A-71K and were between +/−0.001 and +/−0.004.

As shown in Table 1, the 3,4-dimethoxy phenyl substitution on the 5 position of the 3,6-dihydro-2H-1,3,4-thiadiazine ring is an important functionality for potent PDE4 inhibition (compounds 71A-71K). All derivatives with this functionality had IC₅₀ values in the low nanomolar range with the most potent being 3-(2,5-dimethoxyphenyl)-6-(3,4-dimethoxyphenyl)-7H-[1,2,4]triazolo[3,4-b][1,3,4]thiadiazine (71F). The phenyl ring attached at the 3 position of the 1,2,4-triazole portion was seemingly less involved in defining the pharmacophore of this structure, as numerous methoxy substitutions had less obvious effects in terms of structure activity relationships.

Crystallographic analyses of several known inhibitors that have such a 3,4-dimethoxy phenyl indicate that a common hydrogen bond between this functionality and a conserved glutamine residue in PDE4 may provide a structural basis for inhibition. Importantly, this glutamine residue (Gln442 in PDE4B and Gln369 in PDE4D) is within close proximity to the coordinated Zn²⁺ and Mg²⁺ ions that form the basis for the mechanism of cAMP hydrolysis by PDE4. Lee et al., FEBS Lett. 2002, 530, 53; Xu et al., Science 2000, 288, 1822.

The strong ability of 3,4-dimethoxy derivatives 71A-71K to inhibit PDE4 indicates that this novel chemotype is inhibiting PDE4 via interaction at the same binding site. Note however that a similar 3,4-dimethoxy substitution pattern engineered upon the phenyl ring attached at the 3 position of the 1,2,4-triazole did not convey favorable affects on PDE4 inhibition as illustrated by compounds 72G, 73G, 74G, 75G, 76G and 77G. These results indicate that the interaction between the 3,4 dimethoxy phenyl moiety attached at the 5 position of the 3,6-dihydro-2H-1,3,4-thiadiazine ring places the remainder of the molecule (i.e. the 1,2,4-triazole and the variously substituted phenyl ring attached at the 3 position) in an orientation that interrupts the binding of cAMP and subsequent hydrolysis.

### Example 3

### Potent Inhibitors of PDE4

This Example illustrates the potency of compounds having the following structures:

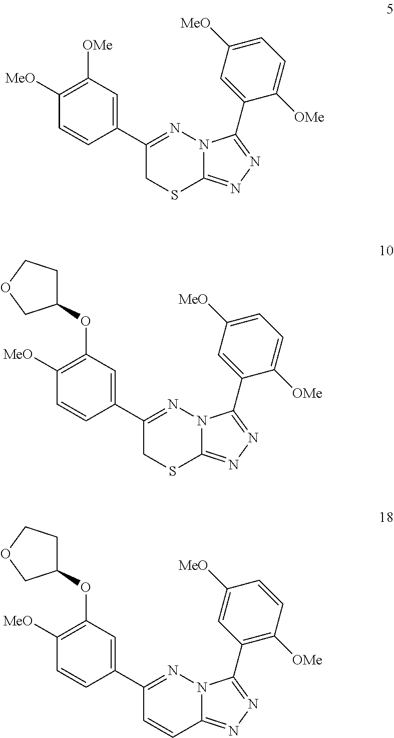

The potency of these compounds was assessed versus 20 different phosphodiesters (PDEs) using methods described in the foregoing Examples. The results of these experiments are summarized in Table 2.

TABLE 2
IC50 or % Inhibition of the Enzyme Activity at 10 μM of the
Compound

| PDE Type | Rolipram | 5 | 10 | 18 |
|---|---|---|---|---|
| PDE1A | NI | NI⁾ | 36% | 32% |
| PDE1B | NI | NI | 52% | 56% |
| PDE1C | NI | 26% | 49% | 74% |
| PDE2A | NI | 41% | 68% | 54% |
| PDE3A | NI | 1.7 μM | 56% | 54% |
| PDE3B | NI | 720 nM | 4.6 μM | 2.3 μM |
| PDE4A1A | 102 nM | 12.9 nM | 0.26 nM | 0.6 nM |
| PDE4B1 | 901 nM | 48.2 nM | 2.3 nM | 4.1 nM |
| PDE4B2 | 534 nM | 37.2 nM | 1.6 nM | 2.9 nM |
| PDE4C1 | 40% | 452 nM | 46 nM | 106 nM |
| PDE4D2 | 403 nM | 49.2 nM | 1.9 nM | 2.1 nM |
| PDE5A1 | NI | 60% | 58% | 51% |
| PDE7A | NI | 73% | 48% | 59% |
| PDE7B | NI | 33% | 43% | 35% |
| PDE8A1 | NI | 57% | 342 nM | 547 nM |
| PDE9A2 | NI | NI | NI | NI |
| PDE10A1 | NI | 823 nM | 632 nM | 388 nM |
| PDE11A4 | NI | NI | NI | NI |

Accordingly, compounds active as phosphodiesterase inhibitors at sub-nanomolar concentrations are provided herein.

### Example 4

### Potent Inhibitors of PDE4

This Example further illustrates that compounds of the invention are excellent inhibitors of PDE4.

PDE4A inhibition profile. The inhibitory potency of compounds of the invention was evaluated against PDE4A using a purified enzyme fluorescence polarization assay (IMAP; Molecular Devices, CA) (see, Skoumbourdis et al. Identification of a potent new chemotype for the selective inhibition of PDE4. Bioorg. Med. Chem. Lett. 2008, 18, 1297-1303).

The results for compounds 7H-[1,2,4]triazolo[3,4-b][1,3,4]thiadiazines 5-10 and the novel [1,2,4]triazolo[4,3-b]pyridazines 17 and 18 are shown in Table 3. For 7H-[1,2,4]triazolo[3,4-b][1,3,4]thiadiazines 5-10, each substitution pattern yielded a molecule with potency in the low nanomolar range.

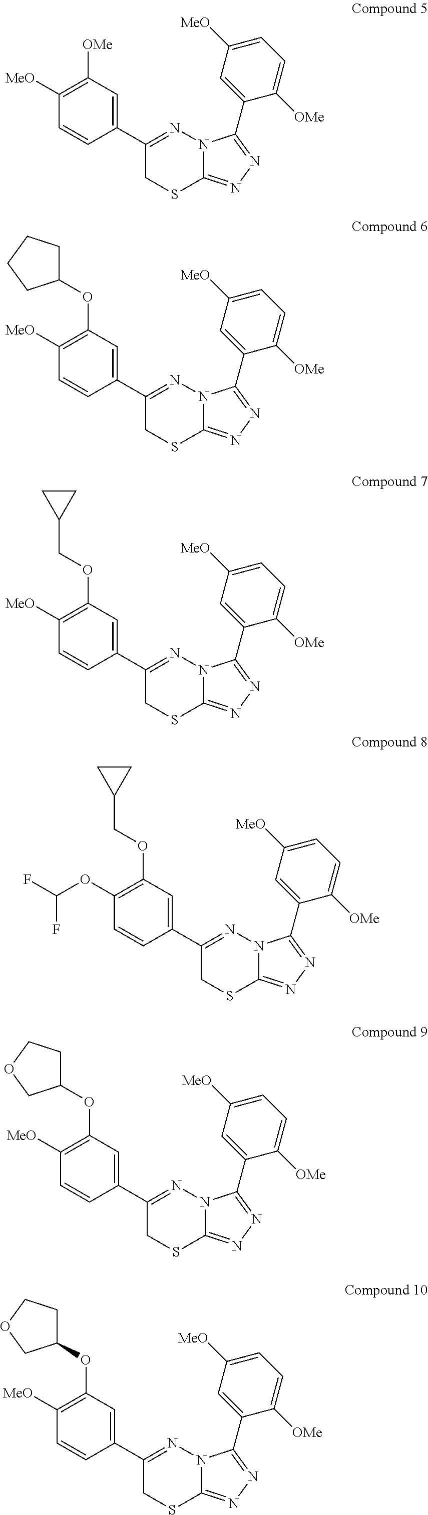

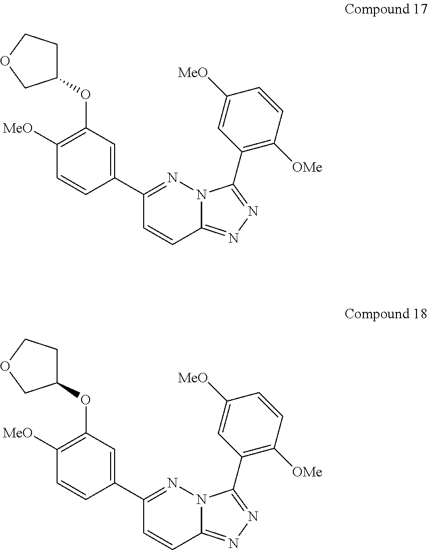

The enantiomerically pure O-(3-THF)[R] substitution of 10 had the best potency with an IC₅₀ value of 3.0 nM. As a result, the enantiomerically pure O-(3-THF)[R] and O-(3-THF)[S] substitutions were incorporated onto the [1,2,4]triazolo[4,3-b]pyridazine core structure and the resulting constructs were found to have excellent potencies for PDE4A inhibition (IC₅₀ value of 7.3±3.8 nM for 17 and 1.5±0.7 nM for 18). Several analogues were also explored with varying substitutions on the phenyl ring attached to the C3 position of the 1,2,4-triazole ring system. Substitutions included methoxy, fluoro, chloro and trifluoromethyl groups on the ortho, meta and para positions of the phenyl ring (see Example 1 for synthetic details and characterization of these analogs). Analysis of these analogs confirmed that various substitutions at one of the ortho positions were important for obtaining potent PDE4A inhibition. Additional substitutions have been shown to be tolerated without effect on the inhibition of PDE4A.

TABLE 3
PDE4A inhibition by compounds 5-10, 17 and 18.
5-10

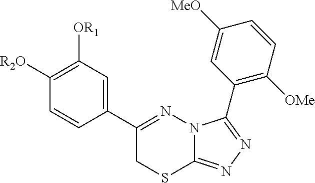

17, 18

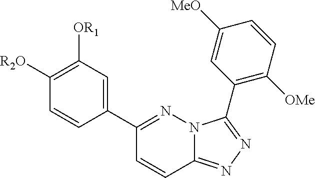

| | | | PDE4A |
|---|---|---|---|
| Analog # | R₁ | R₂ | IC₅₀ (nM) |
| 5 | —CH₃ | —CH₃ | 6.7 ± 0.4 |
| 6 | —Cypent | —CH₃ | 13 ± 0.8 |
| 7 | —CH₂Cyprop | —CH₃ | 6.1 ± 0.9 |
| 8 | —CH₂Cyprop | —CHF₂ | 11 ± 0.7 |
| 9 | -(3-THF)[rac] | —CH₃ | 3.4 ± 0.4 |
| 10 | -(3-THF)[R] | —CH₃ | 3.0 ± 0.2 |
| 17 | -(3-THF)[S] | —CH₃ | 7.3 ± 3.8 |
| 18 | -(3-THF)[R] | —CH₃ | 1.5 ± 0.7 |

*data are from three seperate experiments (SD provided).
Definitions: OCH₃ = methoxy, OCypent = cyclopentyloxy,
OCH₂Cyprop = cyclopropylmethyl, OCHF₂ = 2-difluoromethoxy,
O(3-THF) = O-3-tetrahydrofuranyl [rac = racemic; or R or S
enantiomers].

Selectivity panel of PDE isoforms. Having determined that several compounds had good potency profiles as well as divergent core heterocycles, it was of interest to confirm the selectivity of these agents against a panel of PDE isoforms. To evaluate the activity profile of the synthesized compounds, a panel of 21 phosphodiestrase enzyme isoforms from all eleven primary phosphodiestrase families (except PDE6) was obtained from BPS Bioscience Inc. (11526 Sorrento Valley Rd. Step. A2; San Diego, Calif. 92121). Compounds 5, 10, 18 and 1 were analyzed using this panel of phosphodiesterase enzymes. The resulting IC₅₀ determinations are shown in Table 4.

TABLE 4
PDE isoform selectivity data for 1, 5, 10 and 18.
1

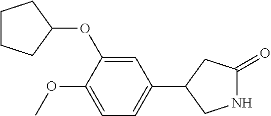

5

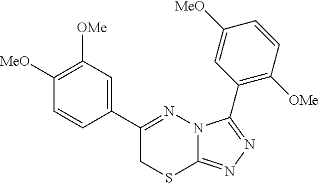

10

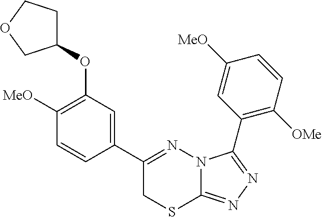

18

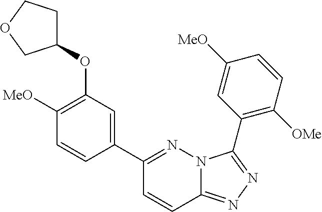

| PDE | 1 | 5 | 10 | 18 |
|---|---|---|---|---|
| isoform* | IC₅₀/% inh. | IC₅₀/% inh. | IC₅₀/% inh. | IC₅₀/% inh. |
| PDE1A | inactive | inactive | 36% | 32% |
| PDE1B | inactive | inactive | 52% | 56% |
| PDE1C | inactive | 26% | 49% | 74% |
| PDE2A | inactive | 41% | 68% | 54% |
| PDE3A | inactive | 1.7 μM | 56% | 54% |
| PDE3B | inactive | 720 nM | 4.6 μM | 2.3 μM |
| PDE4A1A | 102 nM | 12.9 nM | 0.26 nM | 0.6 nM |
| PDE4B1 | 901 nM | 48.2 nM | 2.3 nM | 4.1 nM |
| PDE4B2 | 534 nM | 37.2 nM | 1.6 nM | 2.9 nM |
| PDE4C1 | 40% | 452 nM | 46 nM | 106 nM |
| PDE4D2 | 403 nM | 49.2 nM | 1.9 nM | 2.1 nM |
| PDE5A1 | inactive | 60% | 58% | 51% |
| PDE7A | inactive | 73% | 48% | 59% |
| PDE7B | inactive | 33% | 43% | 35% |
| PDE8A1 | inactive | 57% | inactive | inactive |
| PDE9A2 | inactive | inactive | inactive | inactive |
| PDE10A1 | inactive | 823 nM | 632 nM | 388 nM |
| PDE11A4 | inactive | inactive | inactive | inactive |

*data shown are the IC₅₀ values or the % inhibitions at 10 μm of
compound.

It is apparent from Table 4 that both compounds 10 and 18 are excellent inhibitors of five different isoforms of PDE4. Sub-nanomolar potencies were observed for these compounds against the PDE4A1A enzyme. The modest activities observed against the PDE3B and PDE10A1 enzymes require significantly higher concentrations, indicating that these compounds are sufficiently selective for PDE4 to be useful in vivo as PDE4 inhibitors

Two divergent, cell-based assays of PDE4 activity were used to further evaluate whether the substantial in vitro inhibition of PDE4 by 10 and 18 means that these inhibitors are useful in living cells

Cyclic-nucleotide gated ion channel cell-based assay. The first cell-based analysis of PDE4 activity involved an assay based on the coupling of a constitutively activated G-protein coupled receptor (GPCR) and cyclic-nucleotide gated (CNG) ion channel that are coexpressed in HEK293 cells. See, Titus et al., A Cell-Based PDE4 Assay in 1536-Well Plate Format for High-Throughput Screening. J. Biomol. Screening 2008, 13, 609-618. The read-out for this assay is based on measurement of membrane electrical potential by a potential-sensitive fluorophore (ACTOne™ dye kit). Inhibitors of PDE4 will interfere with the native enzymatic conversion of cAMP to AMP resulting in increased intracellular levels of the cyclic (cAMP) nucleotide due to constitutive activity of the GPCR. In response to increased amounts of cAMP, the CNG ion channel opens resulting in membrane polarization. The dye reacts to this alteration in membrane polarity with an increase in fluorescence detectable by fluorescence spectroscopy of whole cells read on a fluorescence microtiter plate reader.

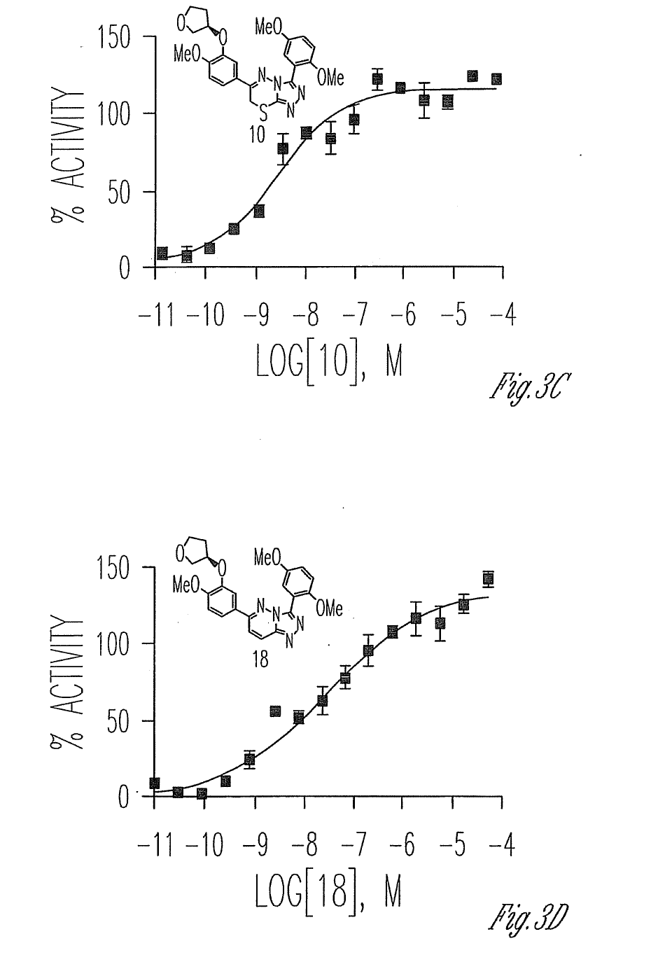

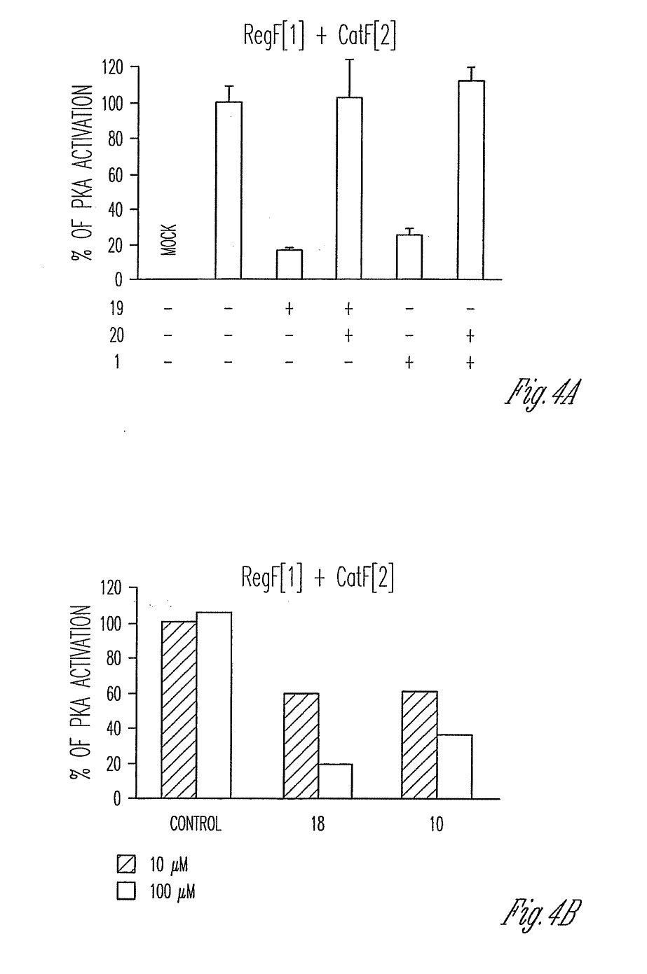

Compounds 5, 10 and 18 were tested using this assay along with the common PDE4 inhibitor 1 (a control). The results are shown in FIG. 3. In this assay, 1 had an effective concentration (EC₅₀) (at 50% activity) of 131.5 nM. In comparison, the triazolothiadiazine based inhibitors were more potent in this cell-based assay, where compounds 5 and 10 had EC₅₀ values of 18.7 and 2.3 nM, respectively. The EC₅₀ of the lone triazolopyridazine 18 was 34.2 nM.

Protein-fragment Complementation (PCA) cell-based assay. PCAs take advantage of the ability of well-engineered protein fragments to form a functional monomer with measurable enzymatic activity when brought into suitable proximity by interacting proteins to which the fragments are fused. Michnick et al., Universal strategies in research and drug discovery based on protein-fragment complementation assays. Nature Rev. Drug Discov. 2007, 6, 569-582; Remy & Michnick, Application of protein-fragment complementation assays in cell biology. BioTechniques 2007, 42, 137-145. To further examine the efficacy of the PDE inhibitors described herein, a reporter enzyme was used—Renilla reniformis luciferase (Rluc), where the N- and C-terminal fragments of Rluc are fused to the catalytic subunits (Cat) and inhibiting regulatory subunits (Reg) of protein kinase A (PKA). Stefan et al., Quantification of dynamic protein complexes using Renilla luciferase fragment complementation applied to protein kinase A activities in vivo. Proc. Natl. Acad. Sci. U.S.A. 2007, 104, 16916-16921. The signaling cascades initiated by GPCR activation are mediated by cAMP production and activation of numerous protein kinases. Negative regulation of these events is solely controlled by the phosphodiesterase class of enzymes. One ubiquitous pathway is activated when cAMP triggers the disassociation of the PKA catalytic and regulatory subunits, which in turn, enables numerous signaling events. In the Rluc PCA PKA reporter, the regulatory subunit II beta cDNA is fused through a sequence coding for a flexible polypeptide linker of ten amino acids (containing eight glycines and two serines) to the N-terminal fragment (Rluc F[1]) [amino acids 1-110 of Rluc] and the cDNA of the PKA catalytic subunit alpha is fused through the same flexible linker to the C-terminal fragment (Rluc F[2]) [amino acids 111-311 of Rluc]. The resulting constructs are designated Reg-F[1] and Cat-F[2] and reconstitute enzymatic activity of Rluc in the absence of cAMP It has been recently demonstrated that this assay could be used to detect the effects of PDE4 inhibition on PKA activation downstream of basal β-2 adrenergic receptor (β₂AR) activities. Stefan et al., Quantification of dynamic protein complexes using Renilla luciferase fragment complementation applied to protein kinase A activities in vivo. Proc. Natl. Acad. Sci. U.S.A. 2007, 104, 16916-16921.

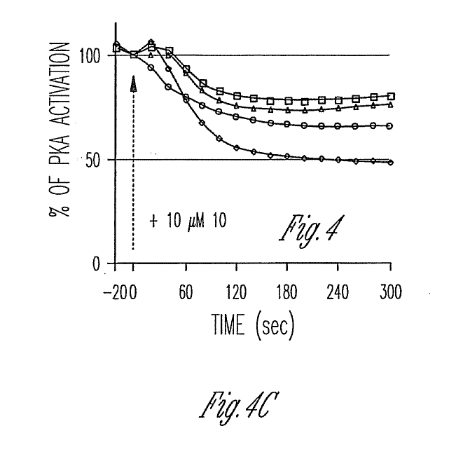

Compounds 1, 10 and 18 were evaluated in this assay using HEK293 cells stably expressing β₂AR and transiently transfected with the required PKA-Rluc fragments [Reg-F[1] and CatF[2]]. The isoproterenol (19) was able to reduce luminescence, indicating dissociation of the Rluc biosensor complex and consequent activation of PKA catalytic activity (FIG. 4A). Pretreatment with the selective β₂AR inverse agonist IC118551 (20), which can decrease basal β₂AR activity, was able to prevent the effects of 19. These controls confirm that alterations of the luminescence signal are primarily mediated through the actions of the β₂AR signaling to PKA. In addition, the effect of 1 confirms the responsiveness of the assay to PDE4 inhibition. Treatment with 10 and 18 at 100 μM and 10 μM concentrations resulted in marked loss of luminescence, indicating that the β₂AR mediated increase of cAMP was due to inhibition of PDE4 (FIG. 4B).

Next, the real-time kinetics of PKA subunit dissociation were examined by administering 10 at a 10 μM concentration. FIG. 4C illustrates these real-time kinetics, which have been normalized to control results observed using 1 μM of the inverse β₂AR agonist 20. In four independent experiments, the presence of 10 reduced the luminescence of the cell-based system by 25% to 50% within 2 minutes of administration (FIG. 4C).

Docking of 10 at PDE4B. Given the potency, selectivity and intracellular inhibition of phosphodiesterase 4, it was of interest to examine the binding of the compounds described herein to the PDE4 structure. The PDE classes of enzymes are comprised of an N-terminal domain, a catalytic domain and a C-terminal domain. Crystallographic analyses of several PDE isozymes have aided researchers in understanding the divergent activities and pharmacology of this class of proteins. Xu et al. Crystal Structures of the Catalytic Domain of Phosphordiesterase 4B Complexed with AMP, 8-Br-AMP and Rolipram. J. Mol. Biol. 2004, 337, 355-365; Xu et al., Atomic Structure of PDE4: Insight into Phosphodiesterase Mechanism and Specificity. Science 2000, 288, 1822-1825. Structures of PDE4 complexed to AMP and several small molecule inhibitors have been reported. Lee et al., FEBS Lett. 2002, 530, 53-58; Huai et al., Biochemistry 2003, 42, 13220-13226; Huai et al., Structure 2003, 11, 865-873; Huai et al., Proc. Nat. Acad. Sci. U.S.A. 2004, 101, 9624-9629; Huai et al. J. Biol. Chem. 2004, 279, 13095-13101.

Such work indicates that the three domains of PDE4 are coordinated through interactions with two metal cations (Zn²⁺ and Mg²⁺). Card et al., Structure 2004, 12, 2233-2247. The residues that coordinate these metals are highly conserved across the PDE family. Both the Zn²⁺ and Mg²⁺ play important roles in the catalytic mechanism of cAMP hydrolysis by coordinating the phosphate moiety. Other important insights include the recognition of a conserved glutamine residue (Q443 in PDE4B) that serves as an important binding residue for the purine motif of cAMP and cGMP.

From numerous crystallographic analyses and modeling efforts it is clear that the catachol diether based inhibitors bind to the catalytic domain of PDE4 through specific hydrogen bonds with the conserved glutamine residue. Initial structure-activity relationship studies of triazolothiadiazine based PDE4 inhibitors indicated that a 3,4-dimethoxy phenyl moiety linked to the C6 position of the 3,6-dihydro-2H-1,3,4-thiadiazine ring was an important substitution pattern for potent PDE4 inhibition. Interestingly, the phenyl ring attached to the C3 position of the 1,2,4-triazole ring system was found to be more amendable to random substitutions without loss of function. This observation suggested that these novel PDE4 inhibitors were binding in a similar orientation to that of compound 1.

Docking simulations were performed to explore this hypothesis using the AutoDock software. Morris et al., J. Comput. Chem. 1998, 19, 1639-1622. The three-dimensional coordinates for PDE4B were retrieved from the Protein Data Bank (PDB ID: 1XMY). Protein and ligand structures were prepared in AutoDock (id.) and the previously reported PDE4-inhibitor complexes were taken into account when preparing the active site grid box. Flexibility was granted to the active site glutamine and the ligand(s). Following multiple docking simulations the most favorable binding conformations were extracted based upon calculated binding constants (reported as Kᵢ values and found to be in the low nanomolar range for favorable docking orientations).

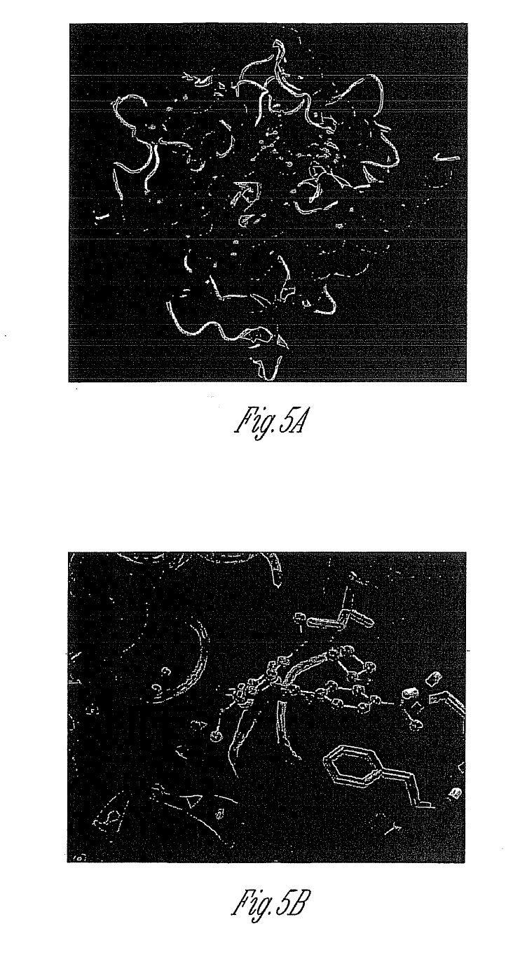

The primary docking modality for compound 10 is shown in FIG. 5. This docking orientation is consistent the formation of an integral hydrogen bond between the catachol diether and Q443 (right panel), while the aromatic moiety is positioned between the conserved isoleucine (I410) and phenylalanine (F446). The remainder of the molecule is shown to extend into the catalytic domain in close proximity to both the Zn²⁺ and Mg²⁺ cations. Such an orientation would block the approach of cAMP to the catalytic domain and forms the basis for inhibiting PDE4. This docking orientation is consistent with the structure-activity relationship observed for the compounds described herein whereby the 3,4-dimethoxy phenyl moiety at the C6 position of the 3,6-dihydro-2H-1,3,4-thiadiazine ring is important for maintaining inhibition in the low nanomolar range whereas the opposite phenyl ring is more amendable to change without significant loss of potency. These observation also indicate that alterations of the core heterocycle from the general triazolothiadiazine structure to the triazolopyridazine structure will have limited affect on the inhibitory profile of these reagents.

While a number of PDE4 inhibitors are currently available, the inventors have discovered a novel class of substituted 6-(3,4-dialkoxyphenyl)-7H-[1,2,4]triazolo[3,4-b][1,3,4]thiadiazines and introduce 6-(3,4-dialkoxyphenyl)-[1,2,4]triazolo[4,3-b]pyridazines as inhibitors of PDE4. As described some of the most potent inhibitors of this important cellular target include compounds with methoxy, cyclopentyloxy, cyclopropylmethoxy, 2-difluoromethoxy and O-3-tetrahydrofuranyl moieties on the left phenyl ring. It was found that the chirally pure R—O-3-tetrahydrofuranyl substitution maintained the best potency in this study.

The compounds of the invention not only possess impressive selectivity and potency for PDE4 versus other PDE family members, but also exhibit excellent activity intracellularly.

PDE4 inhibitors are highly sought after as probes of selected cell signalling pathways and as potential therapeutic agents in diverse areas including memory enhancement and chronic obstructive pulmonary disease (COPD).

All patents and publications referenced or mentioned herein are indicative of the levels of skill of those skilled in the art to which the invention pertains, and each such referenced patent or publication is hereby specifically incorporated by reference to the same extent as if it had been incorporated by reference in its entirety individually or set forth herein in its entirety. Applicants reserve the right to physically incorporate into this specification any and all materials and information from any such cited patents or publications.

The specific methods and compositions described herein are representative of preferred embodiments and are exemplary and not intended as limitations on the scope of the invention. Other objects, aspects, and embodiments will occur to those skilled in the art upon consideration of this specification, and are encompassed within the spirit of the invention as defined by the scope of the claims. It will be readily apparent to one skilled in the art that varying substitutions and modifications may be made to the invention disclosed herein without departing from the scope and spirit of the invention. The invention illustratively described herein suitably may be practiced in the absence of any element or elements, or limitation or limitations, which is not specifically disclosed herein as essential. The methods and processes illustratively described herein suitably may be practiced in differing orders of steps, and that they are not necessarily restricted to the orders of steps indicated herein or in the claims. As used herein and in the appended claims, the singular forms “a,” “an,” and “the” include plural reference unless the context clearly dictates otherwise. Thus, for example, a reference to “an antibody” includes a plurality (for example, a solution of antibodies or a series of antibody preparations) of such antibodies, and so forth. Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the following claims. In addition, where features or aspects of the invention are described in terms of Markush groups, those skilled in the art will recognize that the invention is also thereby described in terms of any individual member or subgroup of members of the Markush group.

1. A compound of formula I: 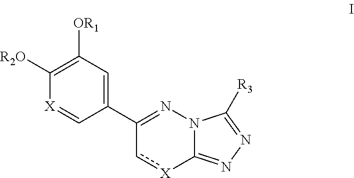
wherein:
X is N or CH in the following ring: 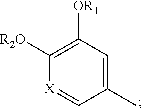
X is CH, CH₂, O, S, N or NH in the following ring: 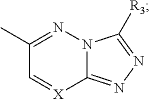
wherein
each R₁ and R₂ is separately alkyl, haloalkyl, cycloalkyl, cycloalkylhalo, heterocycloalkyl, or aryl, where the alkyl, cycloalkyl, cycloalkylhalo, heterocycloalkyl, or aryl can be covalently linked to the oxygen via a lower alkyl; and
R₃ is phenyl substituted with 1-3 substituents independently chosen from alkyl, alkenyl, alkynyl, cycloalkyl, cycloalkenyl, aryl, heteroaryl, OH, O-alkyl, SH, S-alkyl, NH₂, NH-alkyl, N-dialkyl, NH-acyl, NH-aryl, OCO-alkyl, SCO-alkyl, SOH, SO-alkyl, SO₂H, SO₂-alkyl, SO₂NH₂, SO₂NH-alkyl, SO₂N-dialkyl, CF₃, F, Cl, Br, and I groups, such that R₃ comprises an O-alkyl substituent at the 2-position.

2. (canceled)

3. The compound of claim 1, having one of the following formulas: 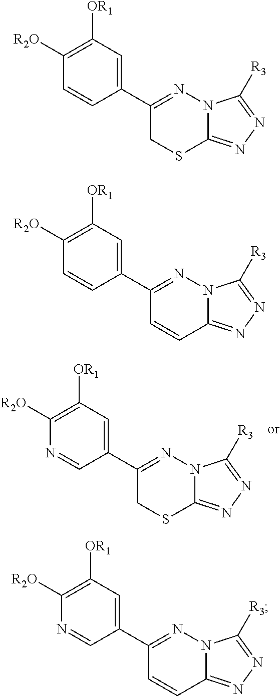
wherein:
each R₁ and R₂ is separately alkyl, haloalkyl, cycloalkyl, cycloalkylhalo, heterocycloalkyl, or aryl, where the alkyl, cycloalkyl, cycloalkylhalo, heterocycloalkyl, or aryl can be covalently linked to the oxygen via a lower alkyl; and
R₃ is phenyl substituted with 1-3 substituents independently chosen from alkyl, alkenyl, alkynyl, cycloalkyl, cycloalkenyl, aryl, heteroaryl, OH, O-alkyl, SH, S-alkyl, NH₂, NH-alkyl, N-dialkyl, NH-acyl, NH-aryl, OCO-alkyl, SCO-alkyl, SOH, SO-alkyl, SO₂H, SO₂-alkyl, SO₂NH₂, SO₂NH-alkyl, SO₂N-dialkyl, CF₃, F, Cl, Br, and I groups, such that R₃ comprises an O-alkyl substituent at the phenyl 2-position.

4. (canceled)

5. The compound of claim 1, wherein X is S or CH in the following ring:

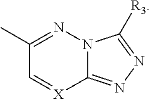

6. The compound of claim 1, wherein R₃ is dimethoxyphenyl.

7. The compound of claim 1, wherein R₃ is:

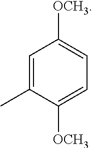

8. The compound of claim 1, wherein the R₁ and R₂ groups are each lower alkyl, cycloalkyl or heterocycloalkyl, where the cycloalkyl, or heterocycloalkyl can be covalently linked to the oxygen via a lower alkyl.

9. The compound of claim 8, wherein R₁ and R₂ lower alkyl groups are each independently methyl or ethyl.

10. The compound of claim 1, wherein the at least one of R₁ and R₂ is a lower alkyl group that is substituted with 1-3 halide atoms.

11. The compound of claim 1, having any of the following formulae: 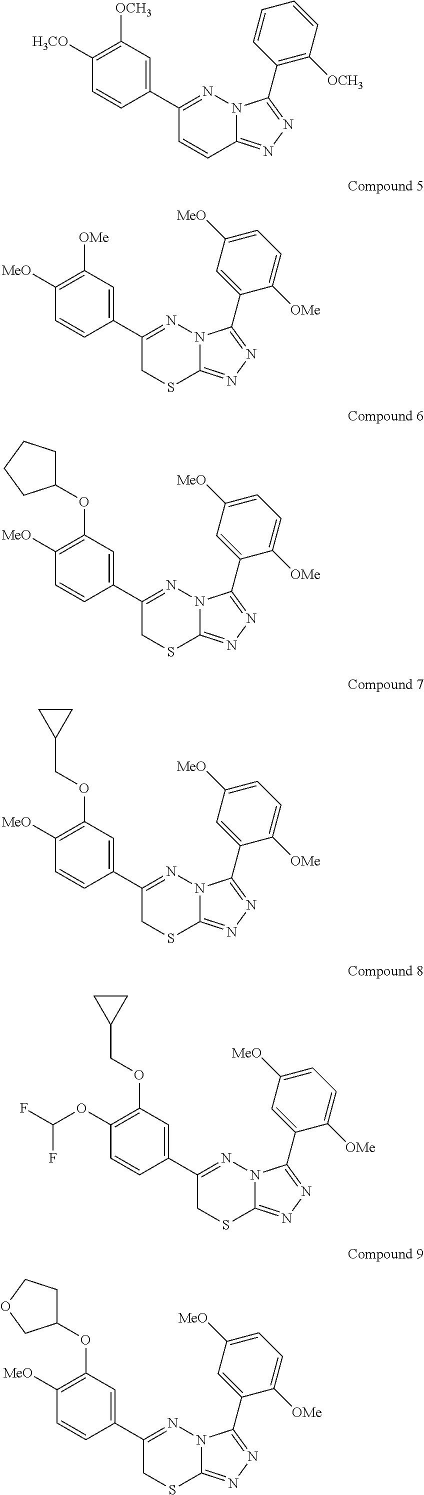 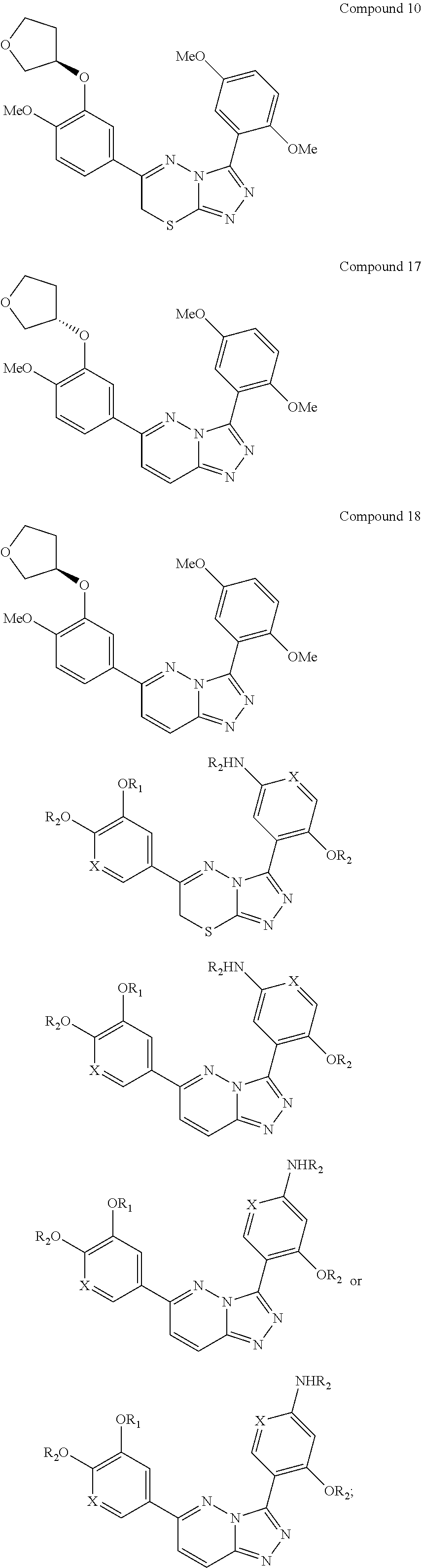
wherein:
X is N or CH in the following ring: 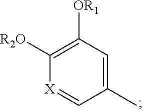
X at other locations is CH; and
each R₁ and R₂ is separately alkyl, haloalkyl, cycloalkyl, cycloalkylhalo, heterocycloalkyl, or aryl, where the alkyl, cycloalkyl, cycloalkylhalo, heterocycloalkyl, or aryl can be covalently linked to the oxygen via a lower alkyl.

12. A composition comprising a carrier and at least one compound of claim 1.

13. The composition of claim 12, wherein the carrier is a pharmaceutically acceptable carrier.

14. The composition of claim 12, wherein the compound of claim 1 is present in the composition in a therapeutically effective amount.

15. A method for inhibiting phosphodiesterase-4 in a mammalian cell, comprising administering to the mammal an effective amount of the composition of claim 13 to thereby inhibit phosphodiesterase-4 in the mammal.

16. The method of claim 15, wherein the effective amount is effective for inhibiting at least 30% of the phosphodiesterase-4.

17. The method of claim 15, wherein the effective amount is effective for inhibiting at least 50% of the phosphodiesterase-4.

18. The method of claim 15, wherein the effective amount is effective for inhibiting at least 60% of the phosphodiesterase-4.

19. (canceled)

20. The method of claim 15, wherein the effective amount of the composition comprises about 0.0001 mg/kg to about 500 mg/kg of a compound of claim 1.

21. The method of claim 19, wherein phosphodiesterase-4 is inhibited within a cell in a mammal to treat any one of the following diseases or disorders: inflammation, acute airway disorders, chronic airway disorders, inflammatory airway disorders, allergen-induced airway disorders, bronchitis, allergic bronchitis, bronchial asthma, emphysema, chronic obstructive pulmonary disease, dermatoses, proliferative dermatoses, inflammatory dermatoses, allergic dennatosis, psoriasis (vulgaris), toxic eczema, allergic contact eczema, atopic eczema, seborrhoeic eczema, Lichen simplex, sunburn, pruritus in the anogenital area, alopecia areata, hypertrophic scars, discoid lupus erythematosus, follicular and widespread pyodermias, endogenous and exogenous acne, acne rosacea, proliferative, inflammatory and allergic skin disorders, rheumatoid arthritis, rheumatoid spondylitis, osteoarthritis, arthritis, AIDS, multiple sclerosis, graft versus host reaction, allograft rejection, shock, septic shock, endotoxin shock, gram-negative sepsis, toxic shock syndrome, adult respiratory distress syndrome, Crohn's disease, ulcerative colitis, inflammatory bowel disease, allergies, allergic rhinitis, sinusitis, chronic rhinitis, chronic sinusitis, allergic conjunctivitis, nasal polyps, cardiac insufficiency, erectile dysfunction, kidney colic, ureter colic in connection with kidney stones, diabetes, diabetes insipidus, cerebral senility, senile dementia (Alzheimer's disease), memory impairment associated with Parkinson's disease or multiinfarct dementia, depression, psychosis, arteriosclerotic dementia or a combination thereof.

22-23. (canceled)

24. A compound of formula I: 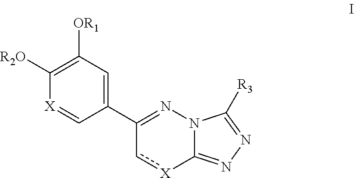
wherein:
X is N or CH in the following ring: 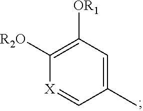
X is CH, CH₂, O, S, N or NH in the following ring: 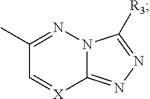
each R₁ cycloalkyl, cycloalkylhalo, heterocycloalkyl, or aryl, where the cycloalkyl, cycloalkylhalo, heterocycloalkyl, or aryl can be covalently linked to the oxygen via a lower alkyl;
each R₂ is alkyl, haloalkyl, cycloalkyl, cycloalkylhalo, heterocycloalkyl, or aryl, where the cycloalkyl, cycloalkylhalo, heterocycloalkyl, or aryl can be covalently linked to the oxygen via a lower alkyl; and
R₃ is aryl substituted with 1-3 substituents independently chosen from alkyl, alkenyl, alkynyl, cycloalkyl, cycloalkenyl, aryl, heteroaryl, OH, O-alkyl, SH, S-alkyl, NH₂, NH-alkyl, N-dialkyl, NH-acyl, NH-aryl, OCO-alkyl, SCO-alkyl, SOH, SO-alkyl, SO₂H, SO₂-alkyl, SO₂NH₂, SO₂NH-alkyl, SO₂N-dialkyl, CF₃, F, Cl, Br, and I groups.

25. A compound according to claim 24, wherein:
R₃ is dimethoxyphenyl; and
X is S or CH in the following ring: [chemical structure]
